(19) United States
(12) Patent Application Publication
(10) Pub. No.: US 2023/0257745 A1
(43) Pub. Date: Aug. 17, 2023

(54) Circular siRNAs

(71) Applicant: ALNYLAM PHARMACEUTICALS, INC., Cambridge, MA (US)

(72) Inventors: Hartmut JAHNS, Cambridge, MA (US); Ivan ZLATEV, Cambridge, MA (US); Muthiah MANOHARAN, Cambridge, MA (US); Christopher S. THEILE, Cambridge, MA (US)

(21) Appl. No.: 18/015,202

(22) PCT Filed: Jul. 9, 2021
(86) PCT No.: PCT/US2021/041015

§ 371 Date: Jan. 9, 2023

Related U.S. Application Data
(60) Provisional application No. 63/050,370, filed on Jul. 10, 2020.

(51) Int. Cl. C12N 15/113

(52) U.S. Cl. CPC C12N 15/113; C12N 2310/14; C12N 2310/321; C12N 2310/322; C12N 2310/315; C12N 2310/351; C12N 2310/532

(57) ABSTRACT

One aspect of the present invention relates to a small circular interfering RNA (sciRNA) comprising a sense strand and an antisense strand, each of said sense and antisense strands comprising at least one nucleic acid modification, optionally wherein the sense strand has a circular or substantially circular structure. Other aspects of the invention relate a pharmaceutical composition and a method for inhibiting the expression of a target gene in a subject using the sciRNA.

This application claims benefit of priority to U.S. Provisional Application No. 63/050,370 filed Jul. 10, 2020, which is herein incorporated by reference in its entirety.

### FIELD OF INVENTION

This invention generally relates to the field of RNA interference with circular-structure siRNAs.

### BACKGROUND

Chemical modifications of the nucleobases, ribose sugar, and phosphate backbone have been used to improve drug-like properties of therapeutic oligonucleotides and to confer favorable pharmacological properties to GalNAc-siRNA conjugates in preclinical and clinical development.

Nevertheless, relatively few alterations have been performed at the level of the three-dimensional structure of siRNAs. Limited examples of supra-RNAi structures have been reported, including hairpin siRNAs (Yu et al., “RNA interference by expression of short-interfering RNAs and hairpin RNAs in mammalian cells,” 99: 6047-52 (2002)), dumbbell-shaped nanocircular siRNAs (Abe et al., “Dumbbell-shaped nanocircular RNAs for RNA interference,” J. Am. Chem. Soc., 129: 15108-09 (2007)), siRNA nanosheets (Kim et al., “Generation of siRNA Nanosheets for Efficient RNA Interference,” Sci. Rep., 6: 25146 (2016)), branched siRNAs (Avino et al., “Branched RNA: A new architecture for RNA interference,” J. Nucleic Acids, 2011: 586935 (2011)), caged circular siRNAs for photomodulation of gene expression (Zhang et al., “Caged circular siRNAs for photomodulation of gene expression in cells and mice,” Chem. Sci., 9: 44-51 (2018)), circular single strand RNAs as siRNA precursors (Kimura et al., “Intracellular build-up RNAi with single-strand circular RNAs as siRNA precursors,” Chem. Commun., 10.1039/C1039CC04872C (2019)), and circular siRNAs with reduced off-target effects (Abe et al., “Synthesis and characterization of small circular double-stranded RNAs,” Chem. Commun., 47: 2125-2127 (2011); Zhang et al., “Circular siRNAs for reducing off-target effects and enhancing long-term gene silencing in cells and mice,” Mol. Ther.-Nucleic Acids, 10: 237-44 (2018)).

However, these reports were based on natural ribonucleotides and phosphodiester linkages, did not utilize therapeutically relevant siRNA chemical modifications, employed inefficient cyclization strategies, such as peptide coupling or T4 ligation techniques, and/or only achieved modest yields. Moreover, some of these reports indicated that cyclizing the antisense (guide) strands into circular siRNAs have resulted in the loss of silencing activity (see e.g., Zhang et al., “Caged circular siRNAs for photomodulation of gene expression in cells and mice,” Chem. Sci., 9: 44-51 (2018)), which may be due to the inability of a circular antisense strand to get loaded onto the Argonaute 2 (Ago2) protein, precluding the formation of an active RNA-induced silencing complex (RISC), the driving component of RNA interference-mediated mRNA silencing. Some of these reports also discouraged chemical modifications, such as modifications with 2′OMe RNA, locked nucleic acid, unlocked nucleoside analogs, 5-nitroindole-modified nucleotide, terminal methylation, backbone phosphothioate, etc., suggesting that these modifications may prevent the loading and processing of sense strand RNA to lower the off-target effect of siRNAs (see e.g., Zhang et al., “Circular siRNAs for reducing off-target effects and enhancing long-term gene silencing in cells and mice,” Mol. Ther.-Nucleic Acids, 10: 237-44 (2018)). In the reports describing an in vivo application, no systemic administration or targeting of exogenous gene expression were described, nor was a therapeutically relevant delivery reagent used for this locally administered high dose siRNA.

Thus, there is a continuing need for a new and improved design for three-dimensional siRNA duplex structure to achieve and enhance the therapeutic potential of RNAi agents, such as enhancing their potency, metabolic stability, and off-target properties.

### SUMMARY

One aspect of the invention relates to a small circular interfering RNA (sciRNA) comprising a sense strand and an antisense strand. Each of the sense and antisense strands comprises at least one nucleic acid modification. To increase the potency, the sciRNA may comprise one or more of (including all of) the following: (a) the antisense strand comprises a phosphate mimic at the 5′-end of an antisense nucleotide sequence, selected from the group consisting of 5′-phosphorothioate (5′-PS), 5′-phosphorodithioate (5′-PS₂), 5′-vinylphosphonate (5′-VP), 5′-methylphosphonate (5′-MePhos), and 5′-deoxy-5′-C-malonyl; (b) all the nucleotides in the sense strand are modified; and (c) all the nucleotides in the antisense strand are modified.

In some embodiments, the sense strand has a circular or substantially circular structure.

In some embodiments, the antisense strand has a circular or substantially circular structure.

In some embodiments, the sense strand is at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. In one embodiment, the sense strand is at least 20 nucleotides in length. In one embodiment, the sense strand is at least 40 nucleotides in length. The sense strand can comprise one or more sense nucleotide sequences.

In some embodiments, the antisense strand is at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. The antisense strand can comprise one or more antisense nucleotide sequences.

The antisense strand is annealed with the sense strand. In some embodiments, one or more sense nucleotide sequences are annealed with the antisense strand. In some embodiments, at least one sense nucleotide sequence is not annealed with the antisense strand.

In some embodiments, the sense nucleotide sequence not annealed with the antisense strand can be a single-stranded oligonucleotide, such as an antisense oligonucleotide (ASO), an antimiR (antagomir) oligonucleotide, or a single-stranded siRNA (ss-siRNA) oligonucleotide.

In some embodiments, a duplex region is formed between the sense strand and antisense strand at least at the seed region of the antisense strand.

In some embodiments, the sense strand comprises at least one sense nucleotide sequence. The at least one sense nucleotide sequence has about 20 to about 45 nucleotides in length. In some embodiments, the antisense strand comprises at least one antisense nucleotide sequence. The at least one antisense nucleotide sequence has about 20 to about 45 nucleotides in length.

In one embodiment, the sense strand is annealed with an antisense strand having about 19 to about 23 nucleotides in length, complementary to a target mRNA transcript nucleotide sequence. In one embodiment, the sense strand is annealed with two or more antisense nucleotide sequences having about 19 to about 23 nucleotides in length, complementary to two or more target mRNA transcript nucleotide sequences.

In some embodiments, the sense strand comprises at least two sense nucleotide sequences. The sense strand comprises at least 40 nucleotides in length. Each of the at least two sense nucleotide sequences has about 18 to about 28 nucleotides in length.

In some embodiments, the antisense strand comprises at least two antisense nucleotide sequences. The antisense strand comprises at least 40 nucleotides in length. Each of the at least two antisense nucleotide sequences has about 18 to about 28 nucleotides in length.

In certain embodiments, the sense strand comprises at least two symmetrical nucleotide sequences, each having about 19 to about 23 nucleotides in length. In one embodiment, the sense strand comprises at least two symmetrical nucleotide sequences, each having 21 nucleotides in length. The sense strand is annealed with an antisense strand comprising two identical antisense nucleotide sequences, each having 23 nucleotides in length and targeting the same mRNA transcript nucleotide sequence.

In certain embodiments, the sense strand comprises at least two asymmetrical nucleotide sequences, each having about 19 to about 23 nucleotides in length. In one embodiment, the sense strand comprises at least two asymmetrical nucleotide sequences, each having 21 nucleotides in length. The sense strand is annealed with an antisense strand comprising two different antisense sequences, each having 23 nucleotides in length and targeting two different mRNA transcript nucleotide sequences.

In some embodiments, the sciRNA further comprises one or more ligands.

In certain embodiments, at least one of the ligands is a lipophilic moiety.

In one embodiment, the lipophilic moiety is lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In one embodiment, the lipophilic moiety contains a saturated or unsaturated C₄-C30 hydrocarbon chain, and an optional functional group selected from the group consisting of hydroxyl, amine, carboxylic acid, sulfonate, phosphate, thiol, azide, and alkyne.

In certain embodiments, at least one of the ligands is a carbohydrate-based ligand. The carbohydrate-based ligand may be D-galactose, multivalent galactose, N-acetyl-D-galactosamine (GalNAc), multivalent GalNAc, D-mannose, multivalent mannose, multivalent lactose, N-acetyl-glucosamine, glucose, multivalent glucose, multivalent fucose, glycosylated polyaminoacids, or lectins.

In certain embodiments, the carbohydrate-based ligand is an ASGPR ligand. For example, the ASGPR ligand is one or more GalNAc derivatives attached through a bivalent or trivalent branched linker, such as:

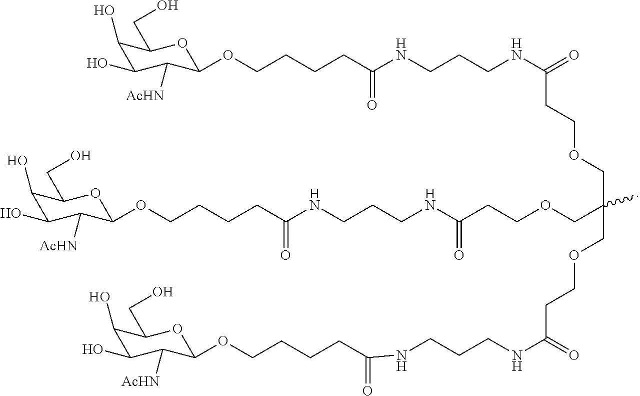 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, at least one of the ligands is conjugated to a strand that has a circular or substantially circular structure. In certain embodiments, at least one of the ligands is conjugated to a strand that does not have a circular or substantially circular structure. In one embodiment, at least one of the ligands is conjugated to a strand that has a circular or substantially circular structure, and at least one of the ligands is conjugated to a strand that does not have a circular or substantially circular structure.

In certain embodiments, at least one of the ligands is conjugated with a sense nucleotide sequence of the sense strand. At least one of the ligands may be conjugated at the 3′-end, 5′-end, or an internal position of the sense nucleotide sequence. In one embodiment, the conjugated sense strand has a circular or substantially circular structure. In one embodiment, the conjugated sense strand does not a circular or substantially circular structure.

In certain embodiments, at least one of the ligands is conjugated with an antisense nucleotide sequence of the antisense strand. At least one of the ligands may be conjugated at the 3′-end, 5′-end, or an internal position of the antisense nucleotide sequence. In one embodiment, the conjugated antisense strand has a circular or substantially circular structure. In one embodiment, the conjugated antisense strand does not a circular or substantially circular structure.

In some embodiments, the sciRNA further comprises at least one chemical modification. The chemical modification may include an internucleoside linkage modification, a nucleobase modification, a sugar modification, or combinations thereof.

In certain embodiments, the chemical modification is selected from the group consisting of LNA, ENA, HNA, CeNA, 2′-O-methoxyalkyl (e.g., 2′-O-methoxymethyl, 2′-O-methoxyethyl, or 2′-O-2-methoxypropanyl), 2′-O-alkyl, 2′-O-allyl, 2′-C-allyl, 2′-fluoro, 2′-deoxy, 2′-O—N-methylacetamido (2′-O-NMA), 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl, and combinations thereof.

In certain embodiments, the chemical modification is a 2′-modification selected from the group consisting of 2′-O-methyl, 2′-deoxy, 2′-fluoro, and combinations thereof.

In some embodiments, about 100%, 95%, 90%, 85%, 80%, 75%, 70%, 65%, 60%, 55%, 50%, 45%, 40%, 35% or 30% of all the nucleotides are modified. For example, when 50% of all the nucleotides are modified, 50% of all nucleotides present in the sciRNA contain a modification as described herein.

In some embodiments, all the nucleotides in the sense strand are modified. In one embodiment, each of the nucleotides in the sense strand is independently modified with a 2′-modification selected from the group consisting of 2′-O-methyl, 2′-deoxy, 2′-fluoro, and combinations thereof,

In some embodiments, all the nucleotides in the antisense strand are modified. In one embodiment, each of the nucleotides in the antisense strand is independently modified with a 2′-modification selected from the group consisting of 2′-O-methyl, 2′-deoxy, 2′-fluoro, and combinations thereof,

In one embodiment, at least 50% of the nucleotides of the sciRNA are independently modified with 2′-O-methyl, 2′-O-allyl, 2′-deoxy, or 2′-fluoro.

In some embodiments, the sense strand contains a nucleotide-based linker (tether). In some embodiments, the sense strand contains a non-nucleotide-based linker (tether). In one embodiment, the sense strand comprises more than one sense nucleotide sequences, and the sense nucleotide sequences are connected to each other via a nucleotide-based or a non-nucleotide-based linker (tether).

In some embodiments, the antisense strand contains a nucleotide-based linker (tether). In some embodiments, the antisense strand contains a non-nucleotide-based linker (tether). In one embodiment, the antisense strand comprises more than one antisense nucleotide sequences, and the antisense nucleotide sequences are connected to each other via a nucleotide-based or a non-nucleotide-based linker (tether).

In certain embodiments, the nucleotide-based or non-nucleotide-based linker (tether) is a stable linker (tether) that is stable in a biological fluid. For instance, the nucleotide-based or non-nucleotide based stable linker (tether) is stable in plasma or artificial cerebrospinal fluid.

In certain embodiments, the nucleotide-based or non-nucleotide-based linker (tether) is a cleavable linker (tether). For instance, the nucleotide-based or non-nucleotide based cleavable linker (tether) can be cleavable in liver homogenates, liver tritosomes, liver lysosomes, liver cytosol, brain homogenates, brain tritosomes, brain lysosomes, or brain cytosol.

In certain embodiments, the cleavable linker (tether) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., an ester group), or a peptidase cleavable linker (e.g., an ester group).

In other embodiments, at least one of the cleavable linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the cleavable linker comprises at least one modified internucleotide linkage selected from the group consisting of a phosphodiester, phosphotriester, hydrogen phosphonate, alkyl or aryl phosphonate, phosphoramidate, phosphorothioate, methylenemethylimino, thiodiester, thionocarbamate, N,N′-dimethylhydrazine, phosphoroselenate, borano phosphate, borano phosphate ester, amide, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, oxime, methyleneimino, methylenecarbonylamino, methylenehydrazo, methylenedimethylhydrazo, methyleneoxymethylimino, ether, thioacetamido, and combinations thereof.

In some embodiments, the sense strand contains a nucleotide-based cleavable linker (tether) that is cleavable by DICER. In some embodiments, the sense strand comprises a substrate cleavable by DICER.

In some embodiments, the antisense strand contains a nucleotide-based cleavable linker that is cleavable by DICER. In some embodiments, the antisense strand comprises a substrate cleavable by DICER.

In some embodiments, the ligand may be conjugated to the sciRNA via a direct attachment to the ribosugar of the sciRNA. Alternatively, the ligand may be conjugated to the sciRNA via one or more linkers (tethers), and/or a carrier.

In some embodiments, the ligand may be conjugated to the sciRNA via a carrier that replaces one or more nucleotide(s). The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

In some embodiments, the sense strand forms circular or substantially circular structure via a cycling linking moiety that connects one end of the sense strand to the other end of the sense strand.

In some embodiments, the antisense strand forms circular or substantially circular structure via a cycling linking moiety that connects one end of the antisense strand to the other end of the antisense strand.

In certain embodiments, the cycling linking moiety may contain one or more linkages selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In certain embodiments, the cycling linking moiety may contain one or more cyclic groups selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl.

In certain embodiments, the cycling linking moiety also serves as the carrier that carries a ligand and connect the ligand to the sciRNA.

In some embodiments, the antisense strand comprises a phosphate or phosphate mimic at the 5′-end of a strand, either at the sense strand or antisense strand or both. In one embodiment, the phosphate mimic is at the 5′ end of an antisense nucleotide sequence.

The phosphate mimic can be 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

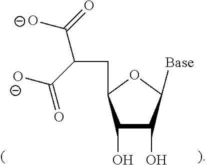

In one embodiment, the phosphate mimic is a 5′-vinylphosphonate (VP). The 5′-VP can be either 5′-E-VP isomer (i.e., trans-vinylphosphate,

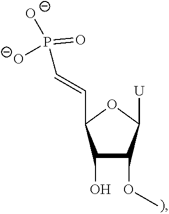

5′-Z-VP isomer (i.e., cis-vinylphosphate,

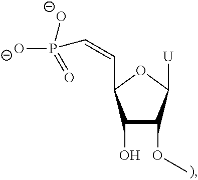

or mixtures thereof.

In one embodiment, the phosphate mimic is a 5′-vinyl phosphonate (VP).

In certain embodiments, the antisense strand forms circular or substantially circular structure, and contains a cleavable linker (nucleotide or non-nucleotide) capable of generating a metabolite of a 5′-monophosphate at the antisense strand. The circular or substantially circular antisense strand can be cleaved to a linear structure that contains a metabolite of a 5′-monophosphate at the antisense strand.

In some embodiments, the sciRNA further comprises at least one terminal, chiral phosphorus atom.

A site specific, chiral modification to the internucleotide linkage may occur at the 5′ end, 3′ end, or both the 5′ end and 3′ end of a sense or antisense nucleotide sequence. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a sense or antisense nucleotide sequence. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in WO 2019/126651A1, which is incorporated herein by reference in its entirety.

In some embodiments, the sciRNA comprises at least two blocks of two consecutive phosphorothioate or methylphosphonate internucleotide linkage modifications.

In some embodiments, the sciRNA has at least two phosphorothioate internucleotide linkages at the first five nucleotides on the antisense strand (counting from the 5′ end).

In some embodiments, an antisense nucleotide sequence of the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In one embodiment, the antisense strand comprises at least two consecutive phosphorothioate internucleotide linkage modifications within positions 18-23 of an antisense nucleotide sequence, counting from the 5′-end of the antisense nucleotide sequence. The sense strand comprises at least two consecutive phosphorothioate internucleotide linkage modifications within position 1-5 of the sense nucleotide sequence, counting from the 5′-end of the sense nucleotide sequence.

In some embodiments, the sciRNA comprises the following features:

all the nucleotides in the sense strand and antisense strand are modified;

the sciRNA comprises at least two blocks of two consecutive phosphorothioate or methylphosphonate internucleotide linkage modifications, and the sciRNA comprises one or more ligands.

In one embodiment, the sciRNA comprises the following features:

all the nucleotides in the sense strand and antisense strand are modified with a 2′-O-methyl or 2′-fluoro modification;

the antisense strand comprises at least two consecutive phosphorothioate internucleotide linkage modifications within positions 18-23 of an antisense nucleotide sequence, counting from the 5′-end of the antisense nucleotide sequence; and the sense strand comprises at least two consecutive phosphorothioate internucleotide linkage modifications within position 1-5 of the sense nucleotide sequence, counting from the 5′-end of the sense nucleotide sequence; and

the sciRNA comprises at least one carbohydrate-based ligand conjugated with a sense nucleotide sequence of the sense strand, at the 3′-end of the sense nucleotide sequence.

Another aspect of the invention relates to a pharmaceutical composition comprising a sciRNA and a pharmaceutically acceptable excipient. The sciRNA comprises a sense strand and an antisense strand. Each of the sense and antisense strands comprises at least one nucleic acid modification. All the above embodiments relating to the sense strand, antisense strand, the chemical modifications on the sense and antisense strand, the nucleotide-based and non-nucleotide-based linkers, the ligand conjugation, and the cycling linking moiety disclosed in the first aspect of the invention relating to the sciRNA are suitable in this aspect of the invention relating to a pharmaceutical composition.

Another aspect of the invention relates to a method for inhibiting the expression of a target gene in a subject, comprising administering to the subject a sciRNA, in an amount sufficient to inhibit expression of the target gene. The sciRNA comprises a sense strand and an antisense strand. Each of the sense and antisense strands comprises at least one nucleic acid modification. All the above embodiments relating to the sense strand, antisense strand, the chemical modifications on the sense and antisense strand, the nucleotide-based and non-nucleotide-based linkers, the ligand conjugation, and the cycling linking moiety disclosed in the first aspect of the invention relating to the sciRNA are suitable in this aspect of the invention relating to a method for inhibiting the expression of a target gene in a subject.

### BRIEF DESCRIPTION OF THE DRAWINGS

FIG. 1A is a schematic representation of an exemplary GalNAc-sciRNA duplex. FIG. 1B illustrates the chemical modifications used in the exemplary GalNAc-sciRNA duplex.

FIGS. 2A-2D are graphs of decay curves of enzymatic digestion using single-stranded poly 2′-deoxy linear and circular oligonucleotides (ON-3 and ON-4, respectively) and single-stranded fully 2′-modified linear and circular oligonucleotides (ON-5 and ON-6, respectively) in an in vitro assay using either a 3′-exonuclease (FIG. 2A and FIG. 2B) or 5′-exonuclease (FIG. 2C and FIG. 2D).

FIGS. 3A-3B are graphs showing the stability of full-length sense strand after incubation of the duplex in plasma and liver homogenate measured using LC-MS. FIG. 3A shows the mean natural logarithm of the percent of sense strand remaining in rat plasma. FIG. 3B shows the mean natural logarithm of the percent of sense strand remaining in rat liver homogenate. Plotted are means. Error bars are standard deviation of three replicates per time point.

FIG. 4 is a graph showing imino regions of 1D ¹H NMR spectra of linear structure GalNAc-siRNAs (Table 4, si-1, si-2, si-3 and si-6) and cyclic structure GalNAc-sciRNA duplexes (Table 4, si-4 and si-5). The imino protons engaged in Watson-Crick base pairs display chemical shift values in the range from δ 12 to 14 ppm.

FIGS. 5A-5B are graphs of pharmacodynamics profiles after a single subcutaneous administration of linear GalNAc-siRNA (Table 4, si-1, si-2 and si-6) and circular GalNAc-sciRNA (Table 4, si-4, si-5 and si-7) conjugates in mice. A single dose of each conjugate (3 mg/kg) was administered in mice on Day 0, and serum was collected on Days 0 (pre-dose), 3, 7 and 14. Circulating serum protein levels for TTR (FIG. 5A) and C5 (FIG. 5B) were determined using an appropriate ELISA kit, relative to PBS groups. Error bars are SD (n=3).

FIGS. 6A-6B are graphs showing the whole liver and Ago2 levels of antisense strand for linear GalNAc-siRNA (Table 4, si-1, si-2 and si-3) and circular GalNAc-sciRNA (Table 4, si-4 and si-5) conjugates in mice. FIG. 6A shows the liver levels of antisense strands isolated and measured from whole mouse livers. FIG. 6B shows the levels of antisense strand isolated and measured from immunoprecipitated Ago2 from whole mouse livers. A single dose of each conjugate (3 mg/kg) was administered in mice on Day 0, and livers were collected on Day 7. Levels were determined using SL-RT QPCR relative to PBS groups. Error bars are SD (n=3).

FIG. 7 illustrates a model of a sciRNA:Ago2 complex based on the crystal structure of Ago2 bound to duplex RNA with seed region pairing. Z linker carbons are highlighted. Selected side chains of the Ago2 PIWI and MID domains and L2 linker region are labeled. The view is across the major (top) and minor grooves (bottom) of the seed region duplex.

### DETAILED DESCRIPTION

The inventors have designed a novel strategy to prepare a small circular interfering RNAs (sciRNAs) using chemically modified nucleotides and connecting the extremities of the nucleic acids of a sense strand, generating a circular sense construct with blocked 5′ and 3′ ends. For instance, exemplary sciRNAs have been synthesized with the antisense strand annealed to a 5′-3′ cyclized sense strand carrying a trivalent GalNAc ligand, prepared using “click” chemistry, and potent gene expression silencing in vitro and in vivo have been observed with these sciRNAs, especially the ones with all the nucleotides in the sense strand modified, all the nucleotides in the antisense strand modified, or phosphate mimic modifications at the 5′-end of an antisense nucleotide sequence, including, for instance, 5′-phosphorothioate (5′-PS), 5′-phosphorodithioate (5′-PS₂), 5′-vinylphosphonate (5′-VP), 5′-methylphosphonate (5′-MePhos), and 5′-deoxy-5′-C-malonyl modifications.

One aspect of the invention relates to a small circular interfering RNA (sciRNA) comprising a sense strand and an antisense strand. Each of the sense and antisense strands comprises at least one nucleic acid modification.

Circular sciRNAs Structure Design

In some embodiments, the sense strand has a circular or substantially circular structure.

In some embodiments, the antisense strand has a circular or substantially circular structure.

The sense strand or antisense strand can form circular or substantially circular structure via a cycling linking moiety that connects one end of the sense (or antisense) strand to the other end of the sense (or antisense) strand. The circular or substantially circular structure of the sense or antisense strand may be formed by a cyclization procedure illustrated in Scheme 1. As shown in Scheme 1, a reactive linking moiety Q is added to one end of the sense (or antisense) strand and another reactive linking moiety Y is added to the other end of the sense (or antisense) strand. Q and Y each may contain various linkers (tethers) and carrier(s) which may carry ligand(s), and each contain a terminal functional group that are reactive to each other. Activating the reaction between Q and Y via an addition reaction would then form Z, a cycling linking moiety, which closes the cycle, forming a circular or substantially circular structure. An exemplary cyclization procedure via a click chemistry (e.g., forming a triazole from the azide-alkyne cycloaddition) is illustrated in Scheme I of Example 1.

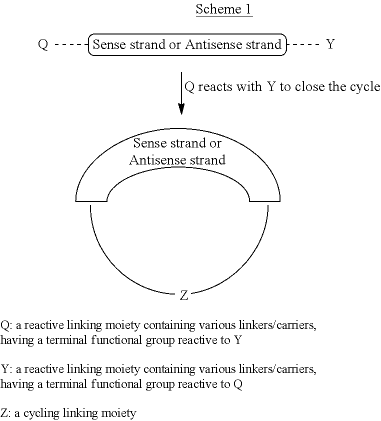

Depending on the reactions used for the cyclization of the sense (or antisense) strand, and the linkers/cyclic groups contained in the reactive linking moieties Q and Y, the cycling linking moiety Z in the circular sense (or antisense) strand may contain one or more linkages selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In one embodiment, the cycling linking moiety Z in the circular sense (or antisense) strand may contain one or more linkages selected from the group consisting of a triazole linkage, an amide linkage, a disulfide linkage, a phosphate linkage, an oxime linkage, an alkyl linkage, a PEG linkage, an ether linkage, a thioether linkage, an urea linkage, a carbonate linkage, an amine linkage, a maleimide-thioether linkage, a phosphodiester linkage, a sulfonamide linkage, a carbamate linkage, and combinations thereof.

In certain embodiments, the cycling linking moiety may further contain one or more carriers that may serve to connect a ligand to the sciRNA. The carrier may be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

One exemplary cycling linking moiety contains a triazole linkage formed through a cyclization procedure via a click chemistry (e.g., from the azide-alkyne cycloaddition). As discussed above in Scheme 1, the cyclization can be formed by attaching a reactive linking moiety Q to one end of the sense (or antisense) strand, attaching another reactive linking moiety Y to the other end of the sense (or antisense) strand, and activating the reaction between Q and Y. The Q/Y pair in this case is azide/alkyne pair. Non-limiting exemplary molecules that contain the reactive linking moiety Q/Y (in this case, azide/alkyne functional groups) are illustrated below.

| | |
|---|---|
| L123 N-(hexynylcarb- oxamidocaproyl)-4- hydroxyprolinol (Hyp- hexynyl) | 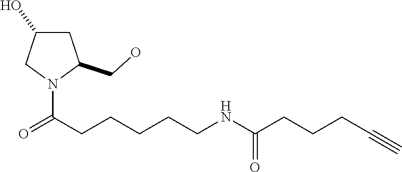 [SMILES: C#CCCCC(=O)NCCCCCC(=O)N1C[C@H](O)C[C@H]1CO] |
| L146 N-(benzocyclooctynyl- carboxamidocaproyl- amidocaproyl)- 4-hydroxyprolinol (Hyp-C6-C6- octynyl) | 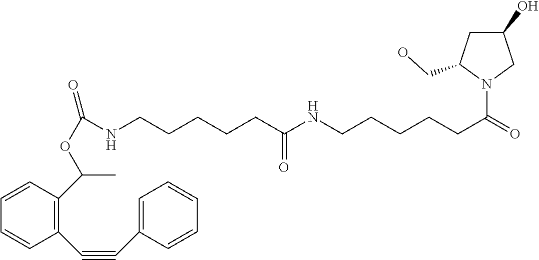 [SMILES: CC(OC(=O)NCCCCCC(=O)NCCCCCC(=O)N1C[C@H](O)C[C@H]1CO)c1ccccc1C#Cc1ccccc1] |
| L177 N-(azido-PEG4- carboxamidocaproyl)-4- hydroxyprolinol (Hyp- C6-PEG4- N3) | 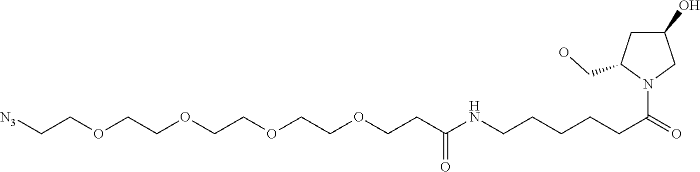 [SMILES: [N-]=[N+]=NCCOCCOCCOCCOCCC(=O)NCCCCCC(=O)N1C[C@H](O)C[C@H]1CO] |
| L347 N-(propargyl ether carboxamidocaproyl)-4- hydroxyprolinol | 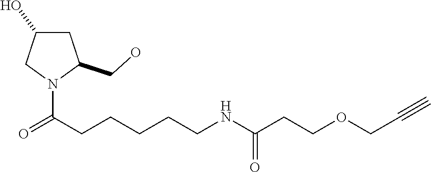 [SMILES: C#CCOCCC(=O)NCCCCCC(=O)N1C[C@H](O)C[C@H]1CO] |
| Q44 N-(hexinylcarb- oxamidocaproyl)- prolinol-4-phosphate (Hyp-hexinyl) | 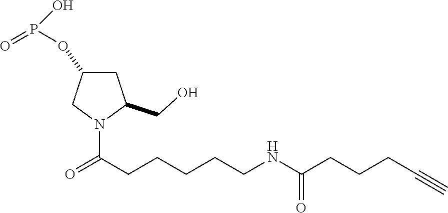 |
| Q84 N-(hexynyl)-prolinol-4- phosphate (Hyp-hexynyl) L124 N-(hexynyl)-4- hydroxyprolinol | 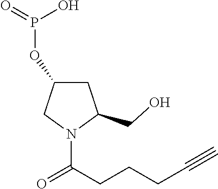 |
| Q99 N-(benzocyclooctynyl- carboxamidocaproyl- amidocaproyl)- prolinol-4-phosphate (Hyp-C6-C6- octynyl) | 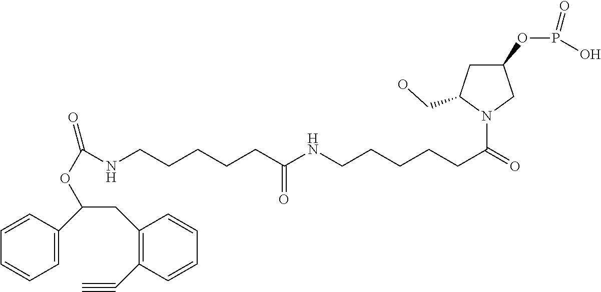 |
| Q127 N-(azido-PEG4- carboxamidocaproyl)- prolinol-4- phosphate (Hyp-C6- PEG4-N3) | 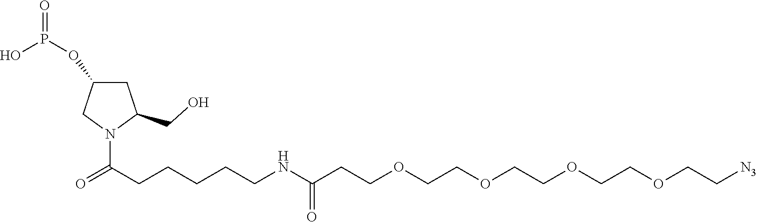 |
| Q136 N-(6- Azidohexylcarb- oxamidocaproyl)- prolinol-4-phosphate (Hyp-C6-C6- Azido) | 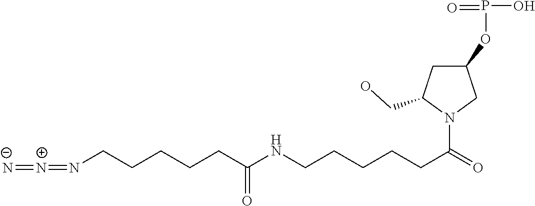 |
| Q325 Q8 with C6-DBCO | 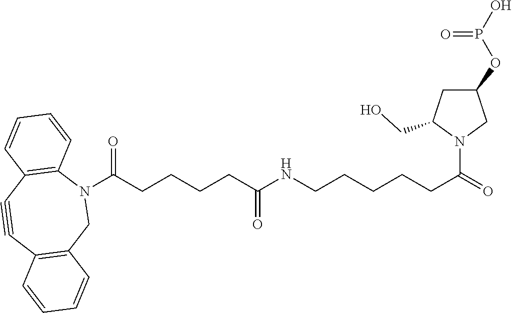 |
| Q186 5-hexyn-1yl-phosphate | 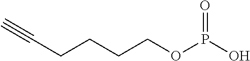 |
| Q187 10-(6-oxo-6- (dibenzo[b,f]azacyclooct- 4-yn-1- yl)-capramido-N-ethyl)-O- triethyleneglycol-1- phosphate (DBCO-TEG-(10-1941)) | 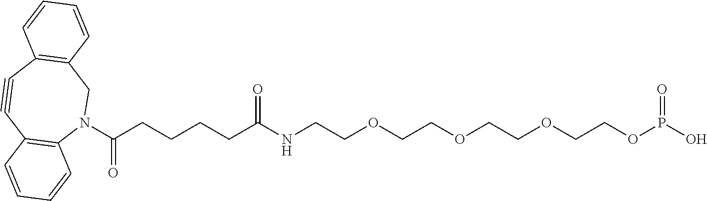 |
| Q301 6-azidohexyl phosphate | 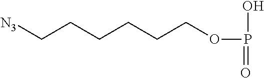 |
| Q301S 6-azidohexyl phosphorothioate | 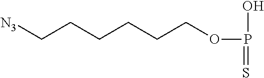 |
| Q396 N-(aminocaproyl-propargyl ether)prolinol-4-phosphate | 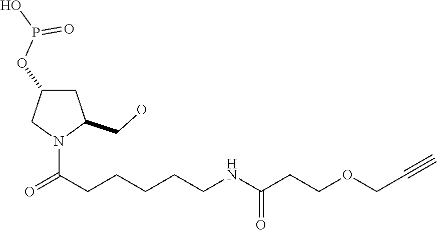 |
| Q397 N-(aminocaproyl- propargyl ether)- (S)-pyrrolidin-3- ol-phosphate | 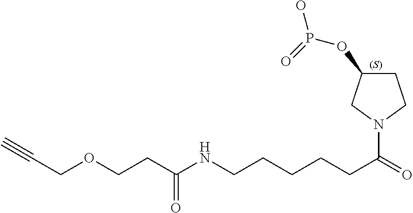 |
| Q399 2,3,5,6-tetrafluoro-4-azido- benzoyl-N- aminocaproyl-(S)- pyrrolidin-3-ol-phosphate | 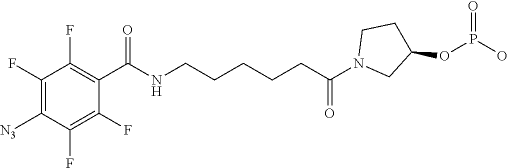 |
| Q422 N-(hexynylcarb- oxamidocaproyl)- (S)-pyrrolidin-3-ol- phosphate | 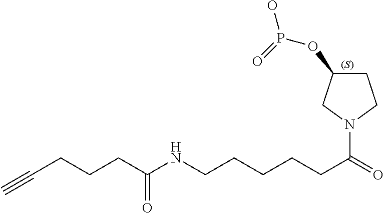 |
| Apy 2′-O-propynyl-adenosine-3′- phosphate | 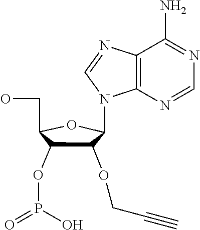 |
| Cpy 2′-O-propynyl-cytidine-3′- phosphate | 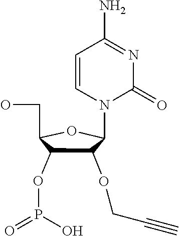 |
| Upy 2′-O-propynyl-uridine-3′- phosphate | 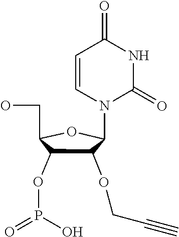 |
| Tpy 2′-O-propynyl-5- methyl-uridine- 3′-phosphate | 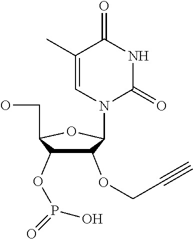 |
| Gpy 2′-O-propynyl- guanosine-3′- phosphate | 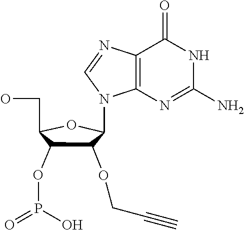 |

As discussed above, these exemplary molecules may be attached to the end of an oligonucleotide strand via, e.g., a phosphate. Activating the click chemistry between the reactive linking moieties between the Q/Y pair would form a cyclized oligonucleotide strand. For instance, attaching L123 and Q301 (illustrated in the above table) to each end of an oligonucleotide strand via a phosphate and clicking the azide/alkyne pair in L123 and Q301 would form

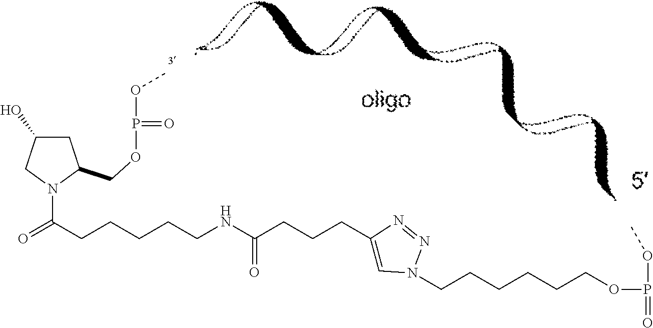

(Z49—cyclization by clicking 3′-phosphate-Hyp-C9-1,4-triazole-C6-5′-phosphate).

Additional non-limiting examples of the cycling linking moieties Z formed by clicking the above-illustrative reactive linking moiety Q/Y pairs are illustrated below.

| | | |
|---|---|---|
| Q310 | Click Q187 and Q127 | 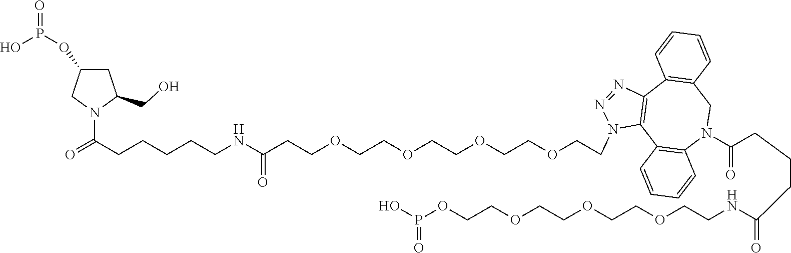 |
| Q324 (DBCO- azide 5′-3′ link) | Click Q187 and L177 | 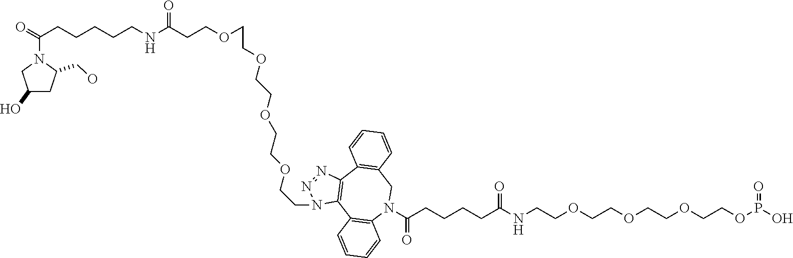 |
| Q327 (DBCO- azide 5′-3′ link (Q8 based)) | Click Q325 and L177 | 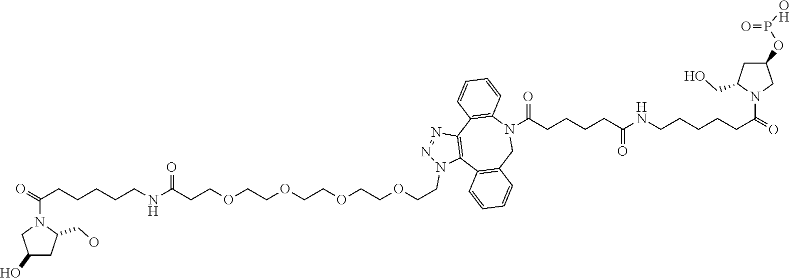 |
| Q328 (DBCO- azide 5′-5′ link (Q8 based)) | Click Q325 and Q301 | 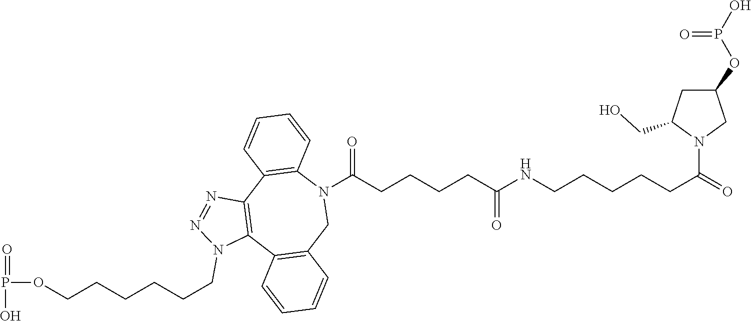 |
| Q138 Q8-C6- diphenyl- cyclo- octa (1,2,3 triazole)- C6- C6-Hyp | Click Q99 and Q136 | 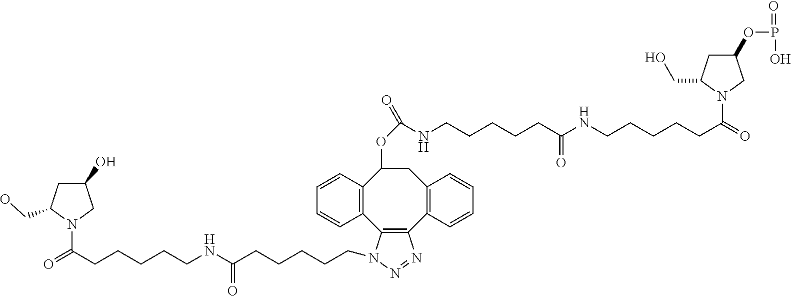 |

One exemplary cycling linking moiety contains a maleimide-thioether linkage (or thiosuccinimide linkage) formed through a cyclization procedure via a click chemistry from the thiol-maleimide addition reaction. As discussed above in Scheme 1, the cyclization can be formed by attaching a reactive linking moiety Q to one end of the sense (or antisense) strand, attaching another reactive linking moiety Y to the other end of the sense (or antisense) strand, and activating the reaction between Q and Y. The Q/Y pair in this case is thiol/maleimide pair. Non-limiting exemplary molecules that contain the reactive linking moiety Q/Y (in this case, thiol/maleimide functional groups) are illustrated below.

| | |
|---|---|
| Q157 N-[4-(N- maleimidomethyl)cyclohexane-1- carboxamidocaproyl)prolinol-4- phosphate (Hyp-C6-maleimide) | 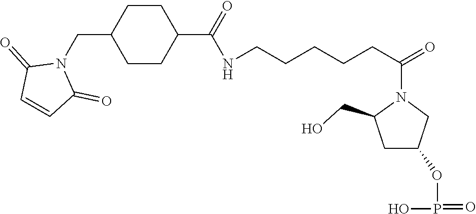 |
| Q385 N-[4-(N- maleimidomethyl)cyclohexane-1- carboxamidocaproyl)-(S)- pyrrolidin-3-ol-phosphate (Q358- maleimide) | 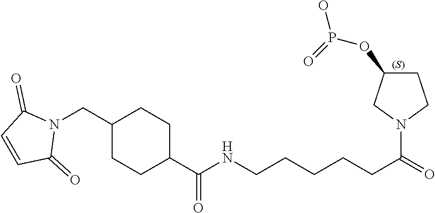 |
| Y224 2′ maleimide adenosine-3′ phosphate (attached via (Aah)) | 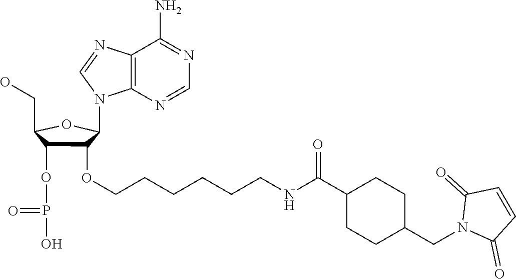 |
| Q5 6-(2-mercapto-acetylamino)-hexyl phosphate | 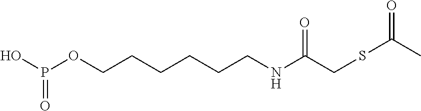 |
| Q51 6- hydroxyhexyldithiohexyl phosphate (Thiol-Modifier C6 S-S Glen Res. 10-1936) | 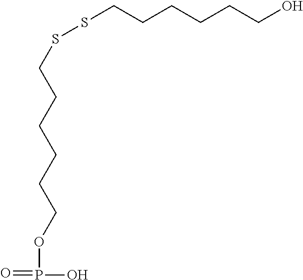 |
| Q66 6-mercaptohexylphosphate (Glen Res. 10-1936) | 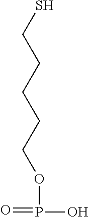 |
| Q82 1-thiohexylphosphate (after cleavage of Thiol-Modifier C6 S-S Glen Res. 10-1936) | 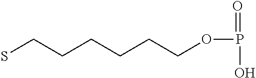 |
| Q332 6-(3-mercaptopropanamido)hexyl phosphate (5′-aminomodifier with thiol for conjugation) | 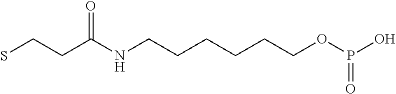 |
| (dTbm) S-isobutyryl-(2- mercaptoethylglycol)-2′- deoxythymidine-3′-phosphotriester (dT-BMEG) | 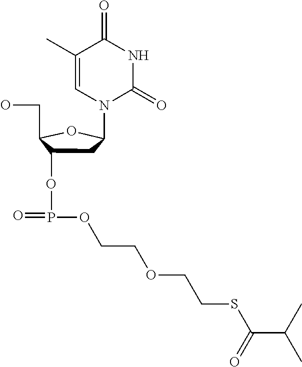 |
| (ubm) S-isobutyryl-(2- mercaptoethylglycol)-2′O- methyluridine-3′-phosphotriester (u-BMEG) | 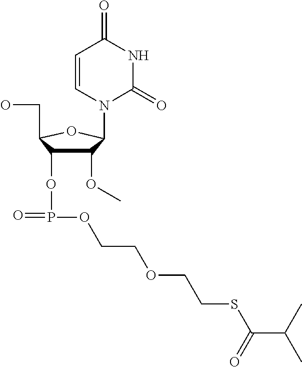 |
| (Ufbm) S-isobutyryl-(2- mercaptoethylglycol)-2′- fluorouridine-3′-phosphotriester (fU-BMEG) | 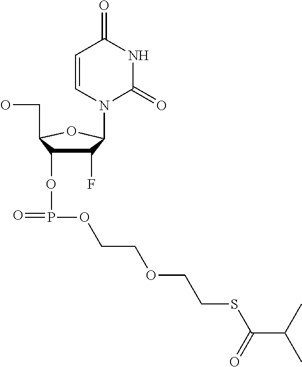 |
| Y138 2′-O-[{2-(butyldisulfaneyl)-2- methylpropyl}carbamate]-uridine- 3′-phosphate | 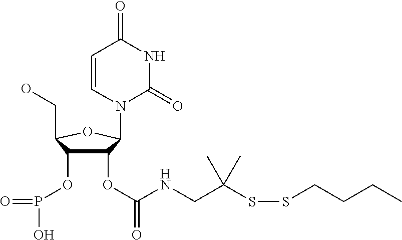 |

The sense strand can be at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. In one embodiment, the sense strand is at least 20 nucleotides in length. In one embodiment, the sense strand is at least 40 nucleotides in length.

The antisense strand can be at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length.

The antisense strand is annealed with the sense strand to form at least a partial duplex region. In some embodiments, one or more sense nucleotide sequences are annealed with the antisense strand. In some embodiments, at least one sense nucleotide sequence is not annealed with the antisense strand.

In some embodiments, the sense nucleotide sequence not annealed with the antisense strand can be a single-stranded oligonucleotide, such as an antisense oligonucleotide (ASO), an antimiR (antagomir) oligonucleotide, or a single-stranded siRNA (ss-siRNA) oligonucleotide.

In some embodiments, a duplex region is formed between the sense strand and antisense strand at least at the seed region of the antisense strand (i.e., at positions 2-8 of the 5′-end of an antisense nucleotide sequence).

Increasing the length of the sense strand, therefore the length of the duplex region, can have an impact on melting temperature of the sciRNA and can increase the thermal stability of the sciRNA duplex.

Increasing the length of the sense strand can be achieved by using a single sense nucleotide sequence, or by having more than one sense nucleotide sequences in the sense strand.

In some embodiments, the sense strand can have a long circular sense nucleotide sequence, having at least 20 nucleotides in length, at least 25 nucleotides in length, or at least 30 nucleotides in length, for instance, having 20 to 45 nucleotides in length, or 30 to 45 nucleotides in length.

In some embodiments, the antisense strand comprises at least one antisense nucleotide sequence. The at least one antisense nucleotide sequence has about 20 to about 45 nucleotides in length.

In one embodiment, a long circular sense nucleotide sequence is annealed with an antisense strand having about 19 to about 23 nucleotides in length, complementary to a target mRNA transcript nucleotide sequence. In one embodiment, a long circular sense nucleotide sequence is annealed with two or more antisense nucleotide sequences having about 19 to about 23 nucleotides in length, complementary to two or more target mRNA transcript nucleotide sequences.

The long circular sense nucleotide sequence may be a substrate cleavable by DICER.

In some embodiments, the sense strand can comprise at least two sense nucleotide sequences. Each of the at least two sense nucleotide sequences may have about 18 to about 28 nucleotides in length. The total length of the sense strand can be at least 40 nucleotides. In one embodiment, the sense strand is a 42-nucleotide circular sense strand. In another embodiment, the sense strand is a 45-nucleotide circular sense strand.

In some embodiments, the antisense strand comprises at least two antisense nucleotide sequences. The antisense strand comprises at least 40 nucleotides in length. Each of the at least two antisense nucleotide sequences has about 18 to about 28 nucleotides in length.

For instance, the sense strand may contain two sense nucleotide sequences, forming a bis-sciRNA. The sense strand can contain two symmetrical nucleotide sequences, or two asymmetrical nucleotide sequences. In the symmetrical scenario, the circular sense strand (e.g., each may have about 19 to about 23 nucleotides in length, e.g., 21 nucleotides in length) can be annealed with two identical antisense nucleotide sequences (e.g., each may have 21 nucleotides in length), targeting the same mRNA transcript nucleotide sequence. In the asymmetrical scenario, the circular sense strand (e.g., each may have about 19 to about 23 nucleotides in length, e.g., 21 nucleotides in length) can be annealed with two different antisense nucleotide sequences (e.g., each may have 23 nucleotides in length), targeting two different mRNA transcript nucleotide sequences.

Exemplary circular sense strands and circular sciRNA are shown in Schemes 1A-1C. Schemes 1A and 1B each illustrate a circular sense strand containing two symmetrical (Scheme 1A) or asymmetrical (Scheme 1B) sense nucleotide sequences (with a total length of the sense strand of 42-45 nucleotides). The two sense nucleotide sequences are connected by a nucleotide-based or non-nucleotide based linker (tether). Scheme 1C illustrates a circular sense strand containing a long dicer-cleavable sense nucleotide sequence (e.g., 30 to 45 nucleotides), annealed with a shorter antisense nucleotide sequence (e.g., 19-23 nucleotides). The circular or substantially circular structure of the sense strand may be formed by click chemistry. The cycling linking moiety Z contains the combination of one or more of phosphate linkage, alkyl linkage, triazole linkage, amide linkage, and pyrrolidinyl cyclic group, with or without a ligand (L) carried by the cyclic group.

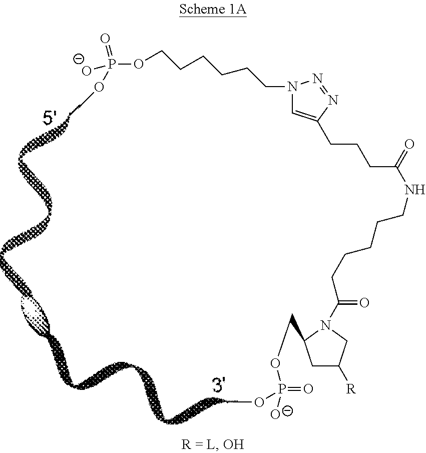

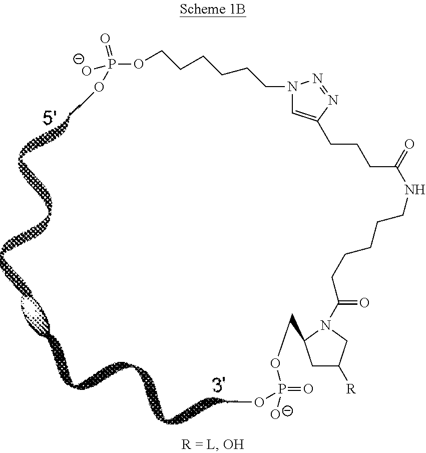

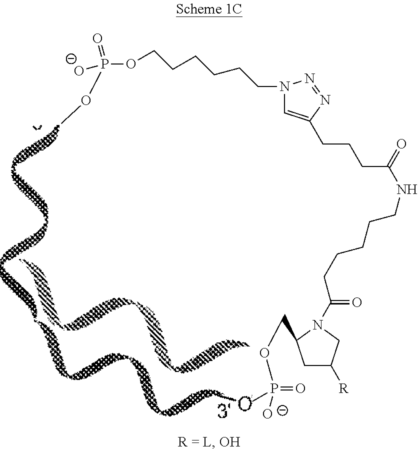

Additional exemplary circular sense strands and circular sciRNA are shown in Schemes 2A-2C, Schemes 3A-3C, Schemes 4A-4C, Schemes 5A-5C, Schemes 6A-6C, Schemes 7A-7C, and Schemes 8A-8C, illustrating various cyclization reactions and cycling linking moieties. The sense and antisense strands in these schemes reflect those in Schemes 1A-1C. The cyclization reaction in these schemes are different than those in Schemes 1A-1C. For instance, in Schemes 2A-2C, the cyclization is by amide formation. The cycling linking moiety Z contains the combination of one or more of phosphate linkage, alkyl linkage, amide linkage, and a pyrrolidinyl cyclic group, with or without a ligand (L) carried by the cyclic group. In Schemes 3A-3C, the cyclization is by disulfide formation. The cycling linking moiety Z contains the combination of one or more of phosphate linkage, alkyl linkage, disulfide linkage, amide linkage, and pyrrolidinyl cyclic group, with or without a ligand (L) carried by the cyclic group. In Schemes 4A-4C, the cyclization is by click chemistry. The cycling linking moiety Z contains the combination of one or more of phosphate linkage, alkyl linkage, triazole linkage, amide linkage, and PEG linkage. In Schemes 5A-5C and 6A-6C, the cyclization is by oxime formation. The cycling linking moiety Z contains the combination of one or more of phosphate linkage, alkyl linkage, oxime linkage (aldoxime or ketoxime), amide linkage, and pyrrolidinyl cyclic group, with or without a ligand (L) carried by the cyclic group. In Schemes 7A-7C and 8A-8C, the cyclization is by hydrazone formation. The cycling linking moiety Z contains the combination of one or more of phosphate linkage, alkyl linkage, hydrazo linkage, amide linkage, and pyrrolidinyl cyclic group, with or without a ligand (L) carried by the cyclic group.

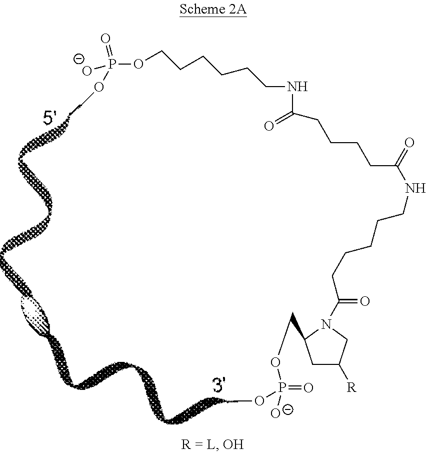

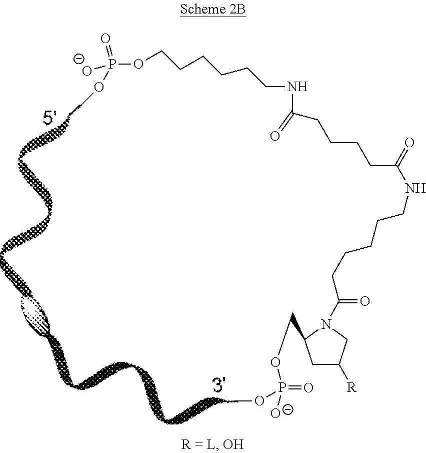

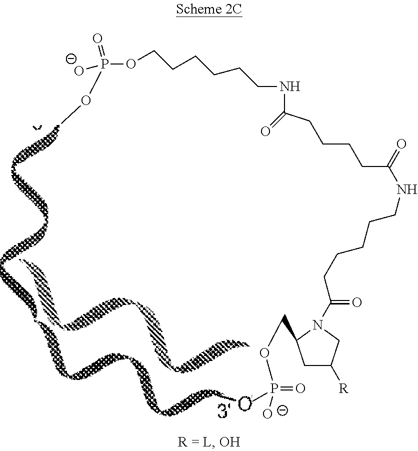

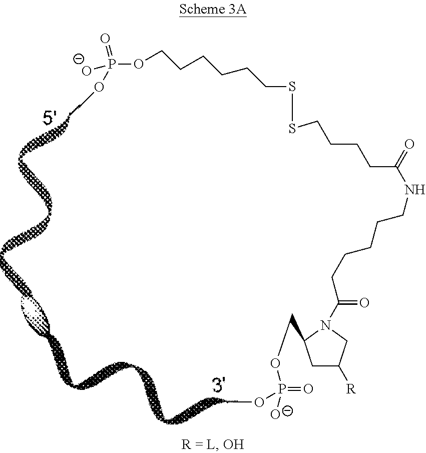

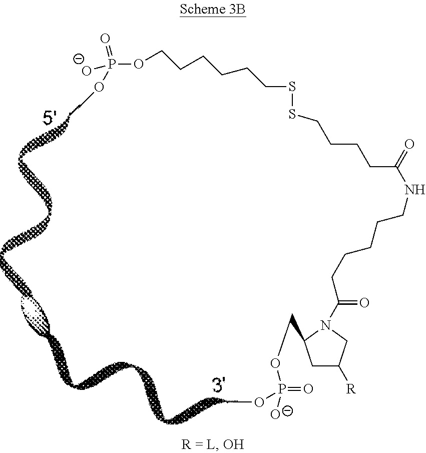

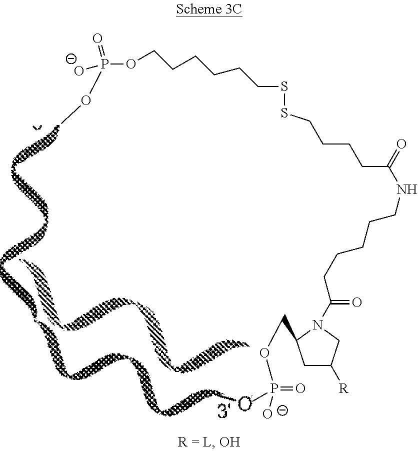

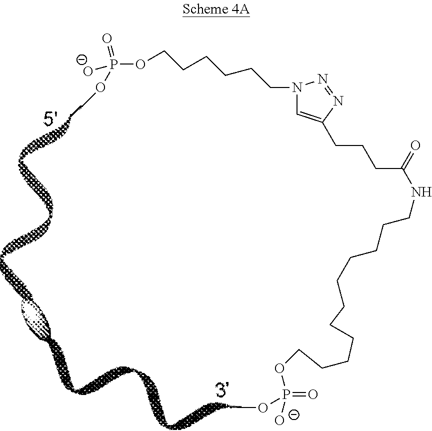 [SMILES: COP(=O)([O-])OCCCCCCCCCCCNC(=O)CCCc1cn(CCCCCCOP(=O)([O-])OC)nn1]

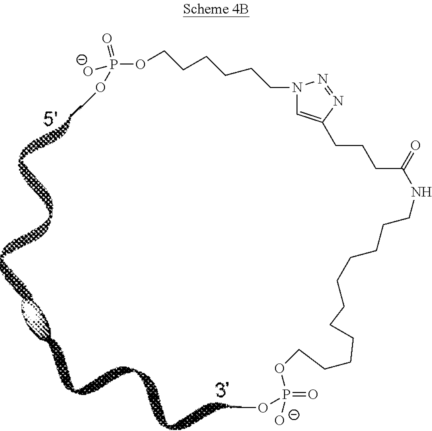 [SMILES: COP(=O)([O-])OCCCCCCCCCCCNC(=O)CCCc1cn(CCCCCCOP(=O)([O-])OC)nn1]

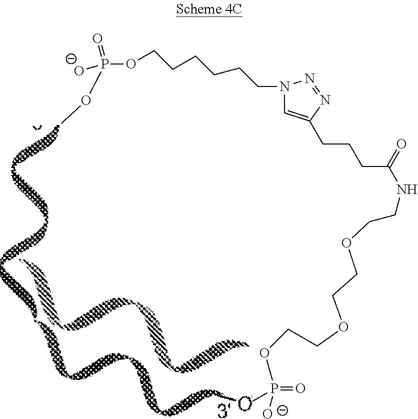 [SMILES: COP(=O)([O-])OCCCCCCn1cc(CCCC(=O)NCCOCCOCCOP(C)(=O)[O-])nn1]

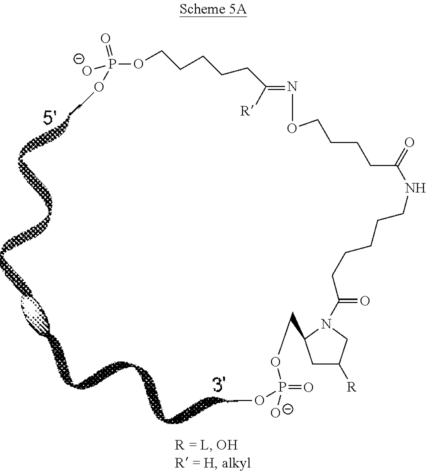

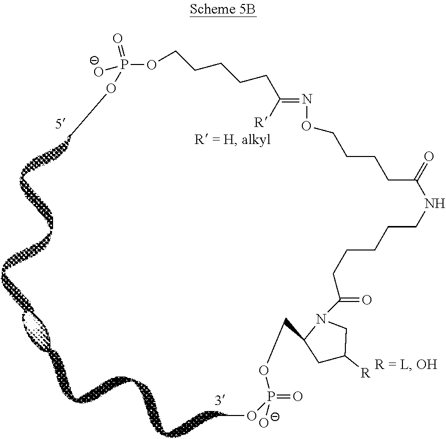

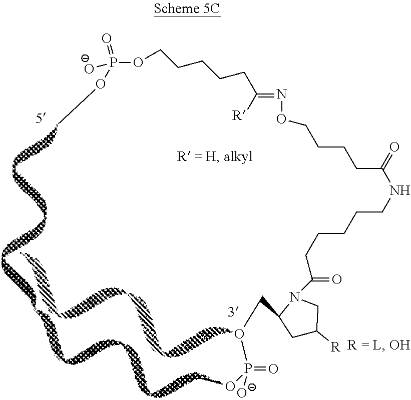

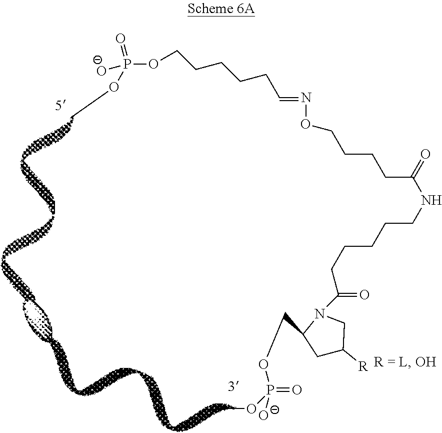

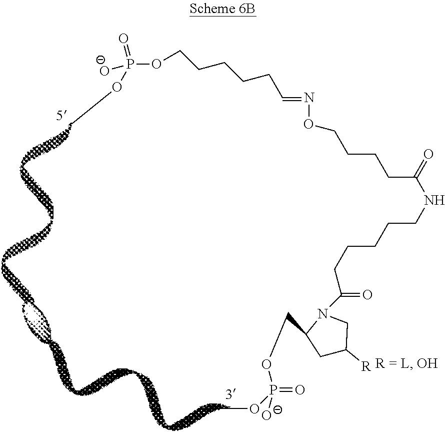

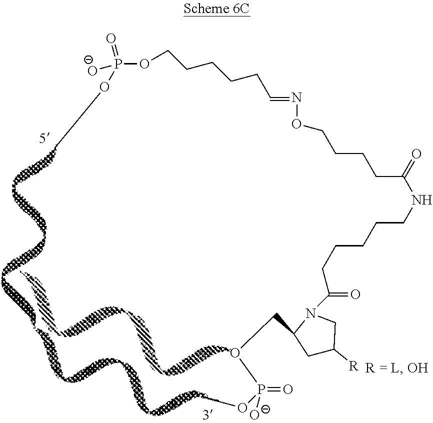

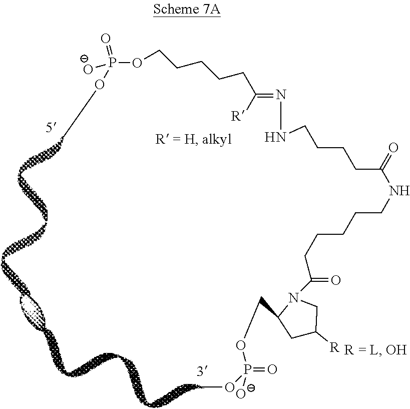

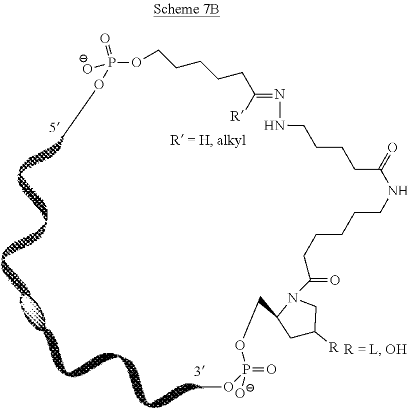

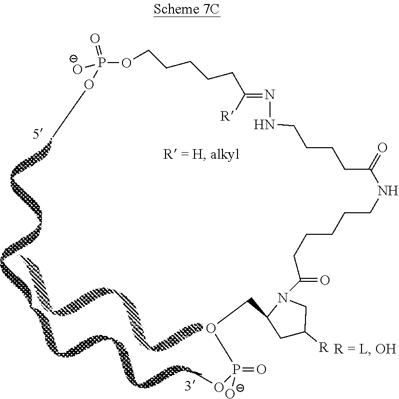

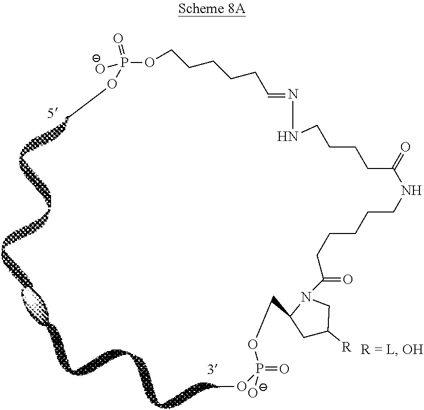

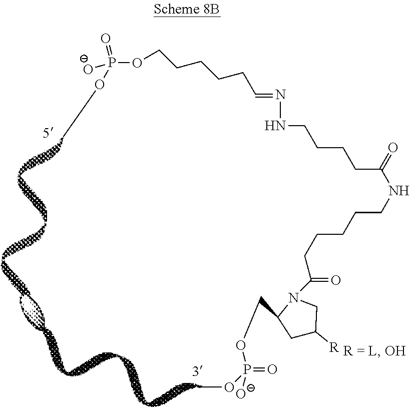

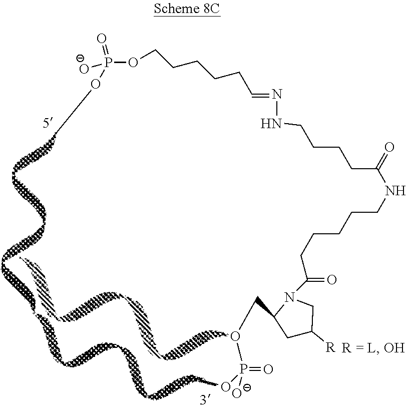

The above examples shown in Schemes 1A-1C to Schemes 8A-8C are for illustrative purpose only. The cyclization reactions and cycling linking moieties illustrated for cyclization of the sense nucleotide sequences of the sense strand would be applicable to the cyclization of the antisense nucleotide sequences of the antisense strand.

### Linkers/Tethers

Linkers/Tethers may be contained in the sense strand or antisense strand to connect two sense nucleotide sequences or antisense nucleotide sequences, or as part of the cycling linking moiety of the circular sence strand. Linkers/tethers can also be used to connect the ligand to the scriRNA, e.g., via a carrier. The terms “linker,” “linkage,” “linking group,” “tether” can be used interchangeably.

Linkers in the sense strand or antisense strand may be a nucleotide-based or non-nucleotide-based linker. The linker may be a stable linker that is stable in a biological fluid (e.g., in plasma or artificial cerebrospinal fluid). Alternatively, the linker may be a cleavable linker.

Linkers/tethers may be connected to a ligand at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the ligand. Non-limited examples of linkers/tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)—(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O)(LIGAND); TAP-(CH₂)ₙ—C(O)O(LIGAND); TAP-(CH₂)ₙLIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O)OC₆F₅; or TAP-NR″″(CH₂)ₙC(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

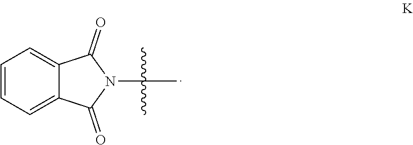 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

The cleavable linker may be cleavable in various tissue and cell structures, e.g., in liver homogenates, liver tritosomes, liver lysosomes, liver cytosol, brain homogenates, brain tritosomes, brain lysosomes, or brain cytosol.

Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers/Tethers

The linkers can also include biocleavable linkers that are nucleotide and non-nucleotide linkers, or combinations thereof, that connect two parts of a molecule. For example, a biocleavable linker may connect one or both strands of two individual siRNA molecule, to generate a bis(siRNA). In some embodiments, mere electrostatic or stacking interaction between two individual siRNAs can represent a linker.

The non-nucleotide linkers include tethers or linkers derived from monosaccharides, disaccharides, oligosaccharides, and derivatives thereof, aliphatic, alicyclic, hetercyclic, and combinations thereof.

In some embodiments, at least one of the linkers (tethers) is a bio-clevable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

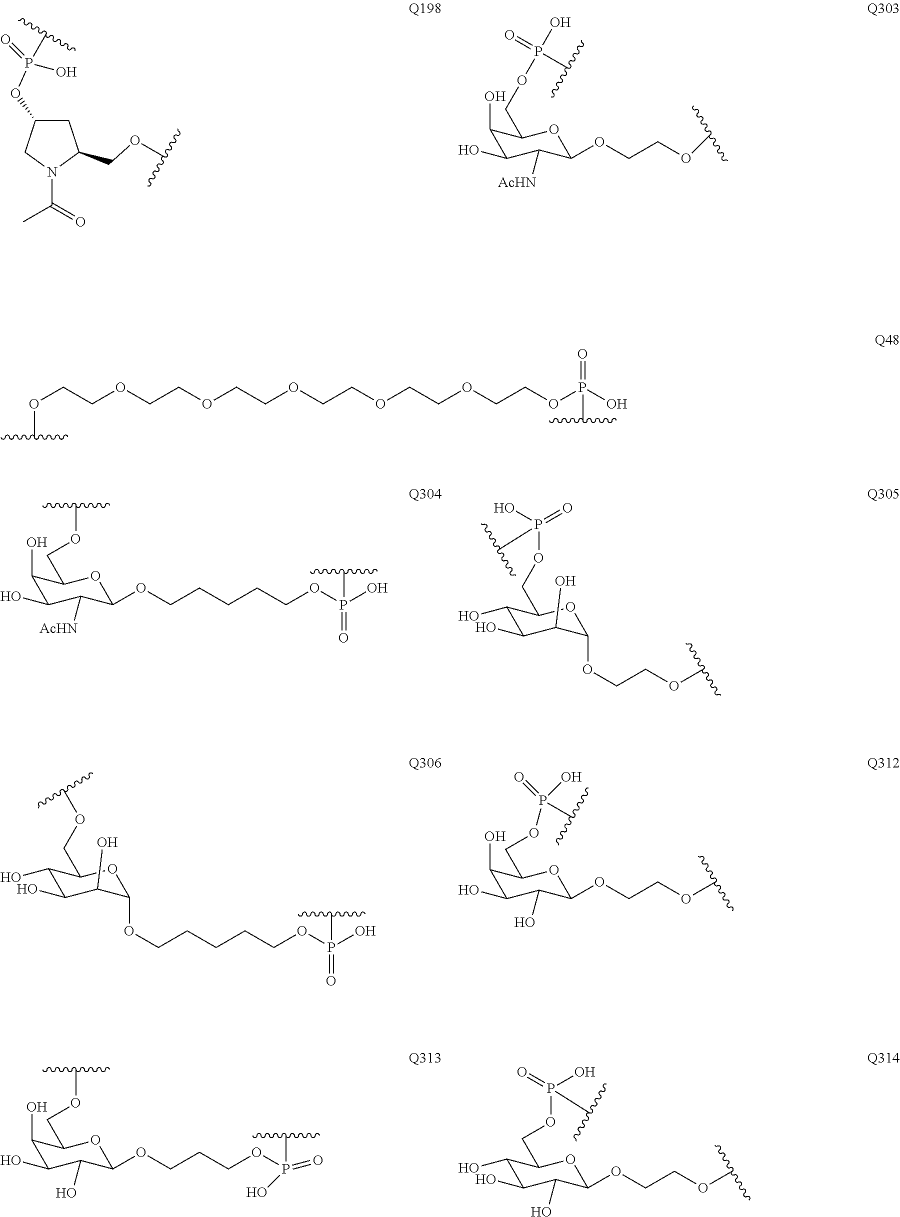

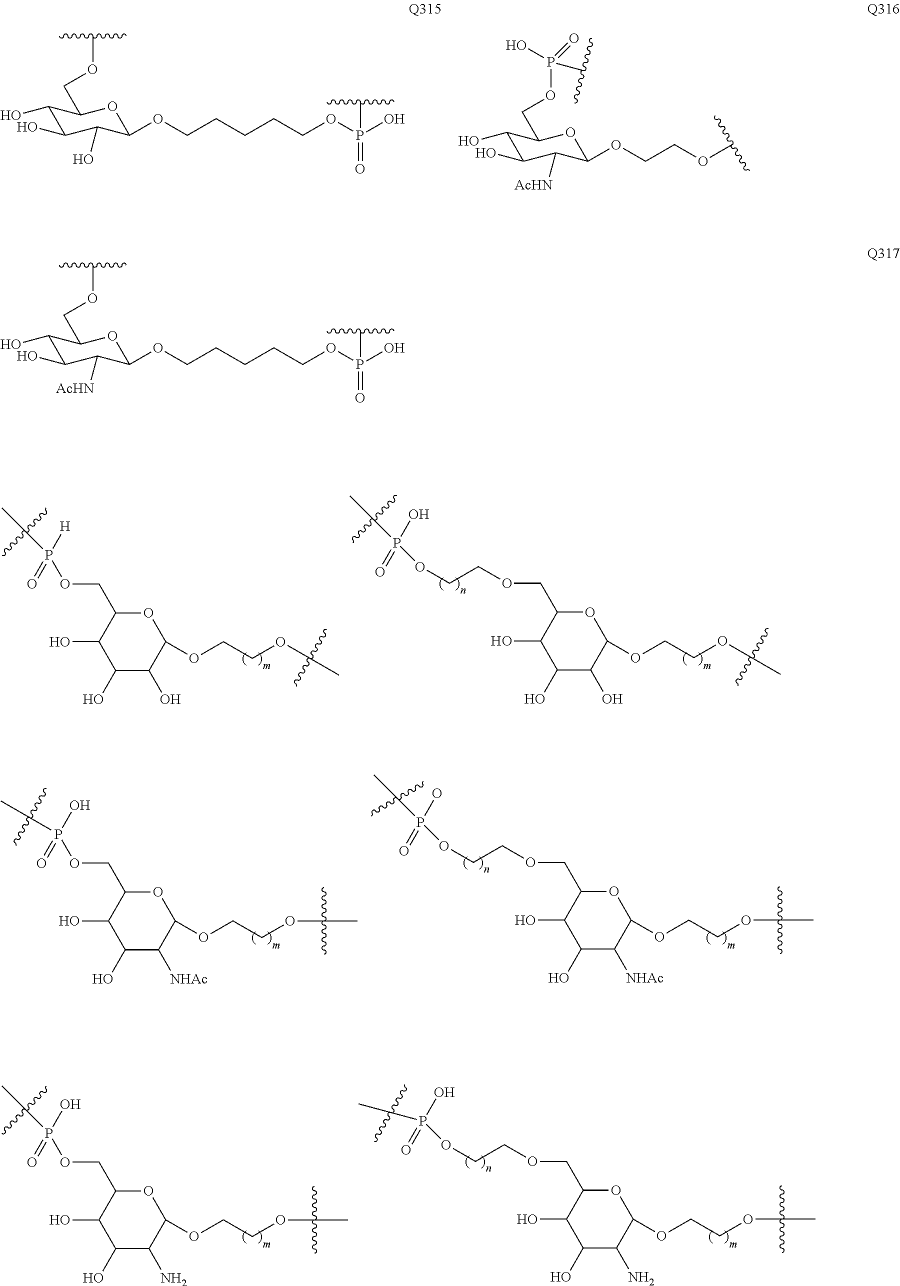

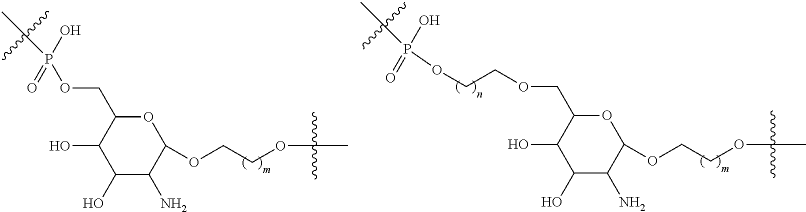

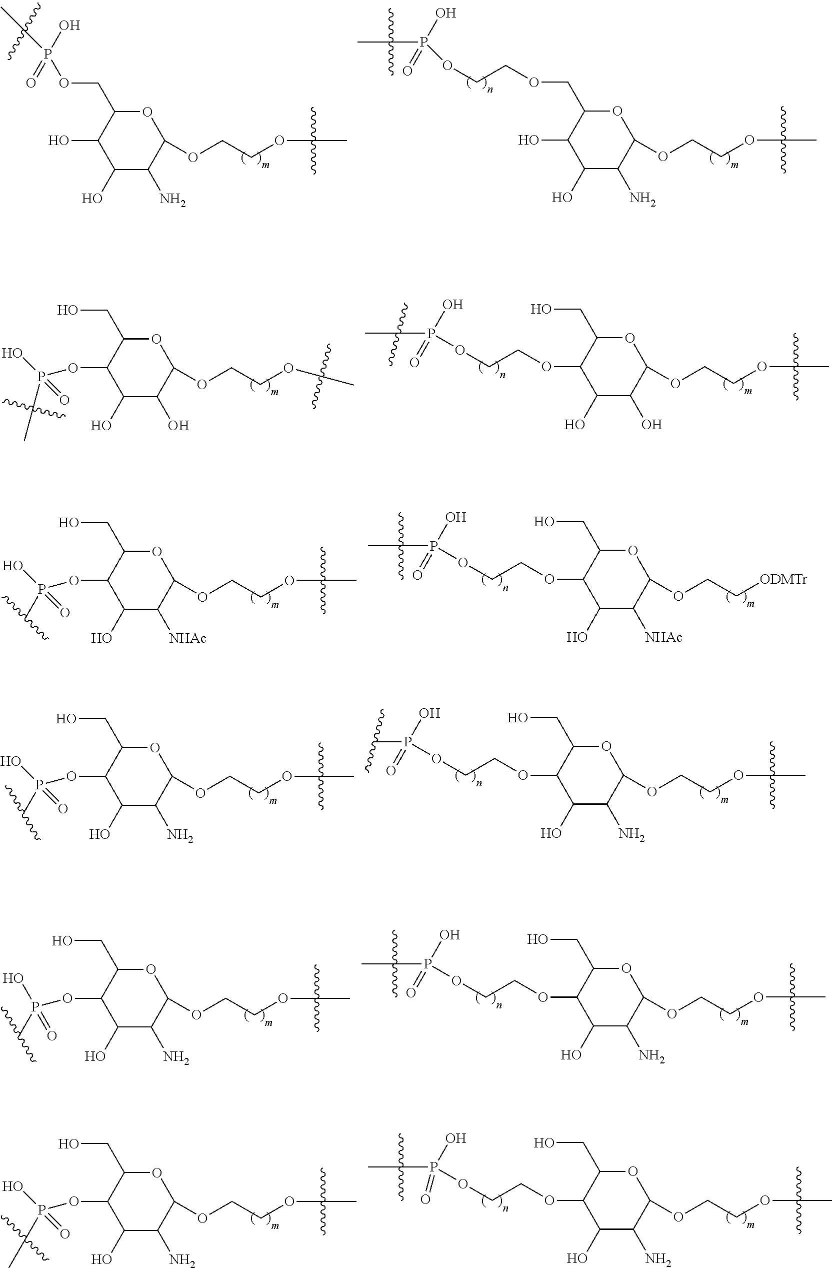

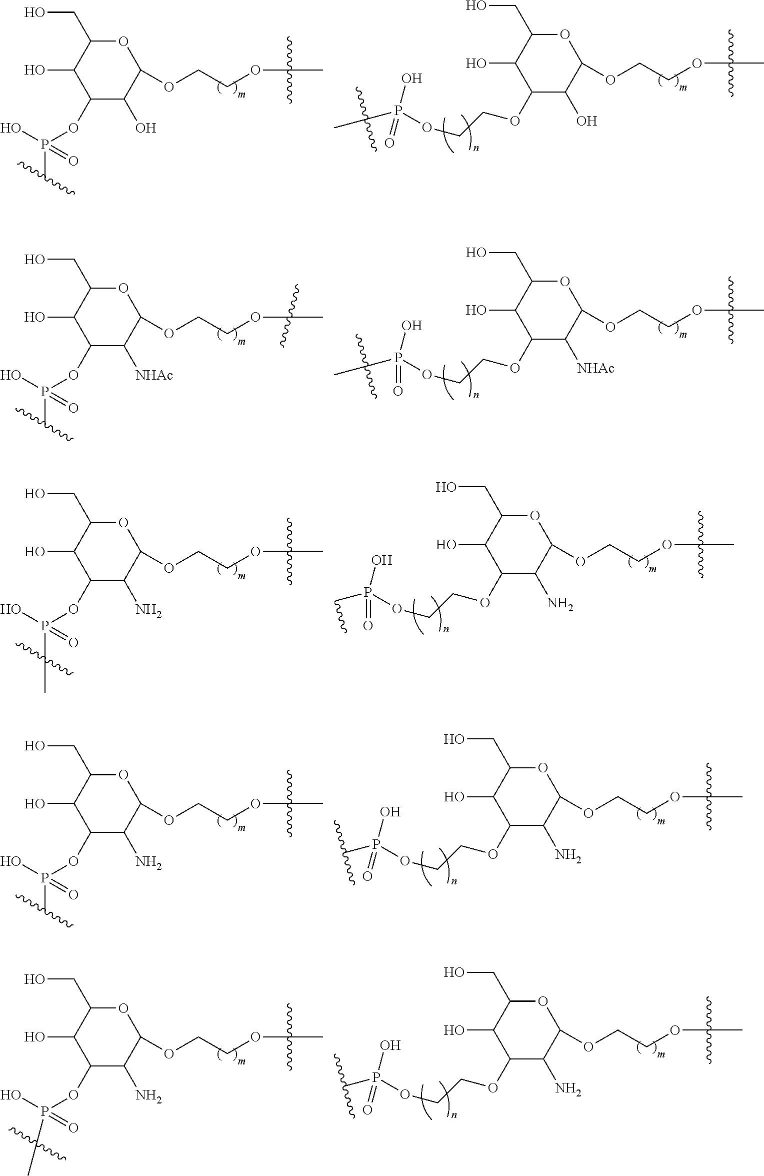

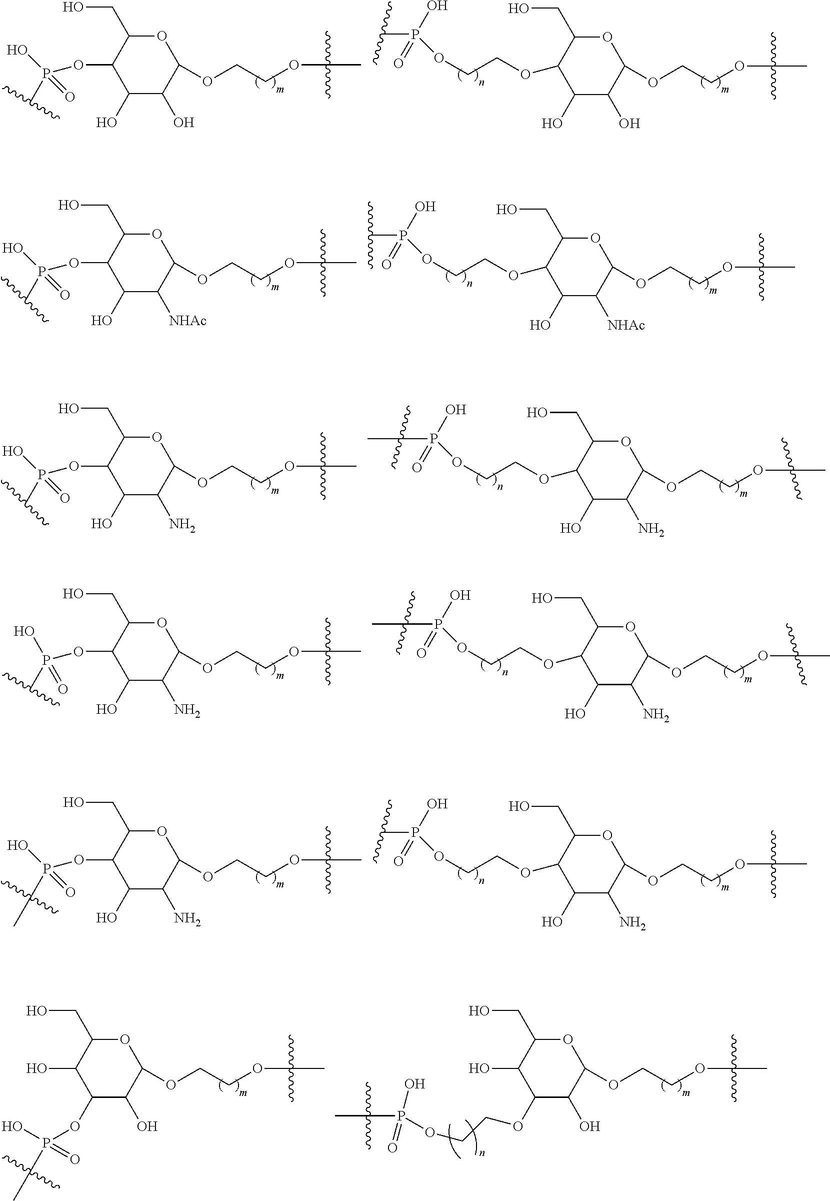

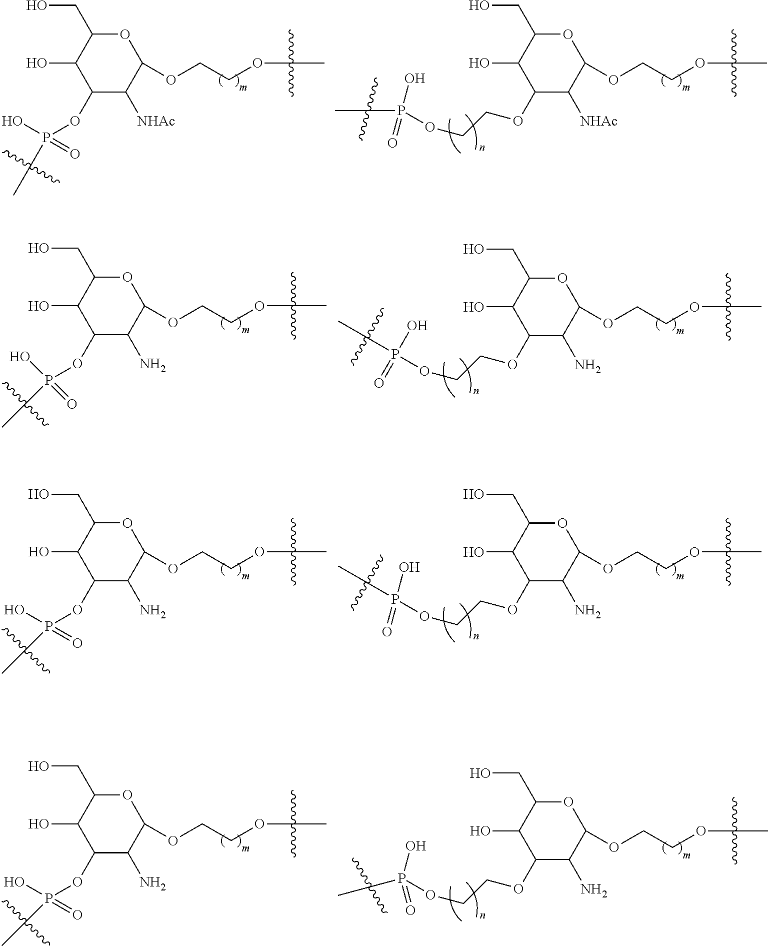

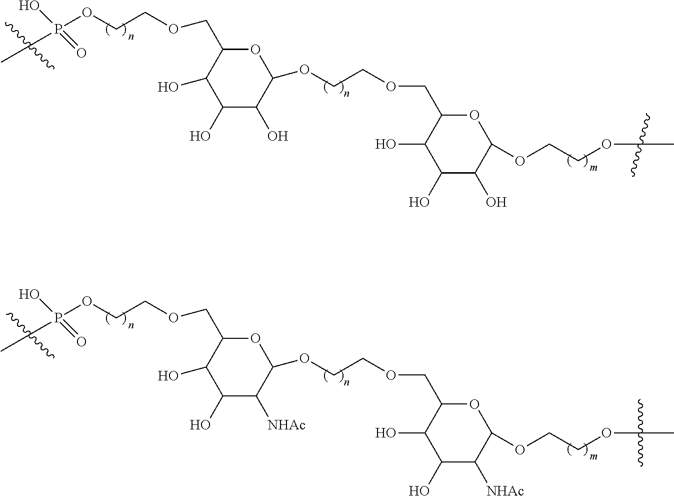

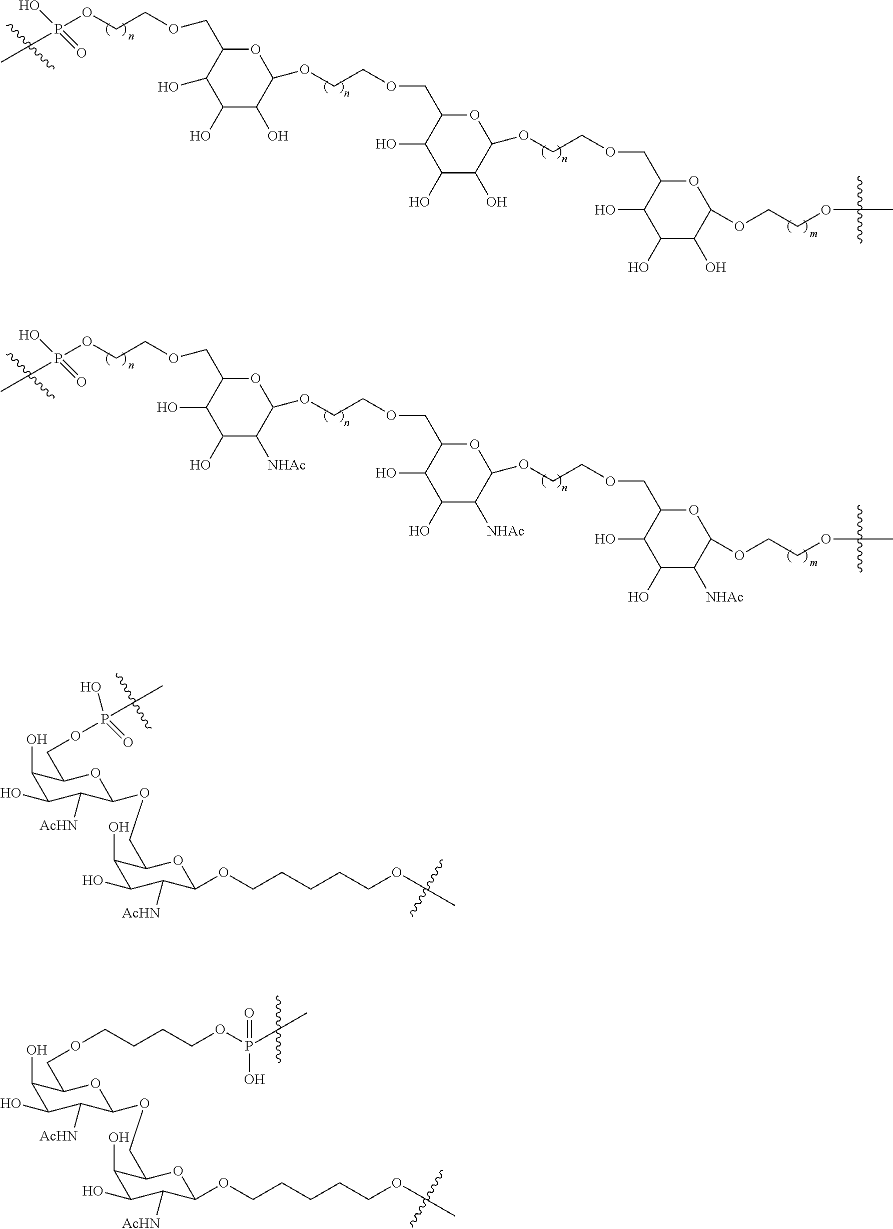

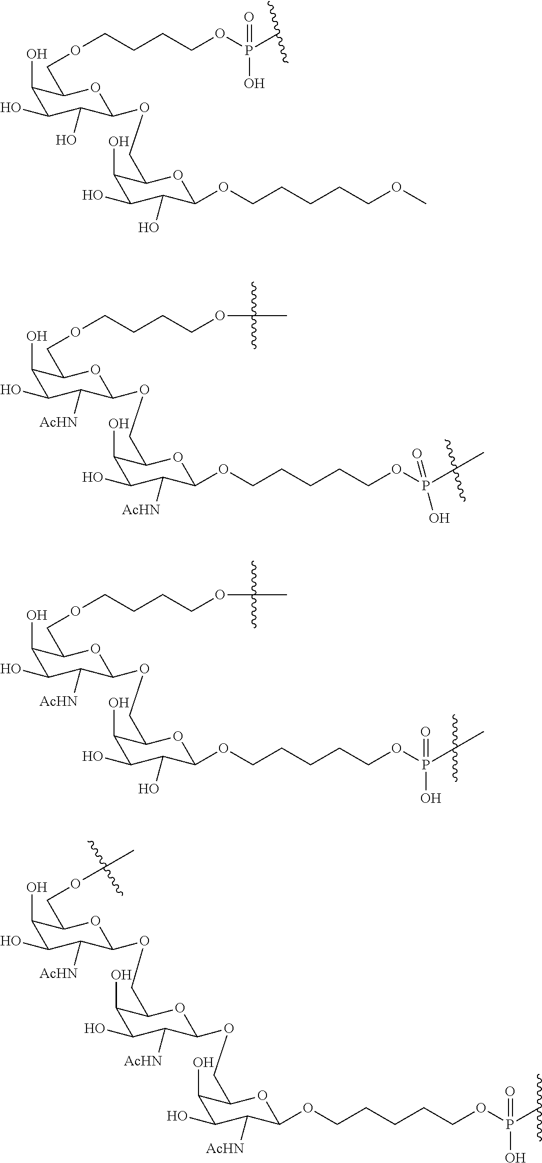 [SMILES: *P(=O)(O)OCCCCOCC1OC(OCC2OC(OCCCCCOC)C(O)C(O)C2O)C(O)C(O)C1O ; *OCCCCOCC1OC(OCC2OC(OCCCCCOP(*)(=O)O)C(NC(C)=O)C(O)C2O)C(NC(C)=O)C(O)C1O ; *OCCCCOCC1OC(OCC2OC(OCCCCCOP(*)(=O)O)C(O)C(O)C2O)C(NC(C)=O)C(O)C1O ; *OCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O]

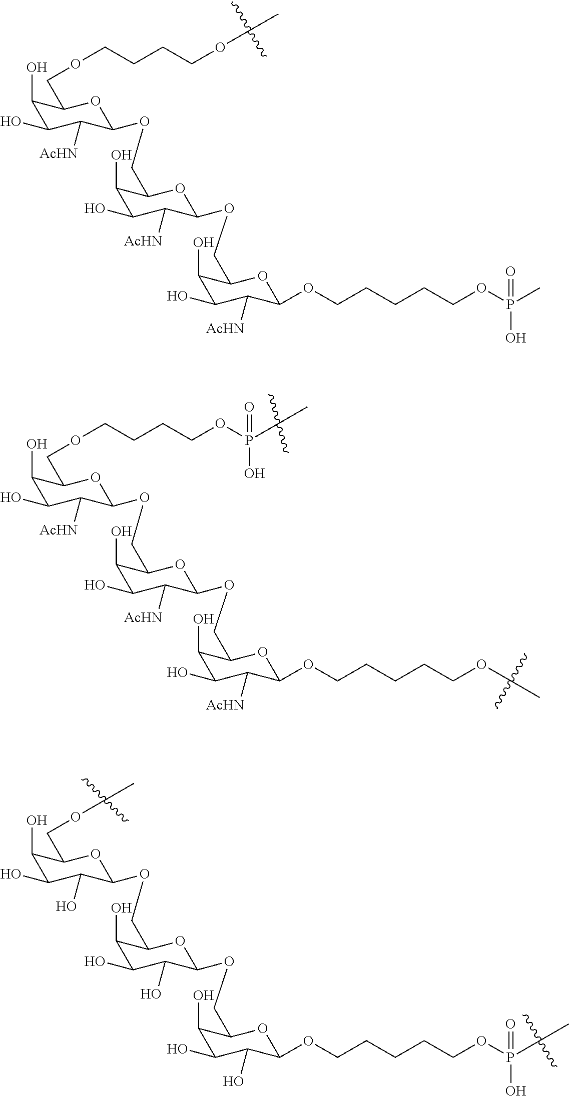 [SMILES: *OCCCCOCC1OC(OCC2OC(OCC3OC(OCCCCCOP(C)(=O)O)C(NC(C)=O)C(O)C3O)C(NC(C)=O)C(O)C2O)C(NC(C)=O)C(O)C1O ; *OCCCCCOC1OC(COC2OC(COC3OC(COCCCCOP(*)(=O)O)C(O)C(O)C3NC(C)=O)C(O)C(O)C2NC(C)=O)C(O)C(O)C1NC(C)=O ; *OCC1OC(OCC2OC(OCC3OC(OCCCCCOP(*)(=O)O)C(O)C(O)C3O)C(O)C(O)C2O)C(O)C(O)C1O]

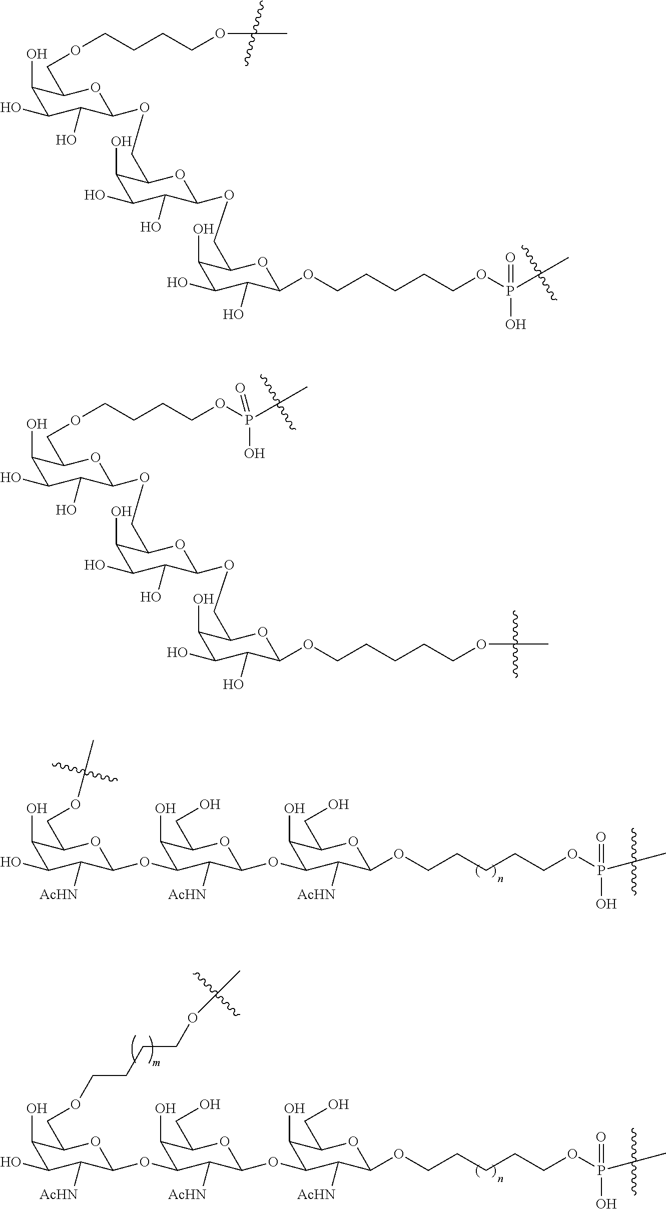

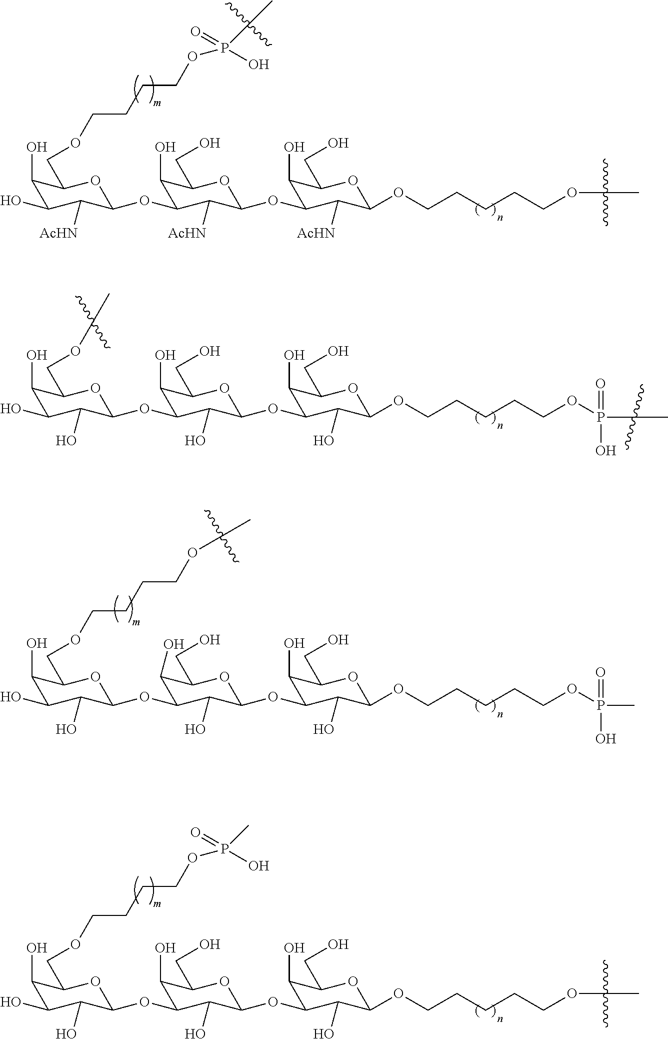

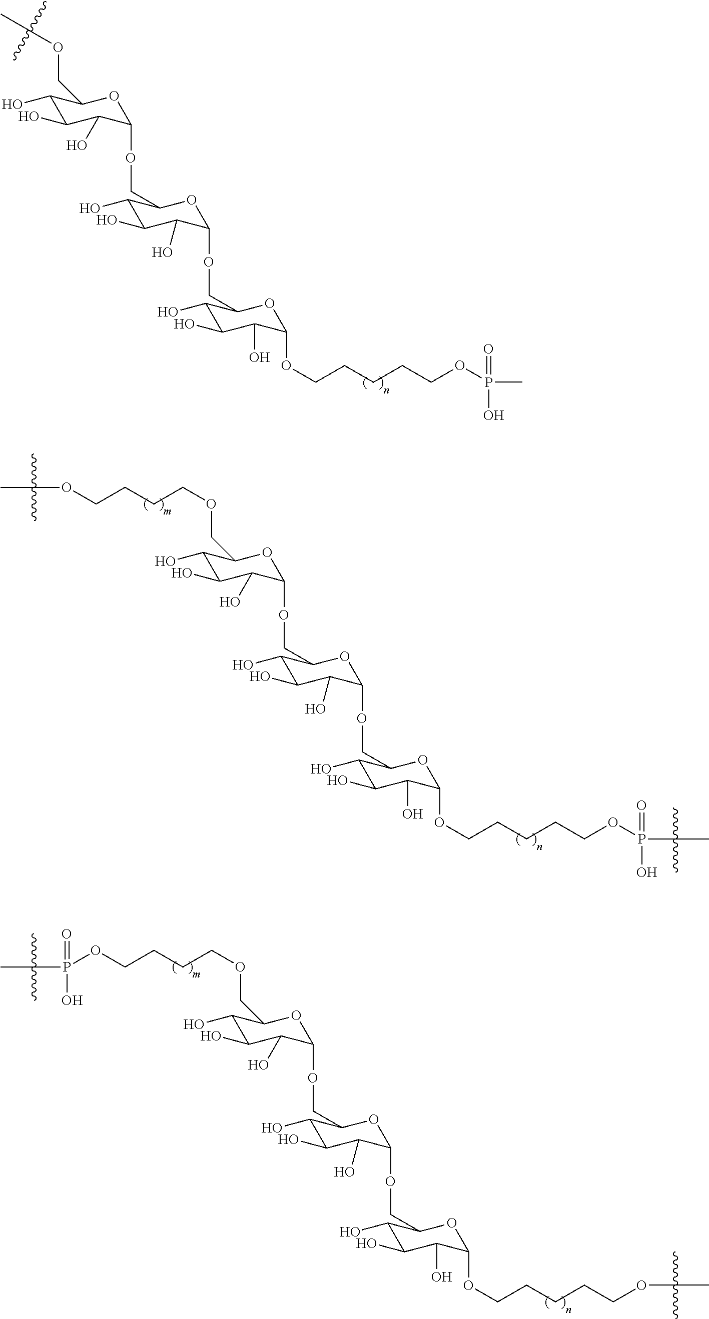

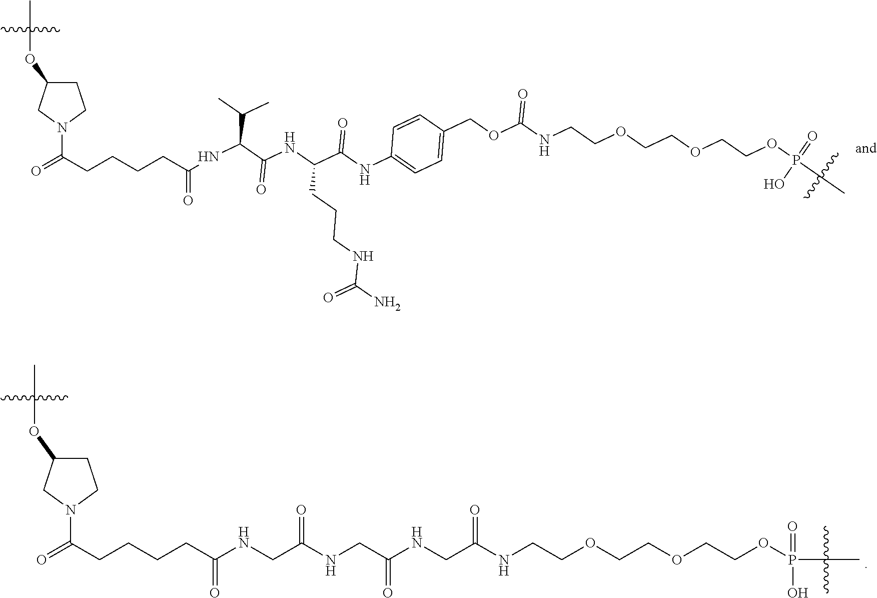 [SMILES: *O[C@H]1CCN(C(=O)CCCCC(=O)N[C@H](C(=O)N[C@@H](CCCNC(N)=O)C(=O)Nc2ccc(COC(=O)NCCOCCOCCOP(*)(=O)O)cc2)C(C)C)C1 ; *O[C@H]1CCN(C(=O)CCCCC(=O)NCC(=O)NCC(=O)NCC(=O)NCCOCCOCCOP(*)(=O)O)C1]

More discussion about the biocleavable linkers may be found in WO2018136620, the content of which is incorporated herein by reference in its entirety.

### Carriers

In certain embodiments, the cycling linking moiety of the sense (or antisense) strand contains one or more carriers that carry one or more ligands and serve to conjugate the ligand(s) to the sciRNA. In some embodiments, the carrier may replace one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalinyl. In one embodiment, the acyclic group is a moiety based on a serinol backbone or a diethanolamine backbone.

In some embodiments, the carrier replaces one or more nucleotide(s) in the internal position(s) of the sciRNA agent.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand. The ligand can be directly attached to the carrier or indirectly attached to the carrier by an intervening linker/tether, as described above.

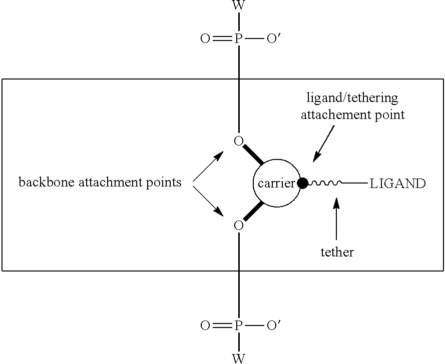

The ligand-conjugated monomer subunit may be the 5′ or 3′ terminal subunit of the sciRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an sciRNA agent.

Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰, is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

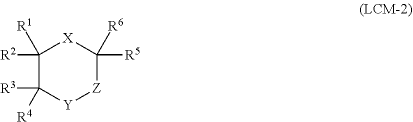

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or
Si(X^{5′})(X^{5″})(X^{5″}) in which (X^{5′}), (X^{5″}), and (X^{5″″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹², or X is N(CO)R⁷ or NR⁷, Y is NR⁸, and Z is CR¹¹R¹²; or X is N(CO)R⁷ or NR⁷, Y is O, and Z is CR¹¹R¹², or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

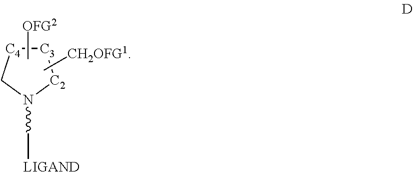

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

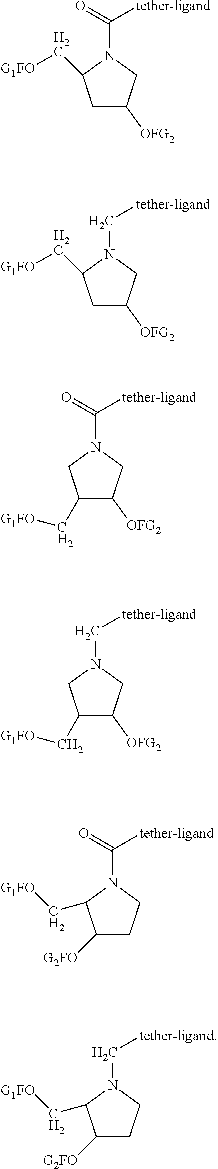

In certain embodiments, the carrier may be based on the piperidine ring system (E), e.g., X is N(CO)R⁷ or NR⁷, Y is CR⁹R¹⁰, and Z is CR¹¹R¹².

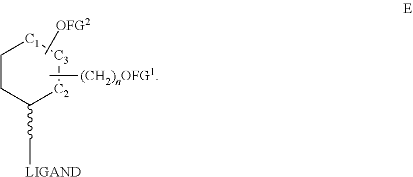

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁷, and Z is CR¹¹R¹², or the morpholine ring system (G), e.g., X is N(CO)R⁷ or NR⁷, Y is O, and Z is CR¹¹R¹².

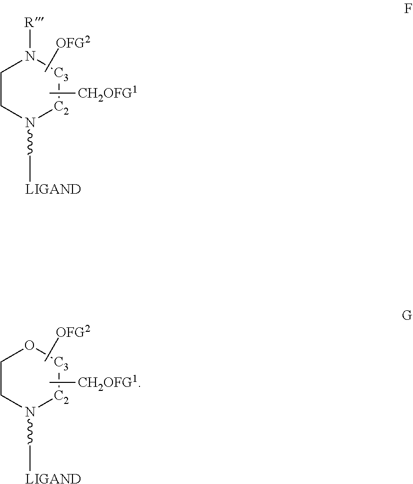

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

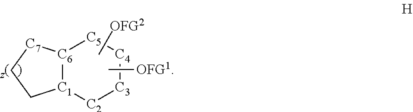

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

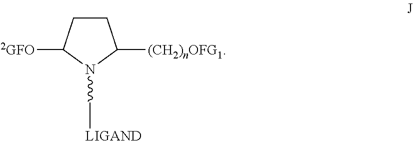

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

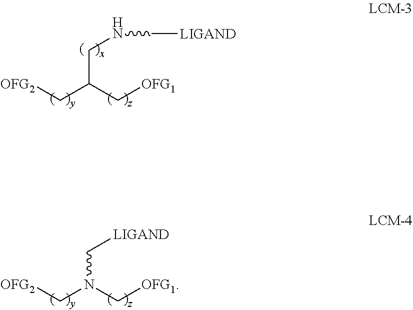

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the sciRNA agent comprises one or more ligands conjugated to the 5′ end of a sense nucleotide sequence or the 5′ end of an antisense nucleotide sequence.

In certain embodiments, the ligand is conjugated to the 5′-end of a nucleotide sequence of a strand via a carrier and/or linker. In one embodiment, the ligand is conjugated to the 5′-end of a nucleotide sequence of a strand via a carrier of a formula:

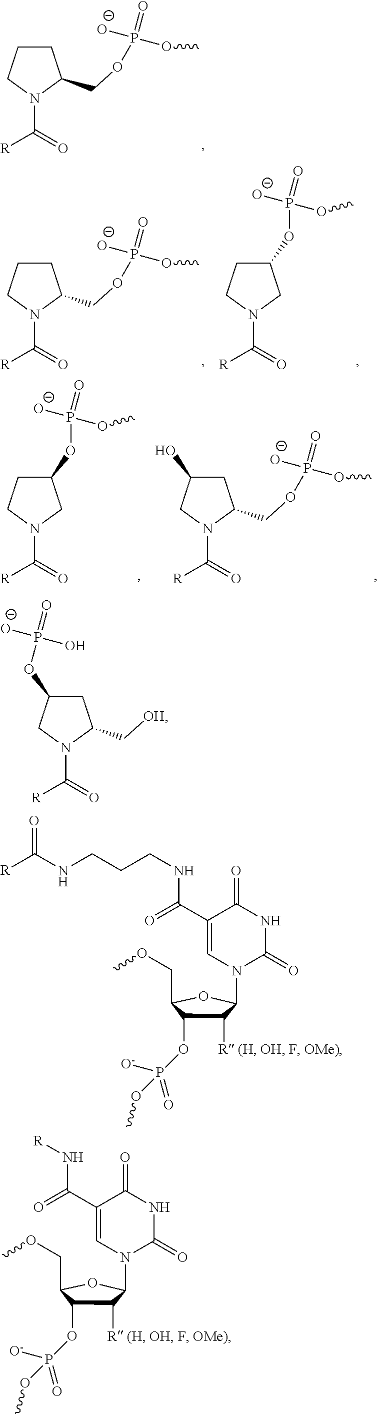

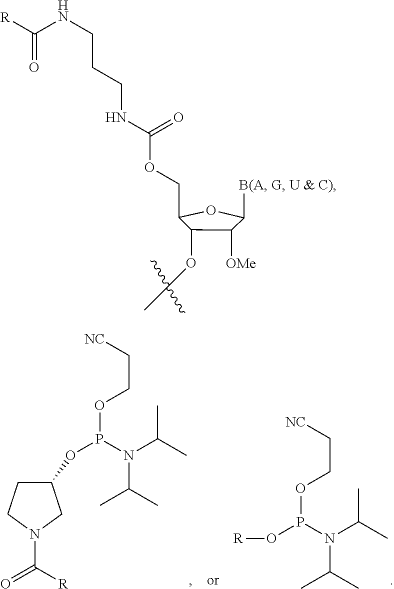

R is a ligand.

In some embodiments, the sciRNA agent comprises one or more ligands conjugated to the 3′ end of a sense nucleotide sequence or the 3′ end of an antisense nucleotide sequence.

In certain embodiments, the ligand is conjugated to the 3′-end of a nucleotide sequence of a strand via a carrier and/or linker. In one embodiment, the ligand is conjugated to the 3′-end of a nucleotide sequence of a strand via a carrier of a formula:

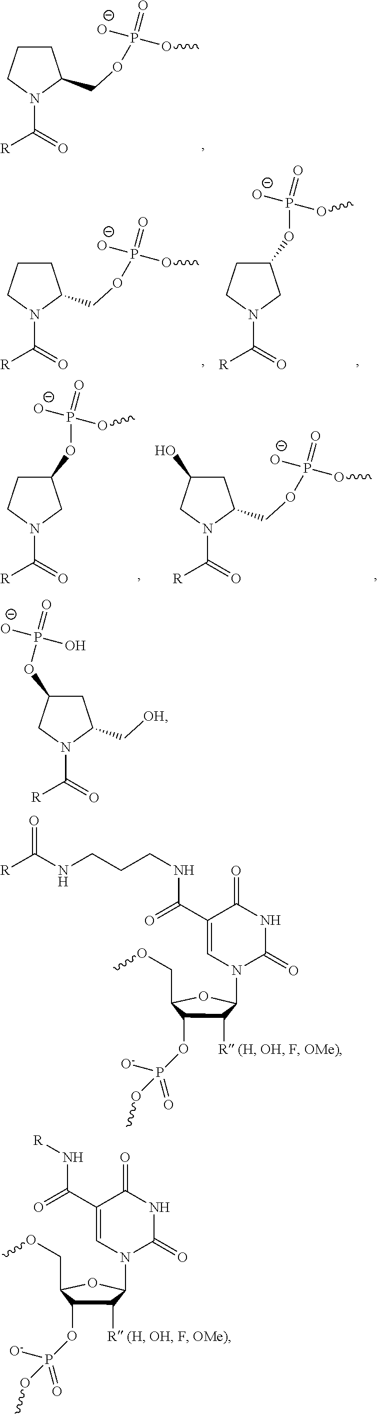

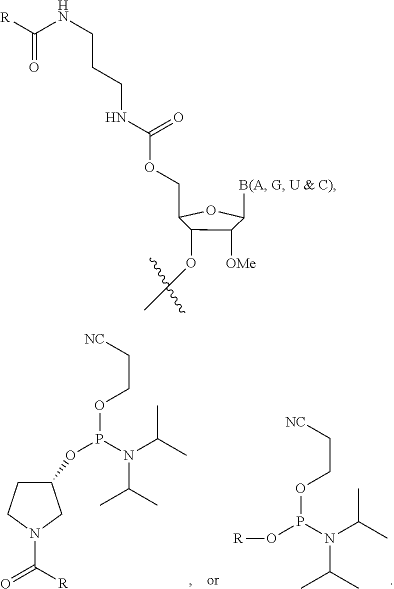

R is a ligand.

In certain embodiments, at least one of the ligands is conjugated to a strand that has a circular or substantially circular structure. In certain embodiments, at least one of the ligands is conjugated to a strand that does not have a circular or substantially circular structure. In one embodiment, at least one of the ligands is conjugated to a strand that has a circular or substantially circular structure, and at least one of the ligands is conjugated to a strand that does not have a circular or substantially circular structure.

In some embodiments, the sciRNA agent comprises one or more ligands conjugated to both ends of a sense nucleotide sequence. In some embodiments, the sciRNA agent comprises one or more ligands conjugated to both ends of an antisense nucleotide sequence.

In some embodiments, the sciRNA agent comprises one or more ligands conjugated to the 5′ end or 3′ end of a sense nucleotide sequence, and one or more ligands conjugated to the 5′ end or 3′ end of an antisense nucleotide sequence.

In some embodiments, the ligand is conjugated to a strand via one or more linkers (tethers) and/or a carrier. In one embodiment, the ligand is conjugated to a strand via one or more linkers (tethers).

In one embodiment, the ligand is conjugated to the 5′ end or 3′ end of a sense nucleotide sequence or antisense nucleotide sequence via a cyclic carrier, optionally via one or more intervening linkers (tethers).

In some embodiments, the ligand is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the ligand is conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the lipophilic moiety is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the ligand is conjugated to one or more internal positions on at least one strand, except the cleavage site region of a sense sequence, for instance, the ligand is not conjugated to positions 9-12 counting from the 5′-end of the sense sequence, for example, the ligand is not conjugated to positions 9-11 counting from the 5′-end of the sense sequence. Alternatively, the internal positions exclude positions 11-13 counting from the 3′-end of the sense sequence.

In one embodiment, the ligand is conjugated to one or more internal positions on at least one strand, which exclude the cleavage site region of an antisense sequence. For instance, the internal positions exclude positions 12-14 counting from the 5′-end of the antisense sequence.

In one embodiment, the ligand is conjugated to one or more internal positions on at least one strand, which exclude positions 11-13 on a sense sequence, counting from the 3′-end, and positions 12-14 on an antisense sequence, counting from the 5′-end.

In one embodiment, one or more ligands are conjugated to one or more of the following internal positions: positions 4-8 and 13-18 on a sense sequence, and positions 6-10 and 15-18 on an antisense sequence, counting from the 5′end of each strand.

In one embodiment, one or more ligands are conjugated to one or more of the following internal positions: positions 5, 6, 7, 15, and 17 on a sense sequence, and positions 15 and 17 on an antisense sequence, counting from the 5′end of each strand.

In some embodiments, the ligand is conjugated to a nucleobase, sugar moiety, or internucleosidic linkage of the sciRNA agent.

### Ligands

In certain embodiments, the sciRNA agent of the invention is further modified by covalent attachment of one or more conjugate groups. In general, conjugate groups modify one or more properties of the attached sciRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the sciRNA agent further comprises a targeting ligand that targets a receptor which mediates delivery to a specific CNS tissue. These targeting ligands can be conjugated in combination with the lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the sciRNA agent further comprises a targeting ligand that targets a receptor which mediates delivery to a specific ocular tissue. These targeting ligands can be conjugated in combination with the lipophilic moiety to enable specific intravitreal and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH or Cyclo(-Arg-Gly-Asp-D-Phe-Cys; LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a·helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H2A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

AALEALAEALEALAEALEALAEAAAAGGC (GALA);
AALAEALAEALAEALAEALAEALAAAAGGC (EALA);
ALEALAEALEALAEA;
GLFEAIEGFIENGWEGMIWDYG (INF-7);
GLFGAIAGFIENGWEGMIDGWYG (Inf HA-2);
GLFEAIEGFIENGWEGMIDGWYGCGLFEAIEGFIE
NGWEGMID GWYGC
(diINF-7);
GLFEAIEGFIENGWEGMIDGGCGLFEAIEGFIENGWEGMIDGGC
(diINF-3);
GLFGALAEALAEALAEHLAEALAEALEALAAGGSC (GLF);
GLFEAIEGFIENGWEGLAEALAEALEALAAGGSC (GALA-INF3);
GLF EAI EGFI ENGW EGnI DG K GLF EAI
EGFI ENGW EGnI DG
(INF-5, n is norleucine);
LFEALLELLESLWELLLEA (JTS-1);
GLFKALLKLLKSLWKLLLKA (ppTG1);
GLFRALLRLLRSLWRLLLRA (ppTG20);
WEAKLAKALAKALAKHLAKALAKALKACEA (KALA);
GLFFEAIAEFIEGGWEGLIEGC (HA);
GIGAVLKVLTTGLPALISWIKRKRQQ (Melittin);
HsWYG;
and
CHKGHC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

RQIKIWFQNRRMKWKK (penetratin);
GRKKRRQRRRPPQC (Tat fragment 48-60);
GALFLGWLGAAGSTMGAWSQPKKKRKV
(signal sequence based peptide);
LLIILRRRIRKQAHAHSK (PVEC);
GWTLNSAGYLLKINLKALAALAKKIL (transportan);
KLALKLALKALKAALKLA (amphiphilic model peptide);
RRRRRRRRR (Arg9);
KFFKFFKFFK
(Bacterial cell wall permeating peptide);
LLGDFFRKSKEKIGKEFKRIVQRIKDFLRNLVPRTES (LL-37);
SWLSKTAKKLENSAKKRISEGIAIAIQGGPR (cecropin P1);
ACYCRIPACIAGERRYGTCIYQGRLWAFCC (a-defensin);
DHYNCVSSGGQCLYSACPIFTKIQGTCYRGKAKCCK
(β-defensin);
RRRPRPPYLPRPRPPPFFPPRLPPRIPPGFPPRFPPRFPGKR-NH2
(PR-39);
ILPWKWPWWPWRR-NH2 (indolicidin);
AAVALLPAVLLALLAP (RFGF);
AALLPVLLAAP (RFGF analogue);
and
RKCRIVVIRVCR (bactenecin).

Exemplary cationic groups include, but are not limited to, protonated amino groups, derived from e.g., 0-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., 0(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the sciRNA agent. In some embodiments, the ligand can be incorporated via coupling to a “precursor” monomer after said “precursor” monomer has been incorporated into a component of the sciRNA agent. For example, a monomer having, e.g., an amino-terminated tether (i.e., having no associated ligand), e.g., monomer-linker-NH₂ can be incorporated into into a component of the sciRNA. In a subsequent operation, i.e., after incorporation of the precursor monomer into a component of the sciRNA, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the sciRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of conjugates of nucleic acids include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; contents of which are herein incorporated in their entireties by reference.

In some embodiments, the sciRNA agent further comprises one or more targeting ligands that target a liver tissue. In some embodiments, at least one of the targeting ligands is a carbohydrate-based ligand. In some embodiments, the carbohydrate-based ligand is an ASGPR ligand. In one embodiment, at least one of the targeting ligands is a GalNAc-based conjugate.

In certain embodiments, the sciRNA agent of the invention further comprises a ligand having a structure shown below:

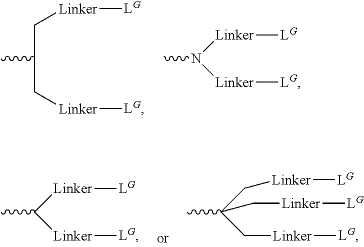

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence O or S.

In certain embodiments, the sciRNA agent of the invention comprises a ligand of Formula (II), (III), (IV) or (V):

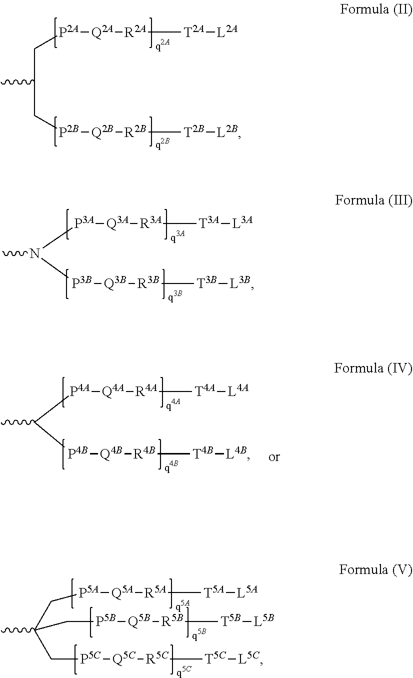

wherein:

q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;

Q and Q′ are independently for each occurrence is absent, —(P⁷-Q⁷-R⁷)ₚ-T⁷- or -T⁷-Q⁷-T^{7′}-B-T^{8′}-Q⁸-T⁸;
p^{2A}, p^{2B}, p^{3A}, p^{3B}, p^{4A}, p^{4B}, p^{5A}, p^{5B}, p^{5C}, p⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};

Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);

T^{B} and T^{B′} are each independently for each occurrence absent, CO, NH, O, S, OC(O), OC(O)O, NHC(O), NHC(O)NH, NHC(O)O, CH₂, CH₂NH or CH₂O;

R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;

R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

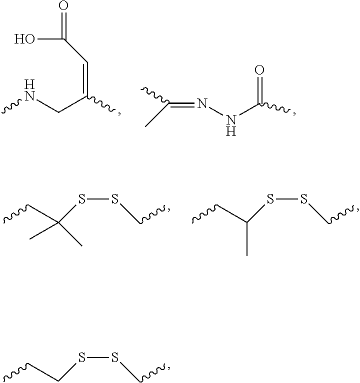

or heterocyclyl;

L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;

R′ and R″ are each independently H, C₁-C₆ alkyl, OH, SH, or N(R^{N})₂;

R^{N} is independently for each occurrence H, methyl, ethyl, propyl, isopropyl, butyl or benzyl;

R^{a} is H or amino acid side chain;

Z′, Z″, Z′″ and Z″″ are each independently for each occurrence O or S;

p represents independently for each occurrence 0-20.

As discussed above, because the ligand can be conjugated to the sciRNA agent via a linker or carrier, and because the linker or carrier can contain a branched linker, the sciRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, the ASGPR ligand conjugated to the sciRNA is one or more GalNAc derivatives attached through a bivalent or trivalent branched linker.

In certain embodiments, the sciRNA agent comprises a ligand of structure:

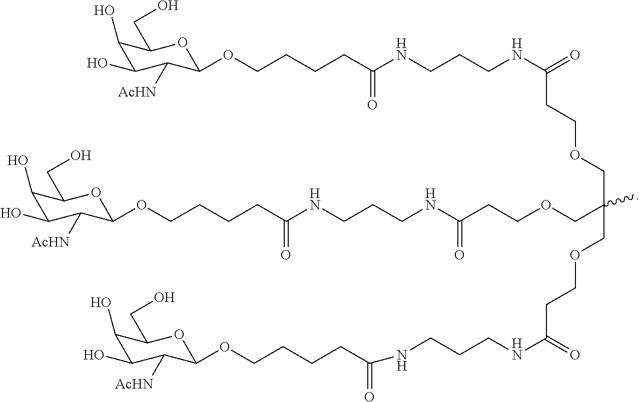 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the sciRNA agent comprises a ligand of structure:

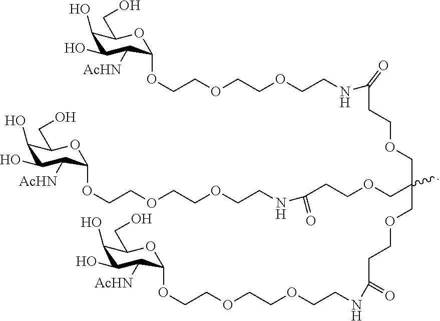

In certain embodiments, the sciRNA agent comprises a ligand of structure:

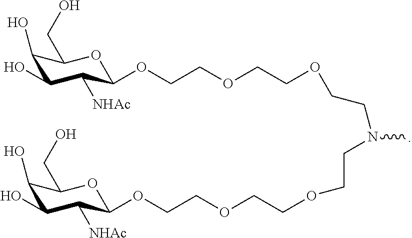

In certain embodiments, the sciRNA agent comprises a ligand of structure:

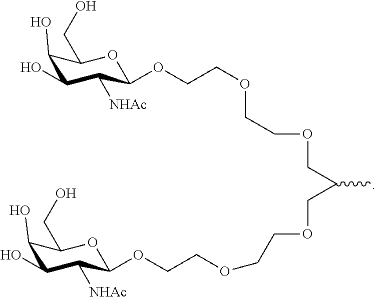

In certain embodiments, the sciRNA agent comprises a ligand of structure:

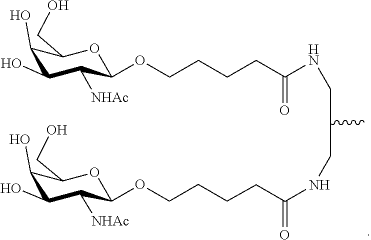

In certain embodiments, the sciRNA agent comprises a ligand of structure:

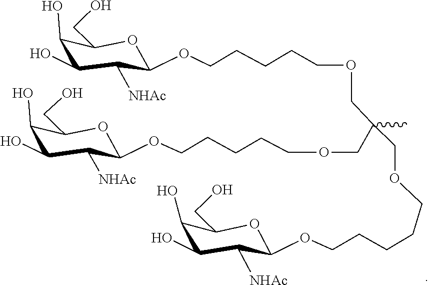

In certain embodiments, the sciRNA agent comprises a ligand of structure:

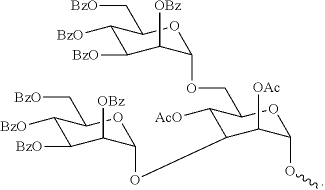

In certain embodiments, the sciRNA agent comprises a ligand of structure:

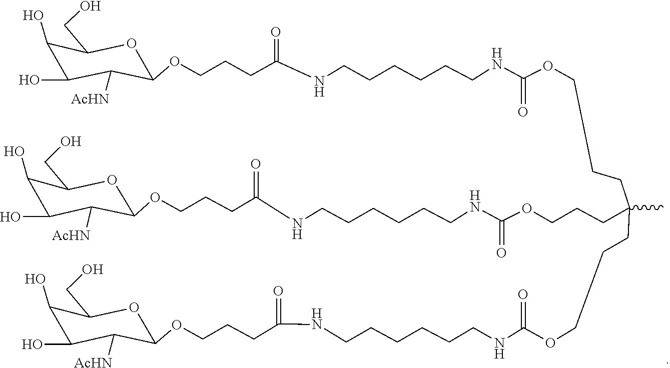 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the sciRNA agent comprises a ligand of structure:

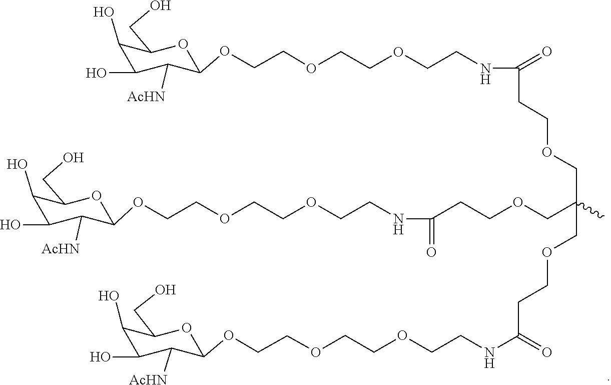 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the sciRNA agent comprises a ligand of structure:

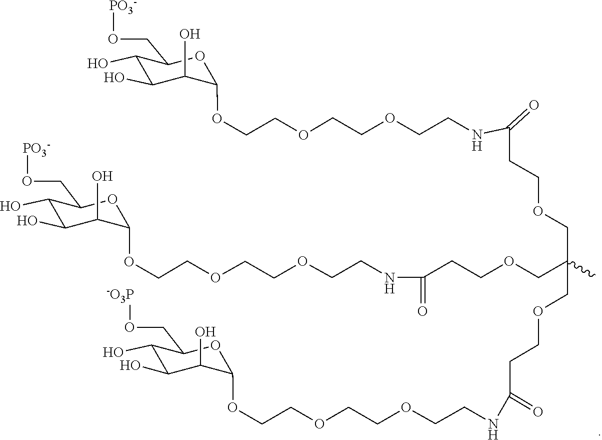

In certain embodiments, the sciRNA agent comprises a ligand of structure:

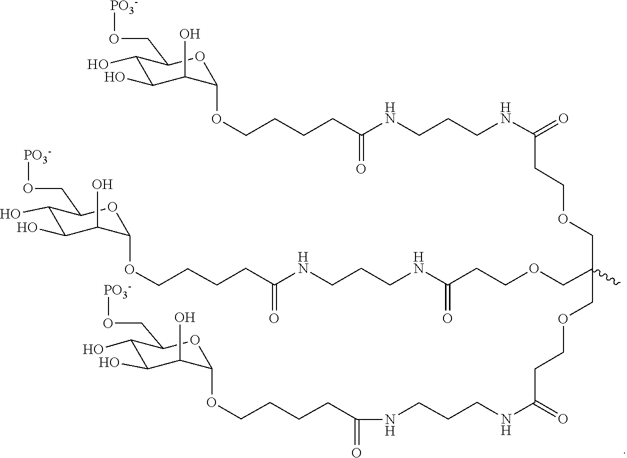

In certain embodiments, the sciRNA agent comprises a monomer of structure:

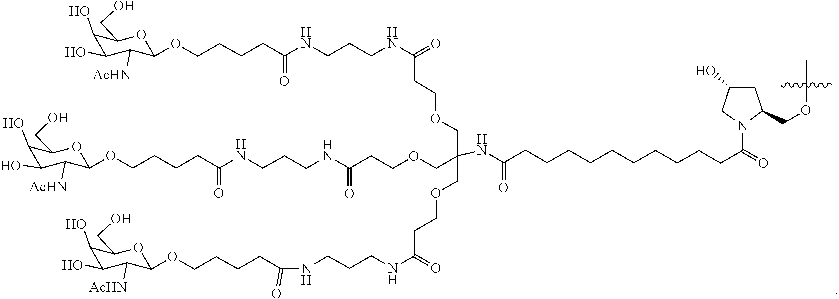 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In certain embodiments, the sciRNA agent comprises a ligand of structure:

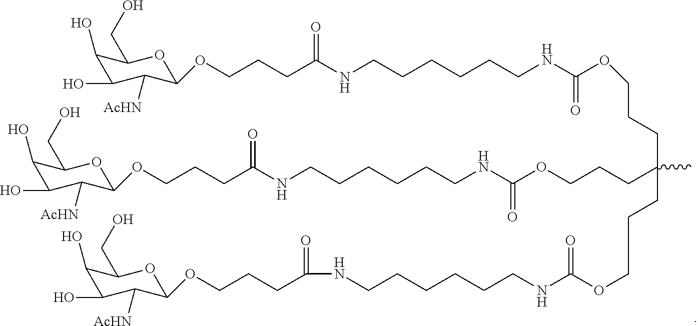 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

### Exemplary Ligand Monomers

In certain embodiments, the sciRNA agent comprises a monomer of structure:

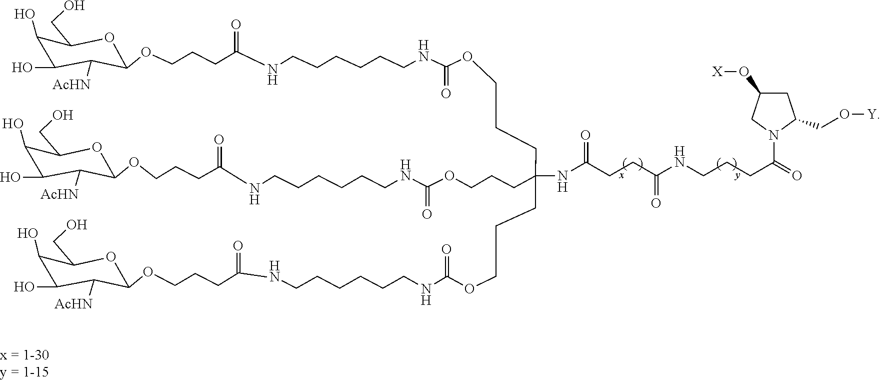

In certain embodiments, the sciRNA agent comprises a monomer of structure:

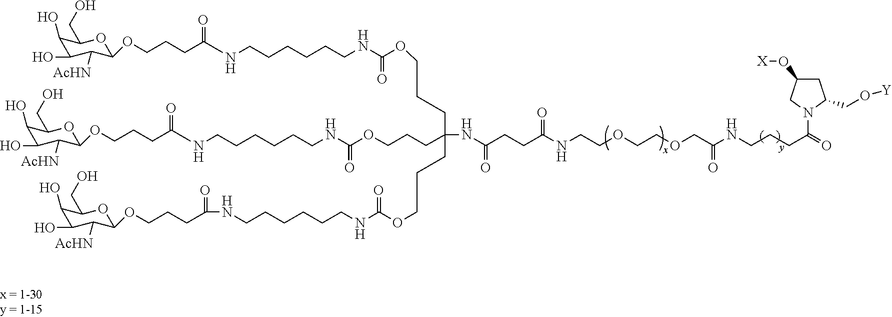

In certain embodiments, the sciRNA agent comprises a monomer of structure:

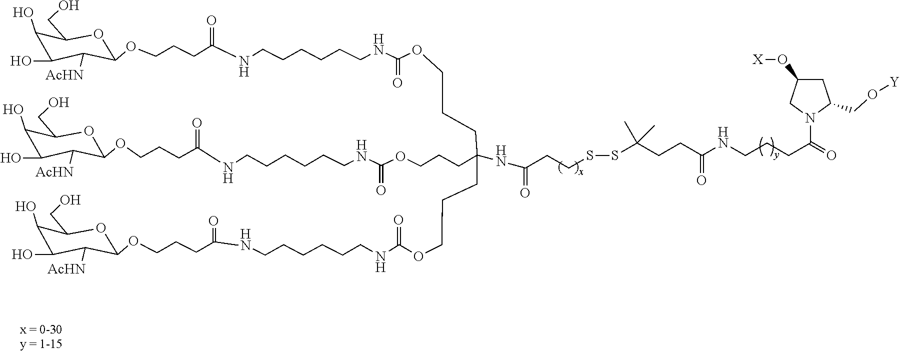

In certain embodiments, the sciRNA agent comprises a monomer of structure:

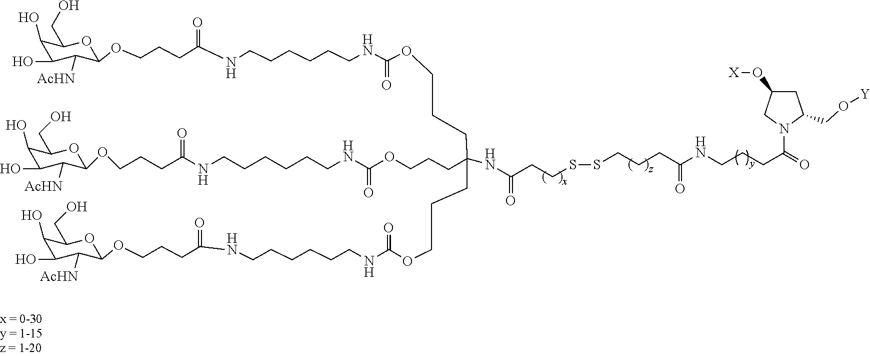

In certain embodiments, the sciRNA agent comprises a monomer of structure:

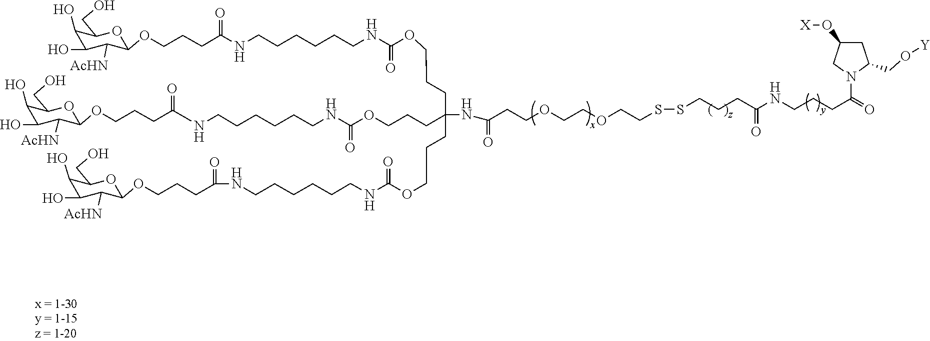

In certain embodiments, the sciRNA agent comprises a monomer of structure:

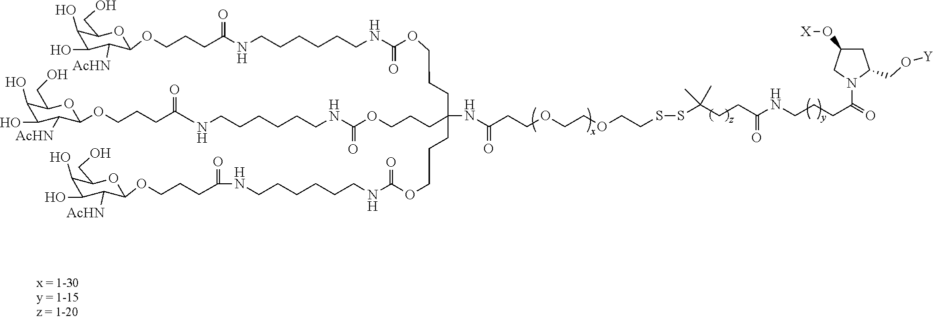

In certain embodiments, the sciRNA agent comprises a ligand of structure:

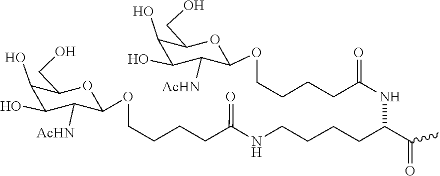

In certain embodiments, the sciRNA agent comprises a ligand of structure:

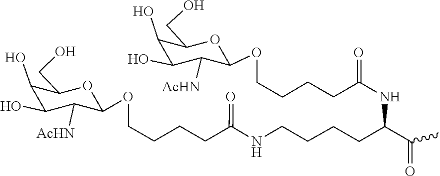

In certain embodiments, the sciRNA agent comprises a ligand of structure:

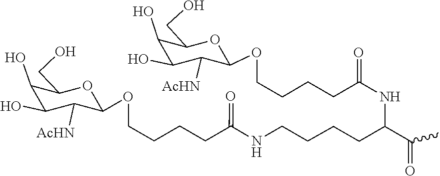

In certain embodiments, the sciRNA agent comprises a ligand of structure:

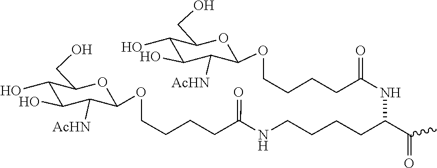

In certain embodiments, the sciRNA agent comprises a ligand of structure:

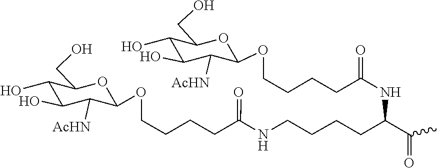

In certain embodiments, the sciRNA agent comprises a ligand of structure:

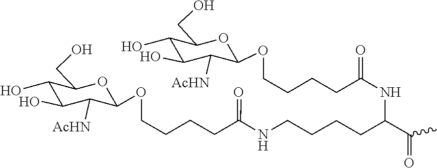

In certain embodiments, the sciRNA agent comprises a ligand of structure:

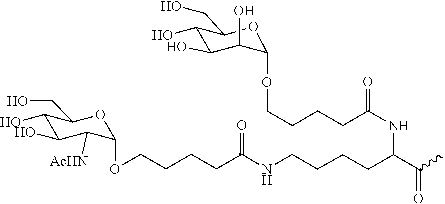

In certain embodiments, the sciRNA agent comprises a ligand of structure:

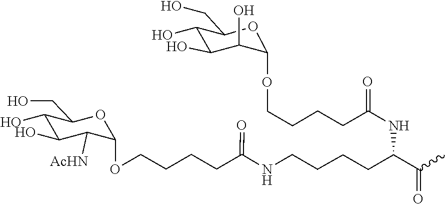

In certain embodiments, the sciRNA agent comprises a ligand of structure:

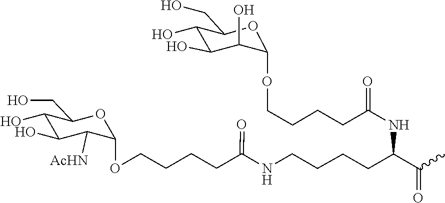

In certain embodiments, the sciRNA agent comprises a monomer of structure:

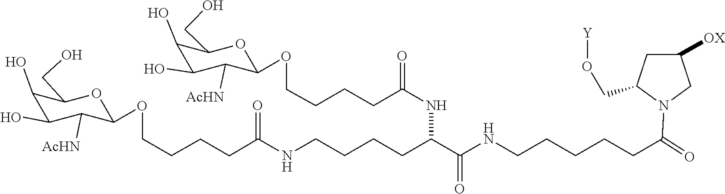

In certain embodiments, the sciRNA agent comprises a monomer of structure:

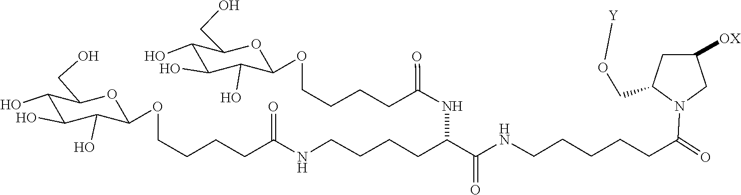

In certain embodiments, the sciRNA agent comprises a monomer of structure:

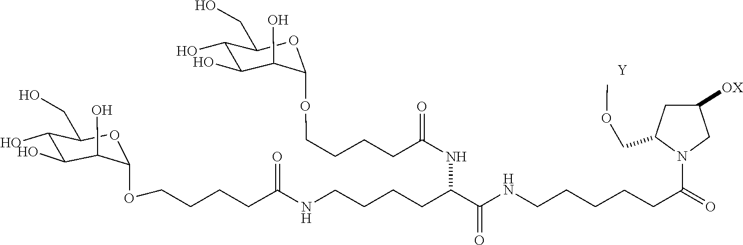

In certain embodiments, the sciRNA agent comprises a monomer of structure:

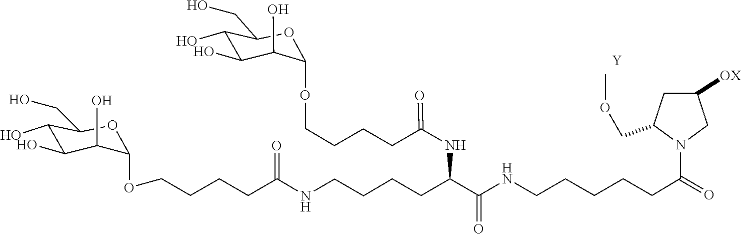

In certain embodiments, the sciRNA agent of the invention comprises a monomer of structure:

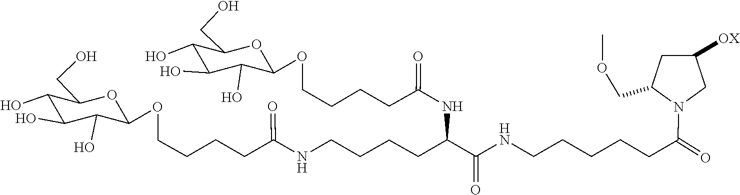

In certain embodiments, the sciRNA agent comprises a monomer of structure:

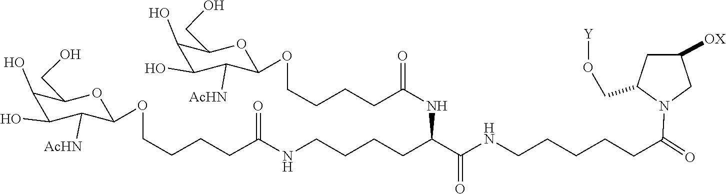

In some embodiments, both L^{2A} and L^{2B} are the same. In some embodiments, both L^{2A} and L^{2B} are different.

In some embodiments, both L^{3A} and L^{3B} are the same. In some embodiments, both L^{3A} and L^{3B} are different.

In some embodiments, both L^{4A} and L^{4B} are the same. In some embodiments, both L^{4A} and L^{4B} are different.

In some embodiments, all of L^{5A}, L^{5B} and L^{5C} are the same. In some embodiments, two of L^{5A}, L^{5B} and L^{5C} are the same. In some embodiments, L^{5A} and L^{5B} are the same. In some embodiments, L^{5A} and L^{5C} are the same. In some embodiments, L^{5B} and L^{5C} are the same.

In certain embodiments, the sciRNA agent comprises a monomer of structure:

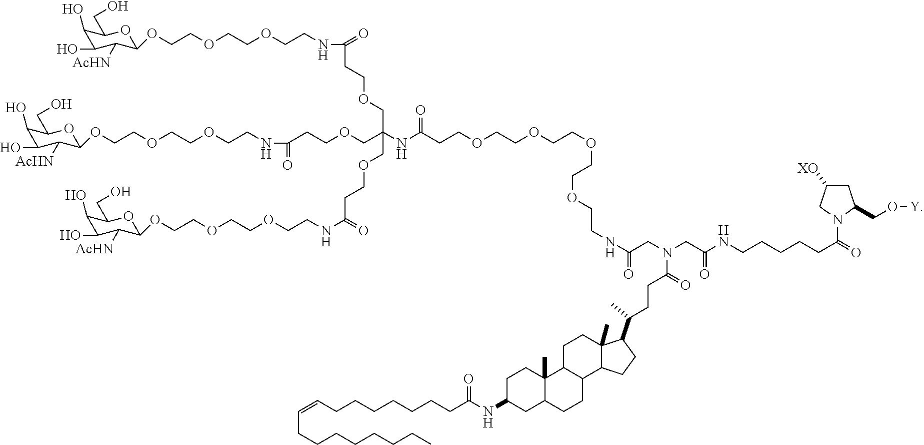

In certain embodiments, the sciRNA agent comprises a monomer of structure:

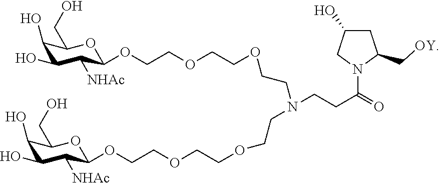

In certain embodiments, the sciRNA agent comprises a monomer of structure:

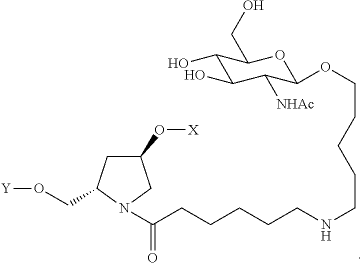

In certain embodiments, the sciRNA agent comprises a monomer of structure:

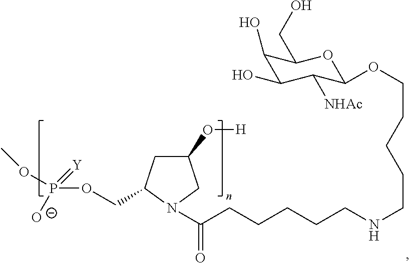

wherein Y is O or S, and n is 1-6.

In certain embodiments, the sciRNA agent comprises a monomer of structure:

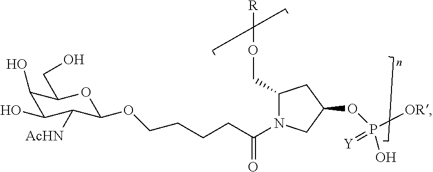

wherein Y is O or S, n is 1-6, R is hydrogen or nucleic acid, and R′ is nucleic acid.

In certain embodiments, the sciRNA agent comprises a monomer of structure:

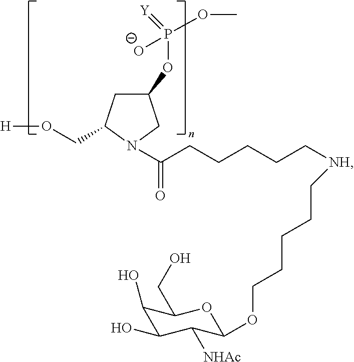

wherein Y is O or S, and n is 1-6.

In certain embodiments, the sciRNA agent comprises a monomer of structure:

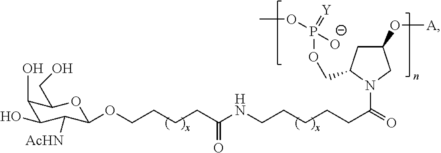

wherein Y is O or S, n is 2-6, x is 1-6, and A is H or a phosphate linkage.

In certain embodiments, the sciRNA agent comprises at least 1, 2, 3 or 4 monomer of structure:

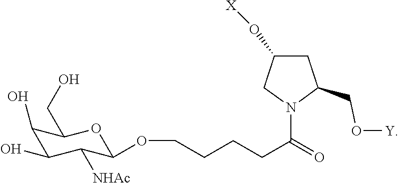

In certain embodiments, the sciRNA agent comprises a monomer of structure:

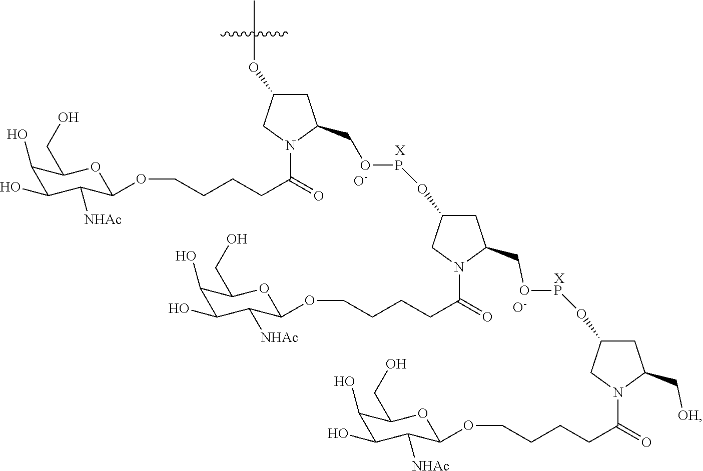

wherein X is O or S.

In certain embodiments, the sciRNA agent comprises a monomer of structure:

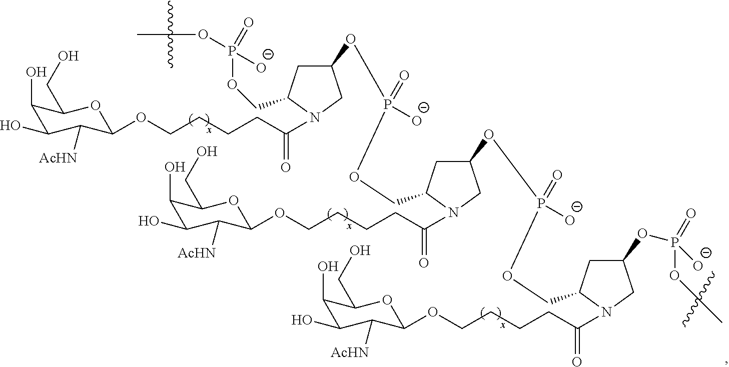

wherein x is 1-12.

In certain embodiments, the sciRNA agent comprises a monomer of structure:

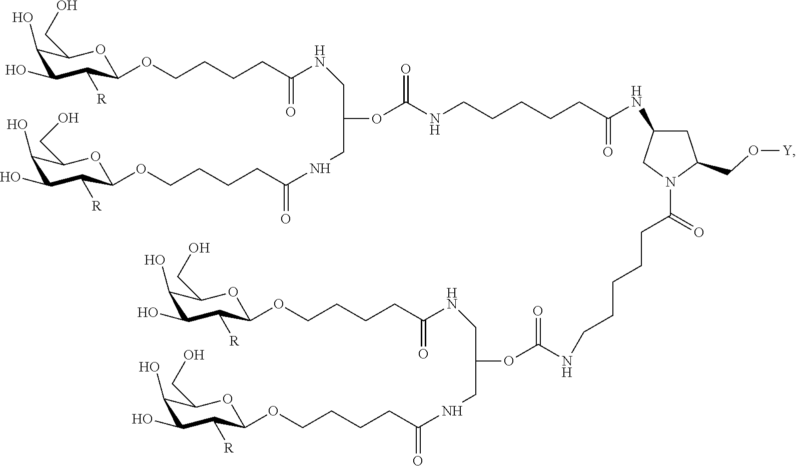

wherein R is OH or NHCOCH₃.

In certain embodiments, the sciRNA agent comprises a monomer of structure:

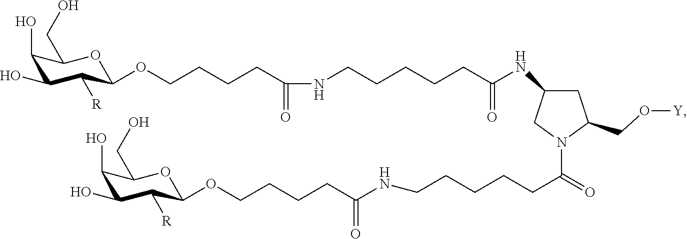

wherein R is OH or NHCOCH₃.

In certain embodiments, the sciRNA agent comprises a monomer of structure:

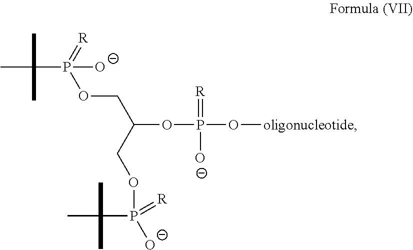

wherein R is O or S.

In certain embodiments, the sciRNA agent comprises a monomer of structure:

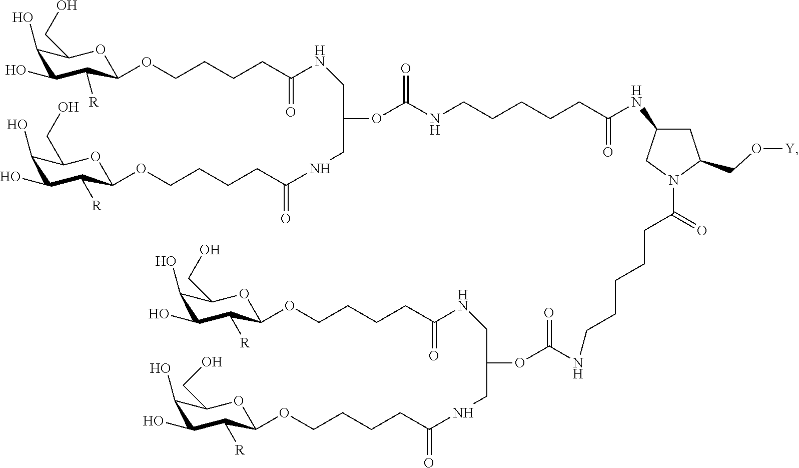

wherein R is OH or NHCOCH₃.

In certain embodiments, the sciRNA agent comprises a monomer of structure:

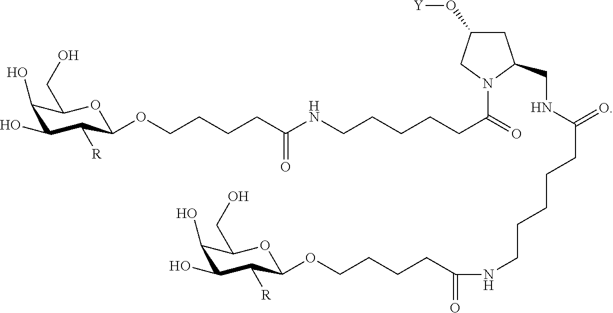

In certain embodiments, the sciRNA agent comprises a monomer of structure:

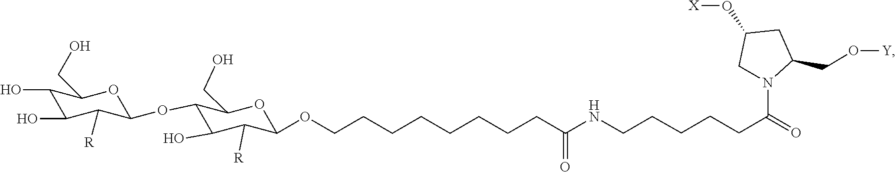

wherein R is OH or NHCOCH₃.

In certain embodiments, the sciRNA agent comprises a monomer of structure:

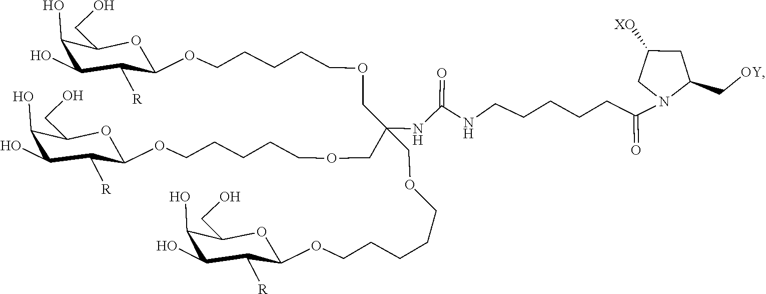

wherein R is OH or NHCOCH₃.

In certain embodiments, the sciRNA agent comprises a monomer of structure:

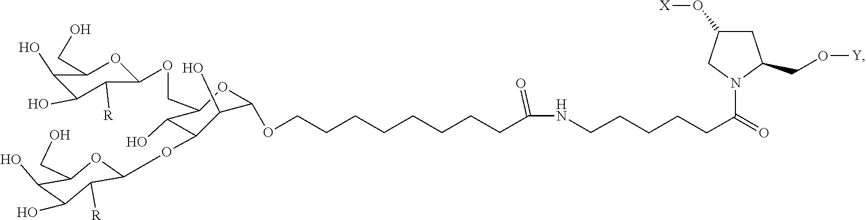

wherein R is OH or NHCOCH₃.

In certain embodiments, the sciRNA agent comprises a monomer of structure:

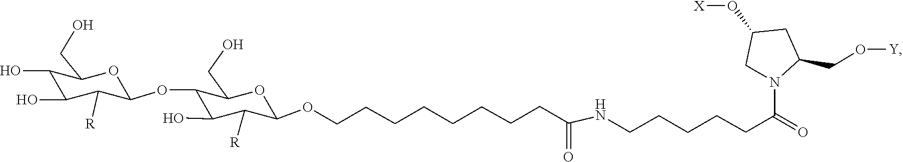

wherein R is OH or NHCOCH₃.

In certain embodiments, the sciRNA agent comprises a monomer of structure:

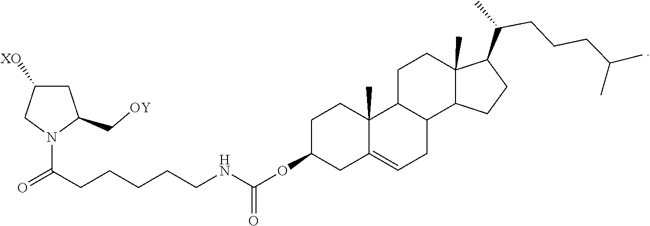

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In certain embodiments, the sciRNA agent is conjugated with a ligand of structure:

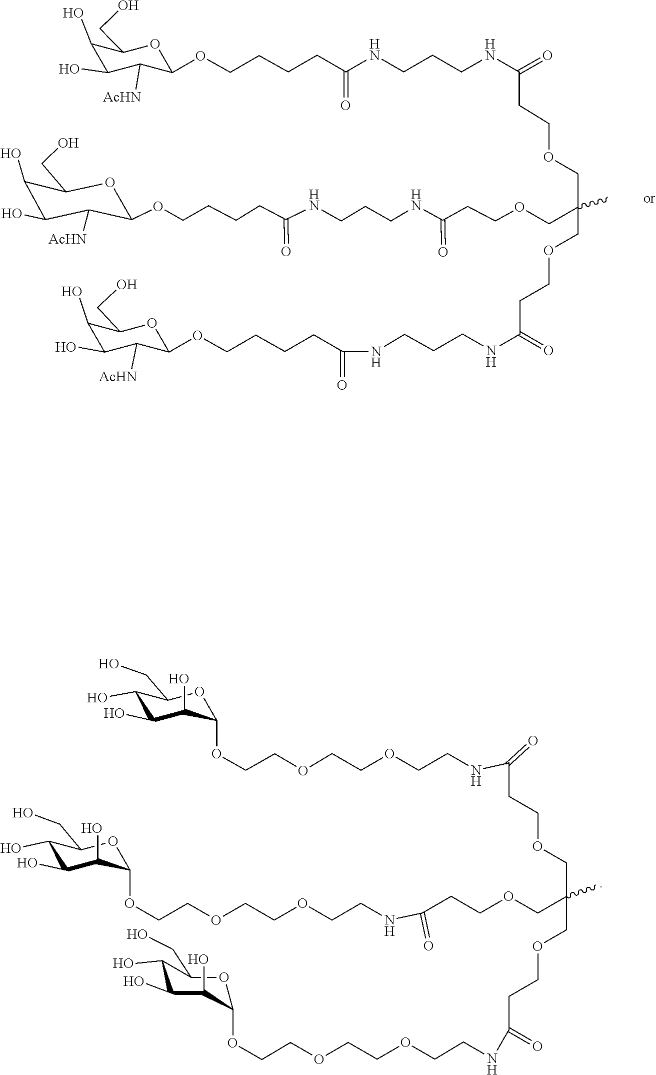

In certain embodiments, the sciRNA agent comprises a ligand of structure:

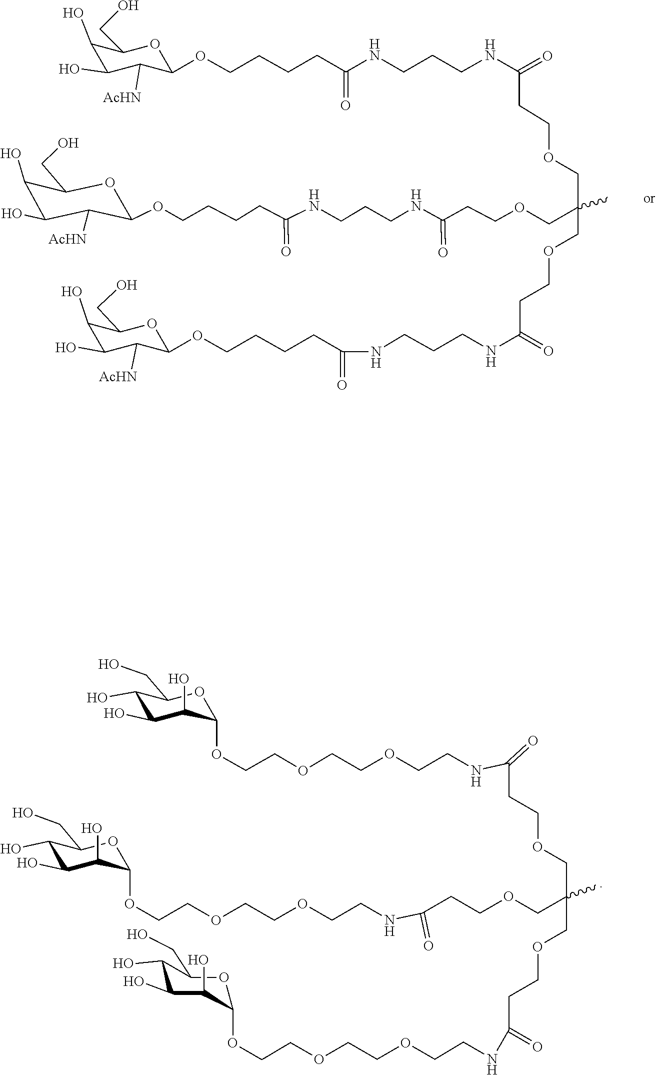

In certain embodiments, the sciRNA agent comprises a monomer of structure:

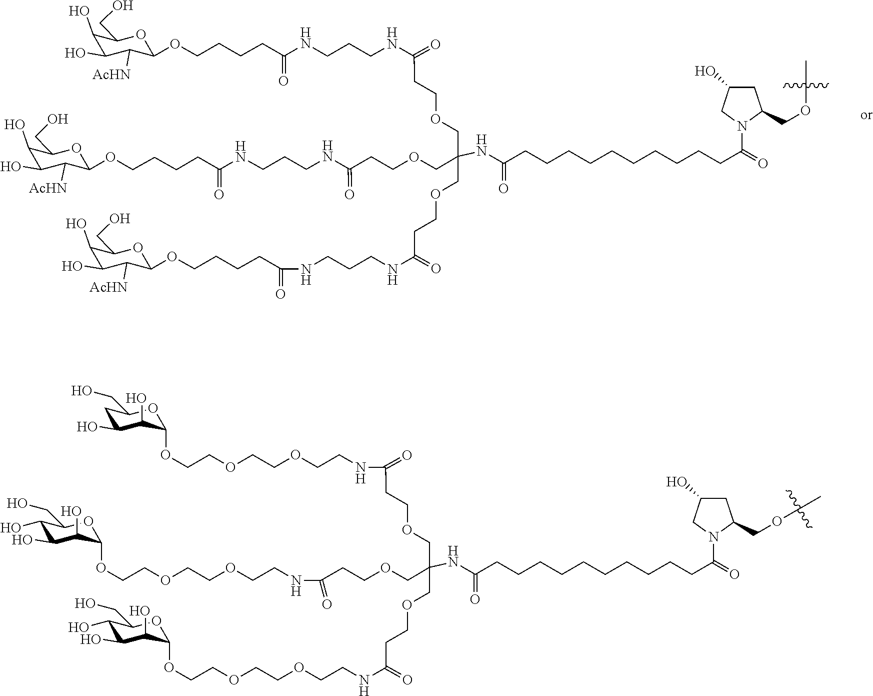

Synthesis of above described ligands and monomers is described, for example, in U.S. Pat. No. 8,106,022, content of which is incorporated herein by reference in its entirety.

In certain embodiments, at least one of the ligands conjugated to the sciRNA is a lipophilic moiety.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the sciRNA agent, conjugated to one or more lipophilic moieties, can be measured by its protein binding characteristics. For instance, the unbound fraction in the plasma protein binding assay of the sciRNA agent can be determined to positively correlate to the relative hydrophobicity of the sciRNA agent, which can positively correlate to the silencing activity of the sciRNA agent.

In one embodiment, the plasma protein binding assay determined is an electrophoretic mobility shift assay (EMSA) using human serum albumin protein. The hydrophobicity of the sciRNA agent, measured by fraction of unbound sciRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of sciRNA.

Accordingly, conjugating the lipophilic moieties to the internal position(s) of the sciRNA agent provides optimal hydrophobicity for the enhanced in vivo delivery of sciRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprises a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic moiety may be attached to the sciRNA agent by any method known in the art, including via a functional grouping already present in the lipophilic moiety or introduced into the sciRNA agent, such as a hydroxy group (e.g., —CO—CH₂—OH). The functional groups already present in the lipophilic moiety or introduced into the sciRNA agent include, but are not limited to, hydroxyl, amine, carboxylic acid, sulfonate, phosphate, thiol, azide, and alkyne.

Conjugation of the sciRNA agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the sciRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

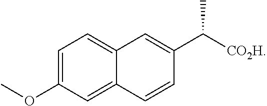 [SMILES: COc1ccc2cc([C@H](C)C(=O)O)ccc2c1]

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

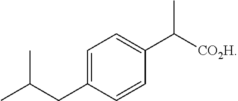

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexanoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.). In one example, the lipohilic moiety is docosahexaenoic acid.

In certain embodiments, more than one lipophilic moieties can be incorporated into the sciRNA agent, particularly when the lipophilic moiety has a low lipophilicity or hydrophobicity. In one embodiment, two or more lipophilic moieties are incorporated into the same strand of the sciRNA agent. In one embodiment, each strand of the sciRNA agent has one or more lipophilic moieties incorporated. In one embodiment, two or more lipophilic moieties are incorporated into the same position (i.e., the same nucleobase, same sugar moiety, or same internucleosidic linkage) of the sciRNA agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the sciRNA agent via a direct attachment to the ribosugar of the sciRNA agent. Alternatively, the lipophilic moiety may be conjugated to the sciRNA agent via a linker or a carrier.

In certain embodiments, the lipophilic moiety may be conjugated to the sciRNA agent via one or more linkers (tethers).

In one embodiment, the lipophilic moiety is conjugated to the sciRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the over all double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

The term “sciRNA” as used herein, refers to a small circular iRNA agent, that has at least one strand (e.g, a sense strand) that has a circular or substantially circular structure, whereas the other strand (e.g., an antisense strand) can have a linear structure that is annealed to the strand that has a circular or substantially circular structure. Alternatively, the sciRNA can have a circular or substantially circular antisense strand and a linear sense strand that is annealed to the circular or substantially circular antisense strand. It is also possible both sense strand and antisense strands have a circular or substantially circular structure.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA. The % and/or fold difference can be calculated relative to the control or the non-control, for example,

$$\% difference = \frac{\begin{matrix}[ expression with siRNA - \\ expression without siRNA ]\end{matrix}}{expression without siRNA}$$

$$or$$

$$\% difference = \frac{\begin{matrix}[ expression with siRNA - \\ expression without siRNA ]\end{matrix}}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The sciRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the sciRNA comprises a sense strand and an antisense strand, wherein the antisense strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, /. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a sciRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a sciRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a sciRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the sciRNA agent are each 15 to 30 nucleotides in length.

In one embodiment, the sense and antisense strands of the sciRNA agent are each 19 to 25 nucleotides in length.

In one embodiment, the sense and antisense strands of the sciRNA agent are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the sciRNA agent comprises a single-stranded overhang on at least one of the termini. In one embodiment, the single-stranded overhang is 1, 2, or 3 nucleotides in length.

In one embodiment, the sense strand of the sciRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the sciRNA has a ZXY structure, such as is described in PCT Publication No. 2004080406, which is hereby incorporated by reference in its entirety.

In certain embodiment, the two nucleotide sequences can be linked together to form a long strand. The two nucleotide sequences can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two nucleotide sequences can also be linked together by a non-nucleotide based linker, e.g. a linker described herein. It will be appreciated by one of skill in the art that any oligonucleotide chemical modifications or variations describe herein can be used in the oligonucleotide linker.

In certain embodiments, two strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

sciRNA Sequences Design

In one embodiment, the sciRNA agent of the invention comprises a double ended bluntmer of 19 nt in length, wherein the sense strand contains at least one motif of three 2′-F modifications on three consecutive nucleotides at positions 7,8,9 from the 5′end. The antisense strand contains at least one motif of three 2′-O-methyl modifications on three consecutive nucleotides at positions 11,12,13 from the 5′end.

In one embodiment, the sciRNA agent of the invention comprises a double ended bluntmer of 20 nt in length, wherein the sense strand contains at least one motif of three 2′-F modifications on three consecutive nucleotides at positions 8,9,10 from the 5′ end. The antisense strand contains at least one motif of three 2′-O-methyl modifications on three consecutive nucleotides at positions 11,12,13 from the 5′end.

In one embodiment, the sciRNA agent of the invention comprises a double ended bluntmer of 21 nt in length, wherein the sense strand contains at least one motif of three 2′-F modifications on three consecutive nucleotides at positions 9,10,11 from the 5′end. The antisense strand contains at least one motif of three 2′-O-methyl modifications on three consecutive nucleotides at positions 11,12,13 from the 5′end.

In one embodiment, the sciRNA agent of the invention comprises a 21 nucleotides (nt) sense strand and a 23 nucleotides (nt) antisense strand, wherein the sense strand contains at least one motif of three 2′-F modifications on three consecutive nucleotides at positions 9,10,11 from the 5′ end; the antisense strand contains at least one motif of three 2′-O-methyl modifications on three consecutive nucleotides at positions 11,12,13 from the 5′end, wherein one end of the sciRNA is blunt, while the other end is comprises a 2 nt overhang. Preferably, the 2 nt overhang is at the 3′-end of the antisense strand. Optionally, the sciRNA agent further comprises a ligand (e.g., GalNAc₃).

In one embodiment, the sciRNA agent of the invention comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3 ‘ terminal nucleotide of antisense strand is unpaired with sense strand, and up to 6 consecutive 3’ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the sciRNA agent of the invention comprises a sense and antisense strands, wherein said sciRNA agent comprises a first strand having a length which is at least 25 and at most 29 nucleotides and a second strand having a length which is at most 30 nucleotides with at least one motif of three 2′-O-methyl modifications on three consecutive nucleotides at position 11,12,13 from the 5′ end; wherein said 3′ end of said first strand and said 5′ end of said second strand form a blunt end and said second strand is 1˜4 nucleotides longer at its 3′ end than the first strand, wherein the duplex region region which is at least 25 nucleotides in length, and said second strand is sufficiently complementary to a target mRNA along at least 19 nt of said second strand length to reduce target gene expression when said sciRNA agent is introduced into a mammalian cell, and wherein dicer cleavage of said sciRNA preferentially results in an siRNA comprising said 3′ end of said second strand, thereby reducing expression of the target gene in the mammal. Optionally, the sciRNA agent further comprises a ligand (e.g., GalNAc₃).

In one embodiment, the sense strand of the sciRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand of the sciRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For sciRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the sciRNA from the 5′-end.

In some embodiments, the sciRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the anti sense strand.

In some embodiments, the sciRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the sciRNA agent comprises a sense strand and antisense strand each having 14 to 30 nucleotides, wherein the sense strand contains at least one motif of three 2′-F modifications on three consecutive nucleotides at positions 9,10,11 from the 5′end, and wherein the antisense strand contains at least one motif of three 2′-O-methyl modifications on three consecutive nucleotides at positions 11,12,13 from the 5′end.

In one embodiment, the sciRNA agent of the invention comprises mismatch(es) with the target, within the duplex, or combinations thereof. The mistmatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the sciRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In some embodiments, the invention relates to an sciRNA agent for inhibiting the expression of a target gene. The sciRNA agent comprises a sense strand and an antisense strand, each strand having 14 to 45 nucleotides. The sciRNA agent is represented by formula (I):

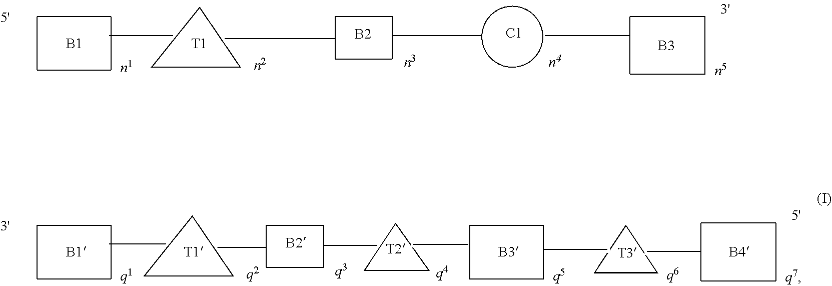

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

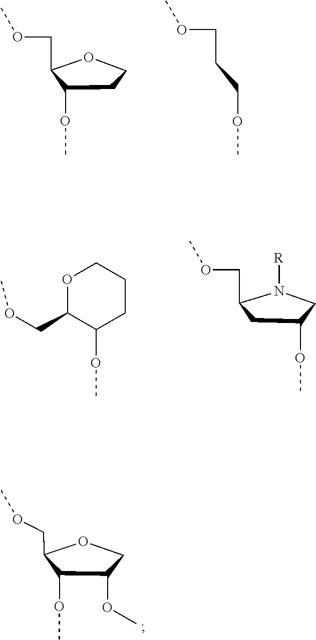

and iii) sugar modification selected from the group consisting of:

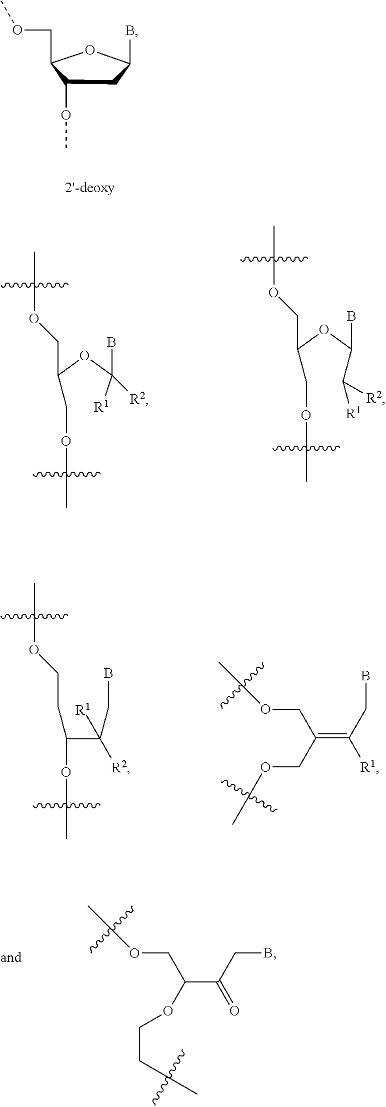

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

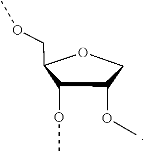

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1, T1′, T2′, and T3′ are each independently 2′-F. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0. q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

Additional exemplary embodiments for the sequence designs of the sciRNA agent represented by formula (I) can be found in WO 2016/028649, which is incorporated herein by reference in its entirety.

The sciRNA agent can comprise a phosphorus-containing group at the 5′-end of of a sense or antisense nucleotide sequence. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

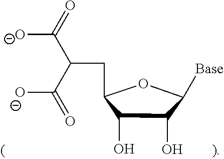

When the 5′-end phosphorus-containing group is 5′-end vinylphosphonate (5′-VP), the 5′-VP can be either 5′-E-VP isomer (i.e., trans-vinylphosphate,

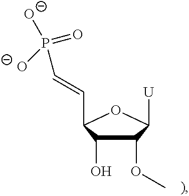

5′-Z-VP isomer (i.e., cis-vinylphosphate,

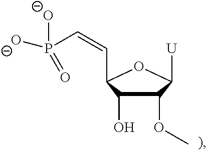

or mixtures thereof.

In one embodiment, the sciRNA agent comprises a phosphorus-containing group at the 5′-end of a sense nucleotide sequence. In one embodiment, the sciRNA agent comprises a phosphorus-containing group at the 5′-end of an antisense nucleotide sequence.

In one embodiment, the sciRNA agent comprises a 5′-P. In one embodiment, the sciRNA agent comprises a 5′-P in an antisense nucleotide sequence.

In one embodiment, the sciRNA agent comprises a 5′-PS. In one embodiment, the sciRNA agent comprises a 5′-PS in an antisense nucleotide sequence.

In one embodiment, the sciRNA agent comprises a 5′-VP. In one embodiment, the sciRNA agent comprises a 5′-VP in an antisense nucleotide sequence. In one embodiment, the sciRNA agent comprises a 5′-E-VP in an antisense nucleotide sequence. In one embodiment, the sciRNA agent comprises a 5′-Z-VP in an antisense nucleotide sequence.

In one embodiment, the sciRNA agent comprises a 5′-PS₂. In one embodiment, the sciRNA agent comprises a 5′-PS₂ in an antisense nucleotide sequence.

In one embodiment, the sciRNA agent comprises a 5′-PS₂. In one embodiment, the sciRNA agent comprises a 5′-deoxy-5′-C-malonyl in an antisense nucleotide sequence.

In one embodiment, 100%, 95%, 90%, 85%, 80%, 75%, 70%, 65%, 60%, 55%, 50%, 45%, 40%, 35% or 30% of the sciRNA agent of the invention is modified. For example, when 50% of the sciRNA agent is modified, 50% of all nucleotides present in the sciRNA agent contain a modification as described herein.

In one embodiment, each of the sense and antisense strands of the sciRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the sciRNA agent contains at least two different modifications.

In one embodiment, the sciRNA agent of Formula (I) further comprises 3′ and/or 5′ overhang(s) of 1-10 nucleotides in length. In one example, sciRNA agent of formula (I) comprises a 3′ overhang at the 3′-end of the antisense strand and a blunt end at the 5′-end of the antisense strand. In another example, the sciRNA agent has a 5′ overhang at the 5′-end of the sense strand.

In one embodiment, the sciRNA agent of the invention does not contain any 2′-F modification.

In one embodiment, the sense strand and/or antisense strand of the sciRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the sciRNA agent of the invention is 100% complementary to a target RNA to hybridize thereto and inhibits its expression through RNA interference. In another embodiment, the antisense strand of the sciRNA agent of the invention is at least 95%, at least 90%, at least 85%, at least 80%, at least 75%, at least 70%, at least 65%, at least 60%, at least 55%, or at least 50% complementary to a target RNA.

In some embodiments, the invention relates to a sciRNA agent as defined herein capable of inhibiting the expression of a target gene. The sciRNA agent comprises a sense strand and an antisense strand, each strand having 14 to 45 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the sciRNA represented by formula (I) can also apply to the sciRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′ end of the antisense strand.

For example, the sciRNA agent as defined herein can comprise i) a phosphorus-containing group at the 5′-end of the sense strand or antisense strand; ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) a ligand, such as a ASGPR ligand (e.g., one or more GalNAc derivatives) at 5′-end or 3′-end of the sense strand or antisense strand. For instance, the ligand may be at the 3′-end of the sense strand.

In some embodiments, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker; and
(iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
   and
   (b) an antisense strand having:

(i) a length of 23 nucleotides;
(ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
(iii) phosphorothioate internucleotide linkages between nucleotide positions 21 and 22, and between nucleotide positions 22 and 23 (counting from the 5′ end);
wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, 10, 12, 14, and 16 to 21, and 2′-F modifications at positions 7, 9, 11, 13, and 15; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 9, and 12 to 21, and 2′-F modifications at positions 10, and 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-F modifications at positions 1, 3, 5, 7, 9 to 11, and 13, and 2′-OMe modifications at positions 2, 4, 6, 8, 12, and 14 to 21; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
(iv) phosphorothioate internucleotide linkages between nucleotide positions 1 and 2, and between nucleotide positions 2 and 3 (counting from the 5′ end);
and
   (b) an antisense strand having:
(i) a length of 25 nucleotides;
(ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a four nucleotide overhang at the 3′-end of the antisense strand, and a blunt end at the 5′-end of the antisense strand.

In another embodiment, the sciRNA agents of the present invention comprise:
(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 21 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the sciRNA agents have a two nucleotide overhang at the 3′-end of the antisense strand, and a blunt end at the 5′-end of the antisense strand.

In another embodiment, the sciRNA agents of the present invention comprise:

(a) a sense strand having:
(i) a length of 19 nucleotides;
(ii) optionally an ASGPR ligand attached to the 3′-end, wherein said ASGPR ligand comprises three GalNAc derivatives attached through a trivalent branched linker;
(iii) 2′-OMe modifications at positions 1 to 4, 6, and 10 to 19, and 2′-F modifications at positions 5, and 7 to 9; and
(iv) phosphorothioate internucleotide linkages between nucleotide positions 1 and 2, and between nucleotide positions 2 and 3 (counting from the 5′ end);
and
   (b) an antisense strand having:
(i) a length of 21 nucleotides;
(ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
   wherein the sciRNA agents have a two nucleotide overhang at the 3′-end of the antisense strand, and a blunt end at the 5′-end of the antisense strand.

In one embodiment, the sciRNA agents of the present invention comprise:

(a) a sense strand having:
(i) a length of 18-23 nucleotides;
(ii) three consecutive 2′-F modifications at positions 7-15; and
   (b) an antisense strand having:
(i) a length of 18-23 nucleotides;
(ii) at least 2′-F modifications anywhere on the strand; and
(iii) at least two phosphorothioate internucleotide linkages at the first five nucleotides (counting from the 5′ end);
   wherein the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the sciRNA agents of the present invention comprise:

(a) a sense strand having:
(i) a length of 18-23 nucleotides;
(ii) less than four 2′-F modifications;
   (b) an antisense strand having:
(i) a length of 18-23 nucleotides;
(ii) at less than twelve 2′-F modification; and
(iii) at least two phosphorothioate internucleotide linkages at the first five nucleotides (counting from the 5′ end);
   wherein the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the sciRNA agents of the present invention comprise:

(a) a sense strand having:
(i) a length of 19-35 nucleotides;
(ii) less than four 2′-F modifications;
   (b) an antisense strand having:
(i) a length of 19-35 nucleotides;
(ii) at less than twelve 2′-F modification; and
(iii) at least two phosphorothioate internucleotide linkages at the first five nucleotides (counting from the 5′ end);
   wherein the duplex region is between 19 to 25 base pairs (preferably 19, 20, 21 or 22); and wherein the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In some embodiments, the sciRNA contains a sense strand sequence that can be represented by formula (II):

5′nₚ-Nₐ—(XXX)ᵢ—N_{b}—YYY—N_{b}—(ZZZ)ⱼ—Nₐ-n_{q}3′ (II)

wherein:

i and j are each independently 0 or 1;

p and q are each independently 0-6;

each Nₐ independently represents an oligonucleotide sequence comprising 0-25 modified nucleotides, each sequence comprising at least two differently modified nucleotides;

each N_{b} independently represents an oligonucleotide sequence comprising 1, 2, 3, 4, 5, or 6 modified nucleotides;

each nₚ and n_{q} independently represent an overhang nucleotide;

wherein N_{b} and Y do not have the same modification;

wherein XXX, YYY and ZZZ each independently represent one motif of three identical modifications on three consecutive nucleotides;

wherein wherein the sciRNA agents have one or more lipophilic moieties conjugated to one or more positions on at least one strand; and

wherein the antisense strand of the sciRNA comprises two blocks of one, two pr three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the sciRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, the sciRNA agent contains one, two, three, four, five, six, seven, eight, nine, ten, eleven or twelve 2′-F modification(s). In one example, sciRNA agent contains nine or ten 2′-F modifications.

The sciRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the sciRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

In some embodiments, the compound of the invention disclosed herein is an antimir. In some embodiments, compound of the invention comprises at least two antimirs covalently linked to each other via a nucleotide-based or non-nucleotide-based linker, for example a linker described in the disclosure, or non-covalently linked to each other. The terms “antimir” “microRNA inhibitor” or “miR inhibitor” are synonymous and refer to oligonucleotides or modified oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

In some embodiments, compound of the invention disclosed herein is an antagomir. In some embodiments, the compound of the invention comprises at least two antagomirs covalently linked to each other via a nucleotide-based or non-nucleotide-based linker, for example a linker described in the disclosure, or non-covalently linked to each other. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In a preferred embodiment, antagomir comprises a 2′-O-methyl modification at all nucleotides, a cholesterol moiety at 3′-end, two phsophorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Recent studies have found that dsRNA can also activate gene expression, a mechanism that has been termed “small RNA-induced gene activation” or RNAa (activating RNA). See for example Li, L. C. et al. Proc Natl Acad Sci USA. (2006), 103(46):17337-42 and Li L. C. (2008). “Small RNA-Mediated Gene Activation”. RNA and the Regulation of Gene Expression: A Hidden Layer of Complexity. Caister Academic Press. ISBN 978-1-904455-25-7. It has been shown that dsRNAs targeting gene promoters induce potent transcriptional activation of associated genes. Endogenous miRNA that cause RNAa has also been found in humans. Check E. Nature (2007). 448 (7156): 855-858.

Another surprising observation is that gene activation by RNAa is long-lasting. Induction of gene expression has been seen to last for over ten days. The prolonged effect of RNAa could be attributed to epigenetic changes at dsRNA target sites. In some embodiments, the RNA activator can increase the expression of a gene. In some embodiments, increased gene expression inhibits viability, growth development, and/or reproduction.

Accordingly, in some embodiments, compound of the invention disclosed herein is activating RNA. In some embodiments, the compound of the invention comprises at least two activating RNAs covalently linked to each other via a nucleotide-based or non-nucleotide-based linker, for example a linker described in the disclosure, or non-covalently linked to each other.

Accordingly, in some embodiments, compound of the invention disclosed herein is a triplex forming oligonucotide (TFO). In some embodiments, the compound of the invention comprises at least two TFOs covalently linked to each other via a nucleotide-based or non-nucleotide-based linker, for example a linker described in the disclosure, or non-covalently linked to each other. Recent studies have shown that triplex forming oligonucleotides can be designed which can recognize and bind to polypurine/polypyrimidine regions in double-stranded helical DNA in a sequence-specific manner. These recognition rules are outline by Maher III, L. J., et al., Science (1989) vol. 245, pp 725-730; Moser, H. E., et al., Science (1987) vol. 238, pp 645-630; Beal, P. A., et al., Science (1992) vol. 251, pp 1360-1363; Conney, M., et al., Science (1988) vol. 241, pp 456-459 and Hogan, M. E., et al., EP Publication 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 1 12:487-94). In general, the triplex-forming oligonucleotide has the sequence correspondence:

oligo 3′-A G G T
duplex 5′-A G C T
duplex 3′-T C G A

However, it has been shown that the A-AT and G-GC triplets have the greatest triple helical stability (Reither and Jeltsch, BMC Biochem, 2002, Seρtl2, Epub). The same authors have demonstrated that TFOs designed according to the A-AT and G-GC rule do not form non-specific triplexes, indicating that the triplex formation is indeed sequence specific.

Thus for any given sequence a triplex forming sequence can be devised. Triplex-forming oligonucleotides preferably are at least 15, more preferably 25, still more preferably 30 or more nucleotides in length, up to 50 or 100 nucleotides.

Formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFGl and endogenous HPRT genes in mammalian cells (Vasquez et al., Nucl Acids Res. 1999; 27: 1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-I gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Pat. App. Nos. 2003 017068 and 2003 0096980 to Froehler et al, and 2002 0128218 and 2002 0123476 to Emanuele et al, and U.S. Pat. No. 5,721,138 to Lawn, contents of which are herein incorporated in their entireties.

### Nucleic Acid Modifications

In some embodiments, the sciRNA agent of the invention comprises at least one nucleic acid modification described herein. For example, at least one modification selected from the group consisting of modified internucleoside linkage, modified nucleobase, modified sugar, and any combinations thereof. Without limitations, such a modification can be present anywhere in the sciRNA agent of the invention. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouraci1,4-(thio)pseudouraci1,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho--(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### Nucleic Acid Modifications (Sugar)

Double-stranded sciRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ-, —[C(R1)(R2)]ₙ—O—, —[C(R1)(R2)]ₙ—N(R1)-, —[C(R1)(R2)]ₙ—N(R1)-O—, —[C(R1R2)]ₙ—O—N(R1)-, —C(R1)=C(R2)-O—, —C(R1)=N—, —C(R1)=N—O—, —C(═NR1)-, —C(═NR1)-O—, C(═O)—, —C(═O)O—, —C(═S)—, —C(═S)O—, —C(═S)S—, —O—, —Si(R1)2-, —S(═O)ₓ— and —N(R1)-;

wherein:

x is 0, 1, or 2;

n is 1, 2, 3, or 4;

each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and

each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′-wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R=H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The sciRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. The sciRNA agent of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in α configuration.

Sugar modifications can also include acyclic nucleotides, wherein a C—C bonds between ribose carbons (e.g., C1′-C2′, C2′-C3′, C3′-C4′, C4′-C4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

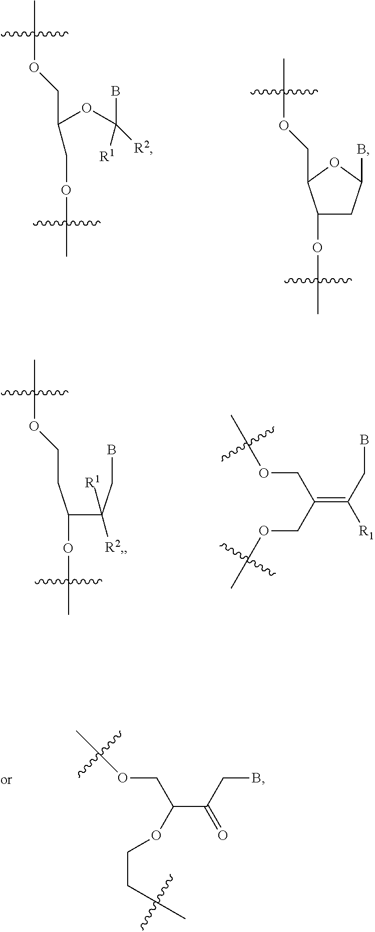

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O-CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-0-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

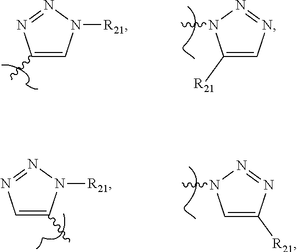

NR₂₁R₃₁, CONR₂₁R₃₁, CON(H)NR₂₁R₃₁, ONR₂₁R₃₁, CON(H)N═CR⁴¹R₅₁, N(R²¹)C(═NR₃₁)NR₂₁R₃₁, N(R₂₁)C(O)NR₂₁R₃₁, N(R₂₁)C(S)NR²¹R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R_{11′}; and R₁₁ and R_{11′} are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

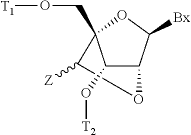

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain embodiments, the compounds of the invention comprise at least one monomer of the formula:

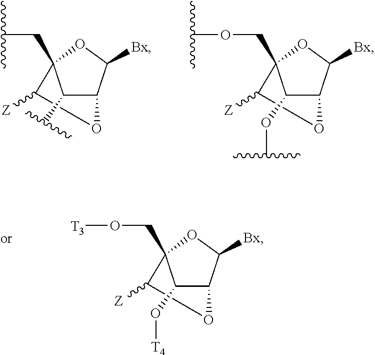

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below:

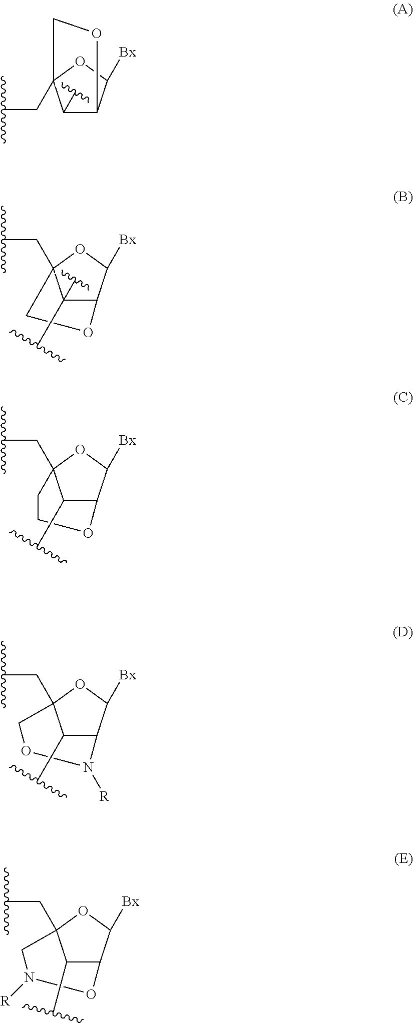

In certain embodiments, the sciRNA agent comprises at least two regions of at least two contiguous monomers of the above formula. In certain embodiments, the sciRNA agent comprises a gapped motif. In certain embodiments, the sciRNA agent comprises at least one region of from about 8 to about 14 contiguous β-D-2′-deoxyribofuranosyl nucleosides. In certain embodiments, the sciRNA agent of the invention comprises at least one region of from about 9 to about 12 contiguous β-D-2′-deoxyribofuranosyl nucleosides.

In certain embodiments, the sciRNA agent comprises at least one (e.g., 1, 2, 3, 4, 5, 6, 7, 8, 9, 10, 11, 12, 13, 14, 15 or more) comprises at least one (S)-cEt monomer of the formula:

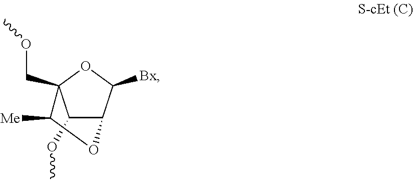

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR² (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′—O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′-N(H)—C(═O)—CH₂—S-C5′, C3′-O—P(O)—O—SS-C5′, C3′-CH₂—NH—NH-C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the sciRNA agent comprises at least one (e.g., 1, 2, 3, 4, 5, 6, 7, 8, 9, 10, 11, 12, 13, 14, 15 or more and upto including all) modified or nonphosphodiester linkages. In some embodiments, the sciRNA agent comprises at least one (e.g., 1, 2, 3, 4, 5, 6, 7, 8, 9, 10, 11, 12, 13, 14, 15 or more and upto including all) phosphorothioate linkages.

The sciRNA agent can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). It can be desirable, in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The sciRNA agent described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the sciRNA agent of the inventions provided herein are all such possible isomers, as well as their racemic and optically pure forms.

### Nucleic Acid Modifications (Terminal Modifications)

Ends of a sense or antisense nucleotide sequence of the sciRNA agent can be modified. Such modifications can be at one end or both ends of the nucleotide sequence. For example, the 3′ and/or 5′ ends can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end of a sequence with phosphate or phosphate analogs. In certain embodiments, the 5′ end of sequence is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

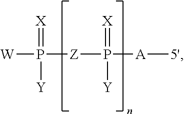

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5); 5′-triphosphate ((HO)₂(O)P—O—(HO)(O)P—O—P(HO)(O)—O-5); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5% (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[(CH₂)ₐ—P(X)(OH)—O]_{b}-5′, ((HO)2(X)P—[—(CH₂)ₐ—O—P(X)(OH)—O]_{b}-5; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as iRNAs or sciRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nuceltic acid (GNA).

Exemplified abasic modifications are:

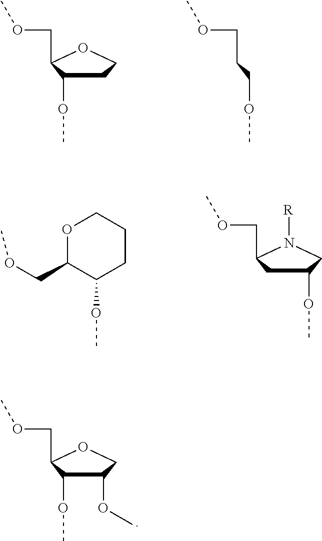

Exemplified sugar modifications are:

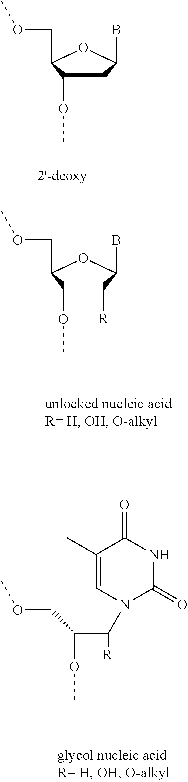

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

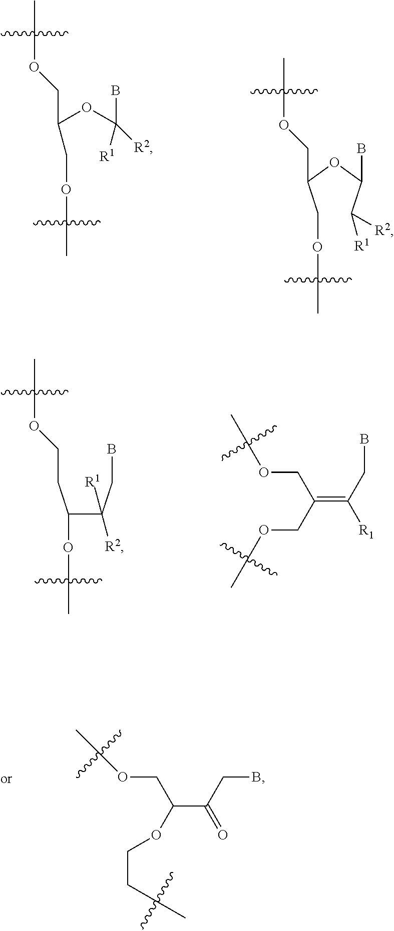

wherein B is a modified or unmodified nucleobase, R¹ and R² independently are H, halogen, OR₃, or alkyl; and R³ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

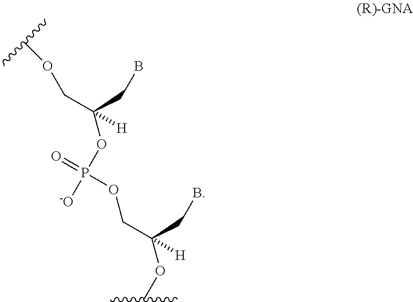

The thermally destabilizing modification can be mismatches (i.e., noncomplementary base pairs) between the thermally destabilizing nucleotide and the opposing nucleotide in the opposite strand within the sciRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

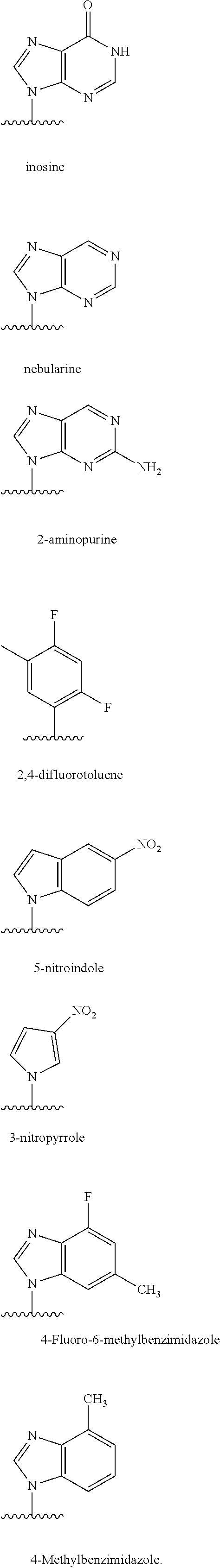

Exemplary phosphate modifications known to decrease the thermal stability of dsRNA duplexes compared to natural phosphodiester linkages are:

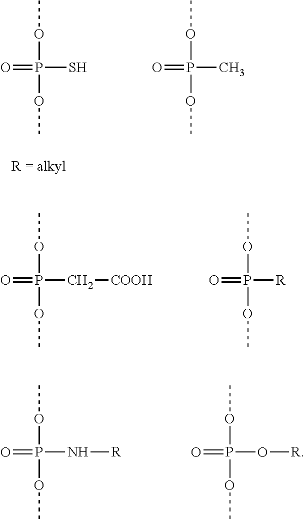

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In some embodiments, at least one strand of the sciRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P—5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for sciRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the sciRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, 1-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a sciRNA agent of the invention that modulates a micro-RNA.

In some embodiments, the invention provides a sciRNA agent for extrahepatic delivery, and target a CNS gene or ocular gene.

In some embodiments, provided herein is a sciRNA that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, provided herein is a sciRNA agent that targets TARDBP for ALS, MAPT (Tau) for Frontotemporal Dementia, and HTT for Huntington Disease.

In some embodiments, provided herein is a sciRNA agent that targets SNCA for Parkinson Disease, FUS for ALS, ATXN3 for Spinocerebellar Ataxia 3, ATXN1 for SCA1, genes for SCAT and SCAB, ATN1 for DRPLA, MeCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

Additional examples of CNS gene and ocular gene can be found in WO 2019/217459, which is incorporated herein by reference in its entirety.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dsiRNA compounds.

In an alternative functional assay, a candidate dsiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dsiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The sciRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an sciRNA compound on a human, e.g., any unwanted effect, such as a toxic effect, or any positive, or desired effect.

Increasing Cellular Uptake of siRNAs

Described herein are various sciRNA compositions that contain covalently attached conjugates that increase cellular uptake and/or intracellular targeting of the sciRNAs.

Additionally provided are methods of the invention that include administering an sciRNA compound and a drug that affects the uptake of the sciRNA into the cell. The drug can be administered before, after, or at the same time that the sciRNA compound is administered. The drug can be covalently or non-covalently linked to the sciRNA compound. The drug can be, for example, a lipopolysaccharide, an activator of p38 MAP kinase, or an activator of NF-κB. The drug can have a transient effect on the cell. The drug can increase the uptake of the sciRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the sciRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

sciRNA Production

An sciRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove and siRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

Making Double-Stranded sciRNA Agents Conjugated to a Ligand

In some embodiments, a ligand can be conjugated to the sciRNA agent via a nucleobase, sugar moiety, internucleosidic linkage, or a carrier, as described herein.

Detailed methods for conjugating a ligand to an siRNA agent (such as a lipophilic moiety or a carbohydrate-based ligand) can be found in WO 2019/217459, WO 2009/082607, and WO 2009/073809, all of which are incorporated herein by reference in their entirety.

### Pharmaceutical Compostions

In one aspect, the invention features a pharmaceutical composition that includes an sciRNA compound, e.g., a double-stranded siRNA compound, or ssiRNA compound, (e.g., a precursor, e.g., a larger siRNA compound which can be processed into a ssiRNA compound, or a DNA which encodes an sciRNA compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the pharmaceutical composition can be an emulsion, microemulsion, cream, jelly, or liposome.

In one example, the pharmaceutical composition includes an sciRNA compound mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an sciRNA compound admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an sciRNA compound in a form suitable for oral delivery. In one embodiment, oral delivery can be used to deliver an sciRNA compound composition to a cell or a region of the gastro-intestinal tract, e.g., small intestine, colon (e.g., to treat a colon cancer), and so forth. The oral delivery form can be tablets, capsules or gel capsules. In one embodiment, the sciRNA compound of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an sciRNA compound and a delivery vehicle.

In one embodiment, the delivery vehicle can deliver an sciRNA compound to a cell by a topical route of administration. The delivery vehicle can be microscopic vesicles. In one example the microscopic vesicles are liposomes. In some embodiments the liposomes are cationic liposomes. In another example the microscopic vesicles are micelles.

In one aspect, the invention features a pharmaceutical composition including an sciRNA compound in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an sciRNA compound in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In one aspect, the invention features a pharmaceutical composition including an sciRNA compound in a rectal dosage form. In one embodiment, the rectal dosage form is an enema. In another embodiment, the rectal dosage form is a suppository.

In one aspect, the invention features a pharmaceutical composition including an sciRNA compound in a vaginal dosage form. In one embodiment, the vaginal dosage form is a suppository. In another embodiment, the vaginal dosage form is a foam, cream, or gel.

In one aspect, the invention features a pharmaceutical composition including an sciRNA compound in a pulmonary or nasal dosage form. In one embodiment, the sciRNA compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the sciRNA agent of the invention. In one embodiment, the cell is a heptic cell. In one embodiment, the cell is an extraheptic cell.

Another aspect of the invention relates to a method of reducing the expression of a target gene in a subject, comprising administering to the subject the sciRNA agent of the invention.

The sciRNA agent of the invention can be delivered to a subject by a variety of routes, depending on the type of genes targeted and the type of disorders to be treated. In some embodiments, the sciRNA agent is administered hepatically. In some embodiments, the sciRNA agent is administered extrahepatically, such as an ocular administration (e.g., intravitreal administration) or an intrathecal administration.

In one embodiment, the sciRNA agent is administered intrathecally. By intrathecal administration of the sciRNA agent, the method can reduce the expression of a target gene in a brain or spine tissue, for instance, cortex, cerebellum, cervical spine, lumbar spine, and thoracic spine.

For ease of exposition the formulations, compositions and methods in this section are discussed largely with regard to modified sciRNA compounds. It may be understood, however, that these formulations, compositions and methods can be practiced with other siRNA compounds, e.g., unmodified sciRNA compounds, and such practice is within the invention. A composition that includes a iRNA can be delivered to a subject by a variety of routes. Exemplary routes include: intravenous, topical, rectal, anal, vaginal, nasal, pulmonary, ocular.

The sciRNA molecules of the invention can be incorporated into pharmaceutical compositions suitable for administration. Such compositions typically include one or more species of sciRNA and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the sciRNA compound composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the sciRNA agent is delivered by intrathecal injection (i.e. injection into the spinal fluid which bathes the brain and spinal chord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal chord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Rectal Administration. The invention also provides methods, compositions, and kits, for rectal administration or delivery of sciRNA compounds described herein.

Accordingly, an sciRNA compound can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the sciRNA compound is by means of an enema. The sciRNA compound of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The sciRNA agents described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the sciRNA agents may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the sciRNA agents may be administered into the eye, for example the vitreous chamber of the eye, by intravitreal injection, such as with pre-filled syringes in ready-to-inject form for use by medical personnel.

For ophthalmic delivery, the sciRNA agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the sciRNA agents. Viscosity building agents, such as hydroxymethyl cellulose, hydroxyethyl cellulose, methylcellulose, polyvinylpyrrolidone, or the like may be added to the pharmaceutical compositions to improve the retention of the sciRNA agents.

To prepare a sterile ophthalmic ointment formulation, the sciRNA agents is combined with a preservative in an appropriate vehicle, such as mineral oil, liquid lanolin, or white petrolatum. Sterile ophthalmic gel formulations may be prepared by suspending the sciRNA agents in a hydrophilic base prepared from the combination of, for example, CARBOPOL®-940 (BF Goodrich, Charlotte, N.C.), or the like, according to methods known in the art.

Topical Delivery. Any of the sciRNA compounds described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the sciRNA compound composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

In some embodiments, an sciRNA compound is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the sciRNA compounds described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. sciRNA compounds can be formulated as a liquid or nonliquid, e.g., a powder, crystal, or aerosol for pulmonary delivery.

A composition that includes an sciRNA compound can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the sciRNA compounds described herein can be administered orally, e.g., in the form of tablets, capsules, gel capsules, lozenges, troches or liquid syrups. Further, the composition can be applied topically to a surface of the oral cavity.

Any of the sciRNA compounds described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue sciRNA compounds can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of the sciRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

### Kits

In certain other aspects, the invention provides kits that include a suitable container containing a pharmaceutical formulation of an sciRNA compound. In certain embodiments the individual components of the pharmaceutical formulation may be provided in one container. Alternatively, it may be desirable to provide the components of the pharmaceutical formulation separately in two or more containers, e.g., one container for an sciRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention, and are not intended to limit the invention.

### Example 1: Synthesis of Small Circular Interfering RNA (sciRNA) GalNAc Conjugates, and In Vitro and In Vivo Gene Silencing

### Synthesis of Oligonucleotides

Oligonucleotides were synthesized on a MerMade-12 DNA/RNA synthesizer. Sterling solvents/reagents from Glen Research, 500-Å controlled pore glass (CPG) solid supports (Prime Synthesis), 2′-deoxy 3′-phosphoramidites (Thermo), and 2′-O-methyl (2′-OMe), 2′-deoxy-2′-fluoro (2′-F) ribonucleoside 3′-phosphoramidites (Hongene) were all used as received. The 2′-OMe-uridine-5∝-bis-POM-(E) vinylphosphonate (VP) 3′-phosphoramidite (synthesized according to the procedures described in Parmar et al., “Facile synthesis, geometry, and 2′-substituent-dependent in vivo activity of 5′-(E)- and 5′-(Z)-vinylphosphonate-modified siRNA conjugates,” J Med. Chem., 61: 734-44 (2018), which is incorporated herein by reference in its entirety) was dissolved to 0.15 M in 85% acetonitrile 15% dimethylformamide (DMF) and coupled using standard conditions on the synthesizer. GalNAc CPG support (L, Scheme I) was prepared and used as described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety. 5-Bromohexyl phosphoramidite (Glen Research, Cat #10-1946) was dissolved to 0.15 M in acetonitrile and coupled using standard conditions on the synthesizer. 3′-alkyne CPG support and 3′-alkyne hydroxyprolinol phosphoramidite (Y, Scheme I) was prepared and used as described in Jayaprakash et al., “Non-nucleoside building blocks for copper-assisted and copper-free click chemistry for the efficient synthesis of RNA conjugates,” Org. Lett., 12: 5410-13 (2010), which is incorporated herein by reference in its entirety. Low-water content acetonitrile was purchased from EMD Chemicals. DNA and RNA oligonucleotides were synthesized using modified synthesis cycles, based on those provided with the instrument. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M 12 in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase syntheses (SPS), the CPG solid support was washed with 5% (v/v) piperidine in anhydrous acetonitrile three times with 5-minute holds after each flow. The support was then washed with anhydrous acetonitrile and dried with argon. The oligonucleotides were then incubated with 28-30% (w/v) NH₄OH at 35° C. for 20 hours. For VP-containing oligonucleotides, after completion of the SPS, the CPG solid support was incubated with 28-30% (w/v) NH₄OH, where 5% of (v/v) of diethylamine was added at 35° C. for 20 hours (see O'Shea et al., “An efficient deprotection method for 5′-[O,O-bis(pivaloyloxymethyl)]-(E)-vinylphosphonate containing oligonucleotides,” Tetrahedron, 74: 6182-86 (2018), which is incorporated herein by reference in its entirety). The solvent was collected by filtration and the support was rinsed with water prior to analysis. Oligonucleotide solutions of ˜1 OD₂₆₀ units/mL were used for analysis of the crudes, where 30-50 μL of solution were injected. LC/ESI-MS was performed on an Agilent 6130 single quadrupole LC/MS system using an XBridge C8 column (2.1×50 mm, 2.5 μm) at 60° C. Buffer A consisted of 200 mM 1,1,1,3,3,3-hexafluoro-2-propanol (HFIP) and 16.3 mM triethylamine (TEA) in water, and buffer B was 100% methanol. A gradient from 0% to 40% of buffer B over 10 minutes followed by washing and recalibration at a flow rate of 0.70 mL/min. The column temperature was 75° C.

All oligonucleotides were purified and desalted, and further annealed to form GalNAc-siRNAs, based on the procedures described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety.

Circulation of Sense-Strand Oligonucleotides and Formation of sciRNA-GalNAc Conjugates 5′-azido and “Click” Chemistry Synthesis for Circular Oligonucleotides.

To functionalize the sense strand, commercially available 5-bromohexyl phosphoramidite was coupled to the 5′ end during solid-phase synthesis. To provide the 6-azidohexyl click chemistry “handle”, 10 μmol of CPG loaded with 5′-(5-bromohexyl) modified oligonucleotides (see Lietard et al., “An efficient reagent for 5′-azido oligonucleotide synthesis,” Tetrahedron Lett., 48, 8795-98 (2007), which is incorporated herein by reference in its entirety) were suspended in 15 mL of an anhydrous DMF solution containing 130 mg of sodium azide and 300 mg of sodium iodide. The mixture was vigorously shaken at 65° C. for 75 minutes. After cooling down, the solution was filtered off and the CPG beads with the resulting 5′-(5-azidohexyl) solid-supported oligonucleotides were washed with DMF (2×10 mL) and dried under a stream of argon. The oligonucleotides were released from the solid support and purified and desalted as described above. The oligonucleotides were then dissolved in water to a concentration of ˜10 OD₂₆₀ units/mL. For a copper(I)-catalyzed alkyne-azide cycloaddition (CuAAC) “click” cyclization reaction yielding Z (Scheme I), 2 mL of the oligonucleotide solution (˜200 OD₂₆₀ units) were mixed with 2 mL of methanol, 1.1 mL of sodium L-ascorbate (0.1 mM) and 1.1 mL of copper sulfate (20 mM). The reaction mixture was placed into a microwave (MW) tube container, equipped with a stirring bar, and placed in a MW reactor for 40 minutes at 60° C. (power ˜8 W, stirring, cooling, P═0). This protocol is a modification of that described in Lietard et al., “New strategies for cyclization and bicyclization of oligonucleotides by click chemistry assisted by microwaves,” J. Org. Chem., 73, 191-200 (2008), which is incorporated herein by reference in its entirety. All cyclic oligonucleotides were purified and desalted, and further annealed with an antisense strand to form the GalNAc-sciRNA as described above.

### Enzymatic Stability Assays

3′ Exonuclease SVPD stability assay. Modified oligonucleotide was added at 0.1 mg/mL to a solution of 50 mM Tris-HCl (pH 7.2) and 10 mM MgCl₂. Snake Venom Phosphodiesterase (SVPD) (Worthington, Cat #LS003926) was added to the mixture at 750 mU/mL. Immediately after addition of the enzyme, the sample was injected onto a Dionex DNAPac PA200 column (4 mm×250 mm) at 30° C. and run at a flow rate of 1 mL/minute with a gradient of 40-55% Buffer B over 7.5 minutes. Buffer A was 20 mM sodium phosphate, 15% acetonitrile, pH 11; and Buffer B was Buffer A containing 1 M sodium bromide (pH 11). Aliquots were analyzed every hour for 24 hours. The area under the peak corresponding to full-length oligonucleotide was normalized to the area from the 0-hour time point (first injection). First order decay kinetics were assumed in calculation of half-lives. A control oligonucleotide, dT₁₉·dT (where dT is 2′-deoxythymidine, and · is a single 3′-terminal phosphorothioate linkage) was analyzed each day, and half-lives were reported relative to half-life of the control sequence. Enzyme was prepared as a stock of 1000 mU/mL aliquoted into 1 mL tubes and stored at −20° C. A new aliquot was used each week. Experiments were performed in triplicates.

5′ Exonuclease Phosphodiesterase II stability assay. Modified oligonucleotide was added at 0.1 mg/mL to a solution of 50 mM sodium acetate (pH 6.5) and 10 mM MgCl₂. Phosphodiesterase II from bovine spleen (Worthington, Cat #LS003 602) was added to the mixture at 500 mU/mL. Immediately after addition of the enzyme, the sample was injected onto a Dionex DNAPac PA200 column (4 mm×250 mm) at 30° C. and run at a flow rate of 1 mL/minute with a gradient of 37-52% Buffer B over 7.5 minutes. Buffer A was 20 mM sodium phosphate, 15% acetonitrile, pH 11; and Buffer B was Buffer A containing 1 M sodium bromide (pH 11). Aliquots were analyzed every hour for 24 hours. The area under the peak corresponding to full-length oligonucleotide was normalized to the area from the 0-hour time point (first injection). First order decay kinetics were assumed in calculation of half-lives. A control oligonucleotide, dT·dT₁₉ (where dT is 2′-deoxythymidine, and is a single 5′-terminal phosphorothioate linkage) was analyzed each day, and half-lives were reported relative to half-life of the control sequence. Enzyme was prepared as a stock of 2000 mU/mL, aliquoted into 1 mL tubes and stored at −20° C. A new aliquot was used each week. Experiments were performed in triplicates.

### Analysis of Oligonucleotide Stability in Plasma and Liver Homogenates

Rat plasma (BioIVT, Cat #RAT00PL38NCXNN) and liver homogenate (BioIVT, custom order) were diluted with a 10× cofactor solution to achieve a final concentration of 1 mM MgCl₂, 1 mM MnCl₂, and 2 mM CaCl₂. The sciRNA was added to 50 μL of the plasma or the liver homogenate to achieve the final concertation of 20 μg/mL. The reaction mixture was incubated with gently shaking at 37° C. At each predetermined time point (0, 1, 4, 8, and 24 hour), the reaction was stopped by adding 450 μL of Clarity OTX lysis-loading buffer (Phenomenex, Cat #AL0-8579) containing internal standard (oligonucleotide U₂₁ at 1 μg/mL final concentration) and frozen at −80° C. until analysis. Experiments were performed in triplicate.

Oligonucleotide enrichment for LC-MS analysis was performed using Clarity OTX 96-well solid-phase extraction plates as described by Liu et al., “Oligonucleotide quantification and metabolite profiling by high-resolution and accurate mass spectrometry,” Bioanalysis, 11: 1967-80 (2019), which is incorporated herein by reference in its entirety. The SPE columns were conditioned initially with 1 mL of methanol followed by equilibration with 2 mL of 50 mM ammonium acetate with 2 mM sodium azide in HPLC-grade water. The samples were loaded on to SPE column by applying positive pressure. The columns were then washed five times with 1 mL of 50 mM ammonium acetate in 50/50 (v/v) water and acetonitrile (pH 5.5). Finally, the oligonucleotides are eluted using elution buffer containing 10 mM EDTA, 100 mM ammonium bicarbonate in 40/10/50 (v/v/v) acetonitrile/tetrahydrofuran/water (pH 8.8). The eluant was dried under nitrogen and resuspended in 120 μL of LC-MS grade water for LC-MS analysis.

Relative quantitation and metabolite identification of modified oligonucleotides was performed using high-resolution mass spectrometry on a Thermo Scientific Q Exactive coupled to ion-pairing reverse-phase liquid chromatography (Dionex Ultimate 3000) (LC-HRMS). A Waters X-Bridge BEH C8 XP Column (Cat #176002554,130 Å, 2.5 μm, 2.1 mm×30 mm, 80° C.) was used for the chromatographic separation. The injection volume and flow rates were 30 μL and 1 mL/min, respectively. Mobile phase A consisted of 16 mM triethylamine (Sigma, Cat #471283), 200 mM 1,1,1,3,3,3-hexafluoro-2-propanol (Fisher, Cat #67-56-1) in LC-MS grade water (Fisher, Cat #7732-18-5); mobile phase B was 100% methanol (Fisher, Cat #67-56-1). The gradient started with 1% mobile phase B and progressed to 35% B over 4.3 minutes, then the column was equilibrated with 1% mobile phase B for 1 minute. The mass spectrometer data acquisition was performed in full scan mode with a scan range of 500-3000 m/z at a resolution setting of 35,000. Spray voltage was 2.8 kV. The auxiliary gas temperature and the capillary temperature were set to 300° C.

The Thermo Quan browser was used calculate the area ratio of extracted ion chromatograms (XIC) of test oligonucleotide to internal standard with 10 ppm mass accuracy. The m/z ions for used for XIC of the test oligonucleotide were 1018.7752, 1018.8868, 1019.1101, 1019.2220, 1019.3325, 1019.4436, 1019.5559, and 1019.6671, and m/z ions used for XIC of the internal standard were 1258.5073, 1258.8408, 1259.1740, 1259.8411, 1289.5061. After LC-HRMS analysis, data were processed using ProMass HR Deconvolution software (Novatia, LLC) to identify linearization and major metabolism of the modified oligonucleotides as described in Liu et al., “Oligonucleotide quantification and metabolite profiling by high-resolution and accurate mass spectrometry,” Bioanalysis, 11: 1967-80 (2019), which is incorporated herein by reference in its entirety.

The half-lives were calculated by monitoring loss of full-length test oligonucleotide for 24 hours, based on the method described in Chan et al., “Meeting the Challenge of Predicting Hepatic Clearance of Compounds Slowly Metabolized by Cytochrome P450 Using a Novel Hepatocyte Model, HepatoPac,” Drug Metab. Dispos. 41: 2024-32 (2013), which is incorporated herein by reference in its entirety. The amounts of test oligonucleotide and internal standard were normalized to time 0 hour for each time-point for respective oligonucleotides. The natural log of percentage full length remaining and the slope were calculated using linear regression. The half-life was calculated using equation (1):

$${t}_{\frac{1}{2}} = - \frac{Ln ( 2 )}{k} . \qquad ( 1 )$$

### Thermal Melting Studies

Melting studies were performed in 1 cm path length quartz cells on a Beckman DU800 spectrophotometer equipped with a thermoprogrammer. Duplexes were diluted to obtain a final concentration of ˜1 μM in 0.1× PBS buffer (pH 7.4) by adding 16 μL of stock solution of duplexes (1 mg/mL in 1.0× PBS) to 1 mL of 0.1× PBS buffer (pH 7.4). Melting curves were monitored at 260 nm with a heating rate of 1° C./minute from 10 to 90° C. Melting temperatures (Tₘ) were calculated from the first derivatives of the heating curves (in built software) and the reported values are the result of two independent measurements.

### NMR Studies

Lyophilized RNA was dissolved in a mixture of 10% ²H₂O/90% H₂O with 20 mM NaCl and 10 mM sodium phospate buffer (pH 7). Equimolar ratios of sense and antisense strands were mixed to constitute linear or circular duplexes. Final concentrations of duplexes in 600 μL were in the range from 20 to 60 μM. All spectra were acquired at 25° C. on an Agilent VNMRS 800 MHz NMR spectrometer equipped with a cold probe.

### In Vitro Silencing Activity Studies

Transfection. Primary Mouse Hepatocytes (PMH) were transfected with siRNA to test for silencing efficiency. siRNA (5 μL) at the indicated concentrations was mixed with 4.9 μL of Opti-MEM and 0.1 μL of Lipofectamine RNAiMax (Invitrogen, Cat #13778-150) per well of a 384-well plate and incubated at room temperature. After 15 minutes, 40 μL of William's E medium or EMEM medium containing approximately 5×10³ cells were added to the wells. Cells were incubated for 24 hours prior to RNA purification.

Free uptake. Free uptake experiments were carried out in PMH. siRNA (5 μL at the indicated concentration) was mixed with 5 μL of Opti-MEM per well of a 384-well plate. After 15 min, 45 μL of William's E medium or EMEM medium containing approximately 5×10³ cells were added to the wells. Cells were incubated for 48 hours prior to RNA purification.

Total RNA isolation using Dynabeads mRNA isolation kit. RNA was isolated using an automated protocol on a BioTek-EL406 platform using Dynabeads (Invitrogen, Cat #61012). 50 μL of lysis/binding buffer (Tris HCl pH 7.5, LiCl, EDTA pH 8.0, DTT) and 25 μL of lysis/binding buffer containing 3 μL of magnetic beads were added to each well. The plates were incubated on an electromagnetic shaker for 10 minutes at room temperature, then the magnetic beads were captured, and the supernatant was removed. The bead-bound RNA was washed twice with 150 μL/well of Buffer A (Tris HCl pH 7.5, LiCl, EDTA pH 8.0, DTT), and then washed once with 150 μL/well of Buffer B (Tris HCl pH 7.5, LiCl, EDTA pH 8.0). The beads were then washed with 150 μL of Elution Buffer and re-captured, and the supernatant was collected.

cDNA synthesis using ABI High-capacity cDNA reverse transcription kit. cDNA synthesis was performed using an ABI kit (Cat #4368813). To the wells of a 384-well plate containing the RNA isolated using Dynabeads was added 10 μL of a master mix containing 1 μL 10×Buffer, 0.4 μL 25×dNTPs, 1 μL 10×random primers, 0.5 μL reverse transcriptase, 0.5 μL RNase inhibitor and 6.4 μL of nuclease free water. The plates were sealed, mixed, and incubated on an electromagnetic shaker for 10 minutes at room temperature, followed by 2 hours at 37° C.

Gene expression analysis (RT-qPCR). All probes for RNA quantification were acquired from Life Technologies utilizing their Taqman gene expression system with dual labeled probes which allowed for analysis of gene expression. Target gene expression was normalized to the Gapdh ubiquitous control in each well utilizing a dual label system. Ct values were measured using a Light Cycler 480 (Roche). To calculate relative fold change, real time data were analyzed using the ΔΔCt method and normalized to assays performed with cells treated with a non-targeting siRNA control. Taqman probe catalogue numbers: Mouse C5 (Mm00439275_m1), Mouse Gapdh 4352339E, Mouse Ttr (Mm00443267_m1).

### In Vivo Studies of Silencing Activity in Mice

All procedures using mice were conducted by certified laboratory personnel using protocols consistent with local, state, and federal regulations. Experimental protocols were approved by the Institutional Animal Care and Use Committee (IACUC), the Association for Assessment and Accreditation of Laboratory Animal Care International (accreditation number: 001345), and the office of Laboratory Animal Welfare (accreditation number: A4517-01). The number of samples for animal studies was determined to be one that would allow for confidence in the resulting data set utilizing the least number of animals, as required in accordance with IACUC guidelines. All animals were acclimated in-house for 48 hours prior to study start. Female C57BL/6 mice approximately 8 weeks of age were obtained from Charles River Laboratories and randomly assigned to each group. All animals were treated in accordance with IACUC protocols.

Animals were dosed subcutaneously at 10 μL/g with sciRNA, siRNA duplex, or with PBS saline control. Doses used in this study were 3 mg/kg. The sciRNAs and siRNAs were diluted into phosphate buffered saline (PBS, pH 7.4). All dosing solutions were stored at 4° C. until time of injection. Animals were sacrificed at either 5 or 7 days post dose. Livers were harvested and snap frozen for analysis.

Serum collection. Blood was collected utilizing the retro-orbital eye bleed procedure 24 h post the final dose in accordance with the IACUC approved protocol. The sample was collected in Becton Dickinson serum separator tubes (Fisher Scientific, Cat #BD365967). For analysis of TTR, serum samples were kept at room temperature for 1 hour and then spun in a micro-centrifuge at 21,000×g at room temperature for 10 minutes. Serum was transferred into 1.5 mL micro-centrifuge tubes for storage at −80° C. until the time of assay. Serum collected for the analysis of circulating C5 was kept at room temperature for 15 minutes, and then immediately transferred to 4° C. prior to spinning in a micro-centrifuge at 21,000×g at room temperature for 10 minutes. Serum was transferred into 1.5 mL micro-centrifuge tubes for storage at −80° C. until the time of assay.

Circulating serum transthyretin (TTR) levels. Serum samples were diluted 1:4,000 and assayed using a commercially available kit from ALPCO specific for detection of mouse prealbumin (Cat #41-PALMS-E01). Protein concentrations (μg/mL) were determined by comparison to a purified TTR standard, following the manufacturer's instructions.

Circulating serum complement 5 (C5) levels. An ELISA assay was developed to specifically detect circulating mouse C5 levels. The primary antibody was goat-anti-human C5 (Complement Technologies, Cat #A220), and the secondary antibody was bovine anti-goat IgG-HRP (Jackson ImmunoResearch, Cat #805-035-180), which had minimal cross-reactivity to other species. Antibodies were used at 0.8 mg/mL. The assay was developed using a TMB substrate kit (R&D Systems, Cat #DY999), and the reaction was stopped using sulfuric acid prior to measurement. The serum samples were diluted 1:5,000 for analysis.

### In Vivo Liver Exposure and Ago2 Loading

Mice were sacrificed on day 7 post-dose. Livers were snap frozen in liquid nitrogen and ground into powder for further analysis. Total siRNA liver levels were measured by reconstituting liver powder at 10 mg/mL in PBS containing 0.25% Triton-X 100. The tissue suspension was further ground with 5-mm steel grinding balls at 50 cycles/s for 5 minutes in a tissue homogenizer (Qiagen TissueLyser LT) at 4° C. Homogenized samples were then heated at 95° C. for 5 minutes, briefly vortexed, and allowed to rest on ice for 5 minutes. Samples were then centrifuged at 21,000×g for 15 minutes at 4° C. The siRNA-containing supernatants were transferred to new tubes. The siRNA sense and antisense strand levels were quantified by stem loop reverse transcription followed by Taqman PCR (SL-RT QPCR)(see the methods described in Chen et al., “Real-time quantification of microRNAs by stem-loop RT-PCR,” Nucleic Acids Res., 33: e179-e179 (2005); and Pei et al., “Quantitative evaluation of siRNA delivery in vivo,” RNA, 16: 2553-2563 (2010), which are incorporated by reference in their entirety) and adapted to chemically modified siRNAs.

Ago2-bound siRNA from mouse liver was quantified by preparing liver powder lysates at 100 mg/mL in lysis buffer (50 mM Tris-HCl, pH 7.5, 150 mM NaCl, 2 mM EDTA, 0.5% Triton-X 100) supplemented with freshly added protease inhibitors (Sigma-Aldrich, Cat #P8340) at 1:100 dilution and 1 mM PMSF (Sigma-Aldrich, Cat #P7626). Total liver lysate (10 mg) was used for each Ago2 immunoprecipitation (IP) and control IP. The siRNA not bound to Ago2 was cleaned up with pre-saturated QAE Resin (GE healthcare, Cat #17-0200-01) in lysis buffer (16 mg/ml) supplemented with protease inhibitors at 1:100 dilution and 1 mM PMSF. Samples were filtered through cellulose acetate filter (Fisher, Cat #P169702) to remove the resin before proceeding with the Ago2 IP. Anti-Ago2 antibody was purchased from Wako Chemicals (Clone No.: 2D4). Control mouse IgG was from Santa Cruz Biotechnology (Cat #sc-2025). Protein G Dynabeads (Life Technologies, Cat #10003D) were used to precipitate antibodies. Ago2-associated siRNAs were eluted by heating (50 μL PBS, 0.25% Triton X-100; at 95° C. for 5 minutes) and quantified by SL-RT QPCR (see the methods described in Chen et al., “Real-time quantification of microRNAs by stem-loop RT-PCR,” Nucleic Acids Res., 33: e179-e179 (2005); and Pei et al., “Quantitative evaluation of siRNA delivery in vivo,” RNA, 16: 2553-2563 (2010), which are incorporated by reference in their entirety).

### Molecular Modeling

Coordinates of the complex between human Ago2 and an antisense (guide, AS): passenger (sense, S) strand duplex with seed region pairing were retrieved from the Protein Data Bank (www.rcsb.org; ID code 4W5T) (36). In the complex, the S strand is comprised of residues S1 to S9 (5′ to 3′ direction). The Z linker was built with the program UCSF Chimera (37) using the structure-editing ‘build structure’ and ‘modify structure’ tools and starting from the 3′-terminus of the sense strand (S9). After adding a 3′-phosphate group, the linker was constructed using ideal bond lengths and angles and an extended conformation in the direction of the 5′-end of the S strand wherever possible, while avoiding short contacts with AS strand and Ago2 side chains of the Ago2 PIWI and MID domains and L2 linker region. The 5′-terminal S1 residue as observed in the crystal structure of the complex was looped around in order to get its 5′-hydroxyl group to point in the direction of the growing Z linker. After addition of a phosphate to the last methylene carbon of the linker, a distance of some 5 Å remained between 5′-OH of S1 and phosphorous. This distance was systematically shortened by altering S1 and linker torsion angles until a final distance of 1.6 Å allowed cyclization of the sense strand with acceptable bond and torsion angles.

### Chemical Synthesis of GalNAc-sciRNA Conjugates.

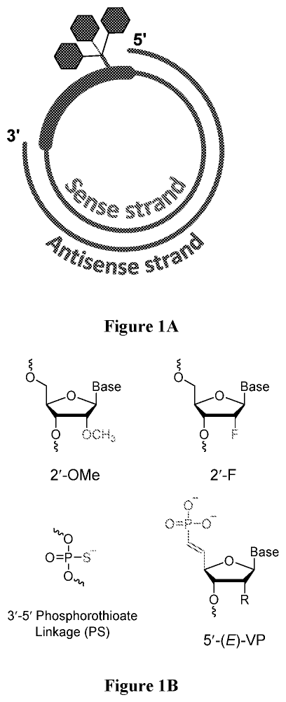

In this example, sciRNA constructs have been designed where the sense strand was cyclized and was conjugated at the 3′-end to a trivalent GalNAc ligand. As shown in FIG. 1A, the cyclized sense strand was then annealed to a linear antisense strand. Both sense and antisense strands were constructed with 2′-OMe, 2′-F, and PS backbone modifications, as shown in FIG. 1B.

The cyclization procedure is exemplified in Scheme I. As shown in Scheme I, an additional “click” chemistry “handle,” an N-alkyne linker as a hydroxyprolinol building block (Y) attached directly to the GalNAc ligand (L) solid support (see Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated by reference in its entirety), was introduced at the 3′-end of the sense strand (see Jayaprakash et al., “Non-nucleoside building blocks for copper-assisted and copper-free click chemistry for the efficient synthesis of RNA conjugates,” Org. Lett., 12: 5410-13 (2010), which is incorporated by reference in its entirety), via its phosphoramidite, in a standard solid-phase synthesis (SPS) setup. Both the hydroxyprolinol linker and the trivalent GalNAc ligand are clinically validated moieties in RNAi therapeutics.

After completion of the SPS of the entire sense stand sequence, the commercially available 6-bromohexyl phosphoramidite was coupled to the 5′-end of the sense strand. Subsequently, the bromine (1, Scheme I) was substituted with an azido group (2, Scheme I), providing the 6-azidohexyl handle (Q) (see Lietard et al., “An efficient reagent for 5′-azido oligonucleotide synthesis,” Tetrahedron Lett., 48: 8795-98 (2007), which is incorporated by reference in its entirety). After cleavage from the solid support, deprotection and purification of the full-length 5′-azido oligonucleotide, the copper(I)-catalyzed alkyne-azide cycloaddition (CuAAC) “click” reaction allowed for the completion of cyclized, circular sense strands of the sciRNA (see Lietard et al., “New strategies for cyclization and bicyclization of oligonucleotides by click chemistry assisted by microwaves,” J. Org. Chem., 73: 191-200 (2008); and Huisgen, “Kinetics and reaction mechanisms: selected examples from the experience of forty years,” Pure and Appl. Chem., 61: 613-28 (1989), both of which are incorporated by reference in their entirety). Annealing with the corresponding complementary antisense strands yielded the target sciRNA duplexes.

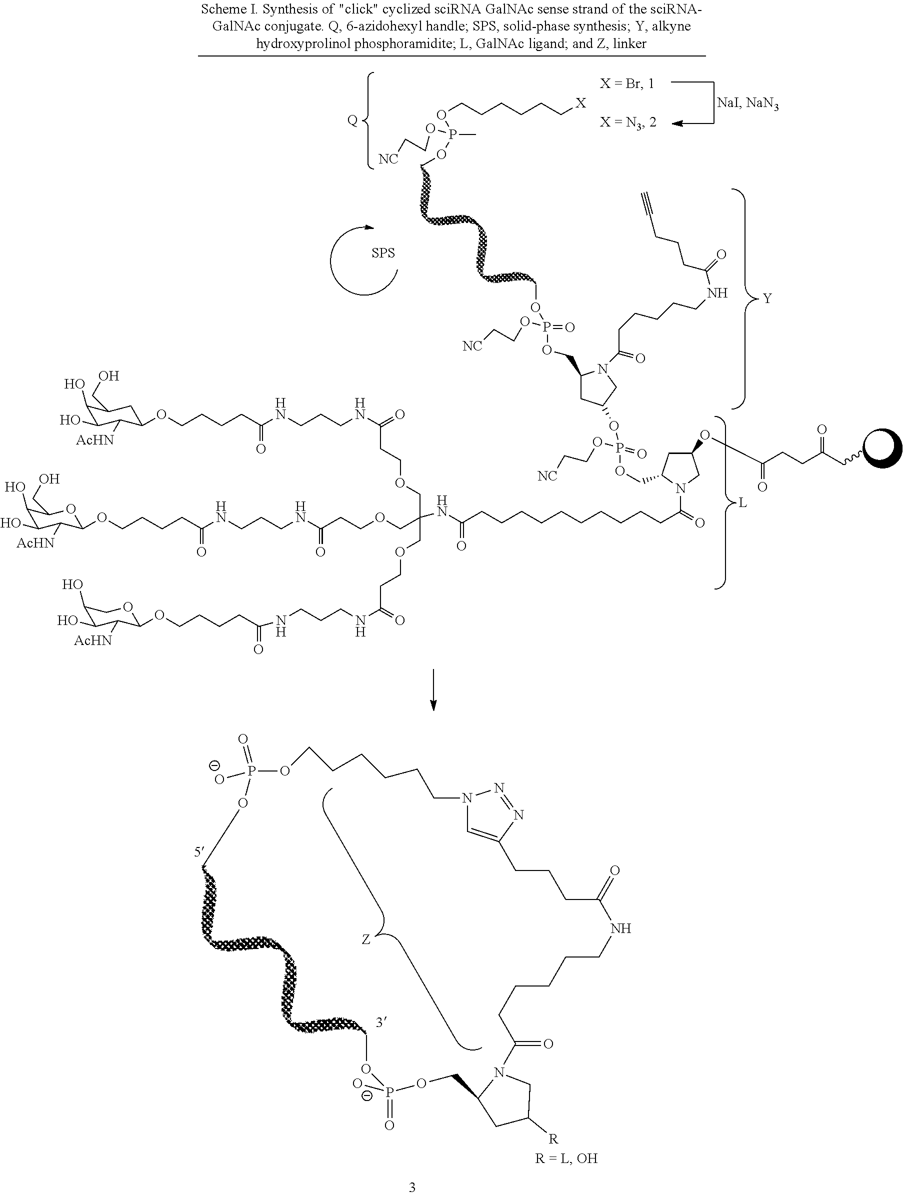

The oligonucleotides used in this example are listed and characterized in Table 1.

TABLE 1
Oligonucleotide sequences and mass spectra-based characterization

| | | | Sense/ | Calc | Found |
|---|---|---|---|---|---|
| Oligonucleotide | Sequences (5′-3′)^{a} | Duplex | Antisense | MW | MW |
| ON-1 | (dT)₁₉•dT | | | 6037.96 | 6036.95 |
| ON-2 | dT•(dT)₁₉ | | | 6037.96 | 6037.11 |
| ON-3 | Q(dT)₂₃Y | | | 7526.02 | 7524.37 |
| ON-4 | Z(dT)₂₃ | | | 7526.02 | 7524.47 |
| ON-5 | QaaAaCaGuGuUCUuGcUcUaUaAY | | | 8047.08 | 8045.22 |
| ON-6 | ZaaAaCaGuGuUCUuGcUcUaUaA | | | 8047.08 | 8044.98 |
| ON-7 | A•a•CaGuGuUCUuGcUcUaUaAL | si-1 | Sense | 8590.17 | 8588.69 |
| ON-8 | u•U•aUaGaGcAagaAcAcUgUu•u•u | si-1/si-3/ | Antisense | 7595.94 | 7594.79 |
| | | si-4 | | | |
| ON-9 | VPu•U•aUaGaGcAagaAcAcUgUu•u•u | si-2/si-5 | Antisense | 7672.94 | 7670.55 |
| ON-10 | QA•a•CaGuGuUCUuGcUcUaUaAYL | si-3 | Sense | 9181.71 | 9179.98 |
| ON-11 | ZA•a•CaGuGuUCUuGcUcUaUaAL | si-4/si-5 | Sense | 9181.71 | 9179.71 |
| ON-12 | G•a•CaAaAuAACuCaCuAuAaUL | si-6 | Sense | 8627.29 | 8626.45 |
| ON-13 | a•U•uAuAgUgAguuAuUuUgUc•a•a | si-6/si-7 | Antisense | 7574.87 | 7573.88 |
| ON-14 | ZG•a•CaAaAuAACuCaCuAuAaUL | si-7 | Antisense | 9218.83 | 9217.13 |

^{a}Chemistry modifications are indicated as follows: •, PS linkage; lower case nucleotides, 2′-OMe; upper case nucleotides in italics, 2′-F; Q, 6-azidohexyl-phosphate; VP, 5′-(E)-vinylphosphonate. For structures of L, Y, and Z, see Scheme I. Z denotes a cyclic oligonucleotide.

### Enzymatic Stability of Cyclic Oligonucleotides.

To assess the stabilization that the cyclic modification Z can impart to the oligonucleotide nuclease resistance, in vitro assay using either a 3′-exonuclease or 5′-exonuclease, paired with their appropriate assay controls, were utilized. The results of the enzymatic stability of linear and circular single-stranded oligonucleotides, compared against the controls (ON-1 and ON-2) are shown in Table 2.

TABLE 2
Enzymatic stability of linear and circular single-stranded oligonucleotides.

| | | 3′- | Intact | 5′- | Intact |
|---|---|---|---|---|---|
| | | Exonuclease | after 24 | Exonuclease | after 24 |
| Oligonucleotide | Sequences (5′-3′)^{a} | half-life^{b} | h (%) | half-life^{c} | h (%) |
| ON-1/ON-2 | (dT)₁₉•dT/dT•(dT)₁₉ | 1 | 10.0 | 1 | 6.5 |
| ON-3 | Q(dT)₂₃Y | 0.7 | 2.7 | 8.2 | 13.3 |
| ON-4 | Z(dT)₂₃ | 1.2 | 14.8 | 26.8 | 52.5 |
| ON-5 | QaaAaCaGuGuUCUuGcUcUaUaAY | 12.8 | 76.1 | 14.1 | 83.8 |
| ON-6 | ZaaAaCaGuGuUCUuGcUcUaUaA | 14.5 | 79.1 | 423.0 | 100.0 |

^{a}Chemistry modifications legend: •—phosphorothioate (PS) linkage; dT—2′-deoxythimidine; lower case nucleotides—2′-O-methyl (OMe); upper case nucleotides in italics—2′-deoxy-2′-fluoro (F); Q = 6-azidohexyl-phosphate; structures of Y and Z—see Scheme I; and Z denotes a cyclic oligonucleotide.
^{b}Half-life value determined in hours and reported relative to that of the (dT)₁₉•dT control.
^{c}Half-life value was determined in hours and reported relative to that of the dT•(dT)₁₉ control. First order decay kinetics were assumed in calculation of half-lives. The data is representative of two independent experiments with similar results.

As shown in Table 2, single-stranded 23-mer phosphodiester oligonucleotides were composed either of full 2′-deoxythymidine (ON-3 and ON-4) or of fully chemically 2′-modified nucleotides (ON-5 and ON-6). Each series included the circular oligonucleotide strands containing the “clicked” Z chemical modification (ON-4 and ON-6), as well as the linear oligonucleotides containing the “unclicked” precursor moieties Q and Y (ON-3 and ON-5).

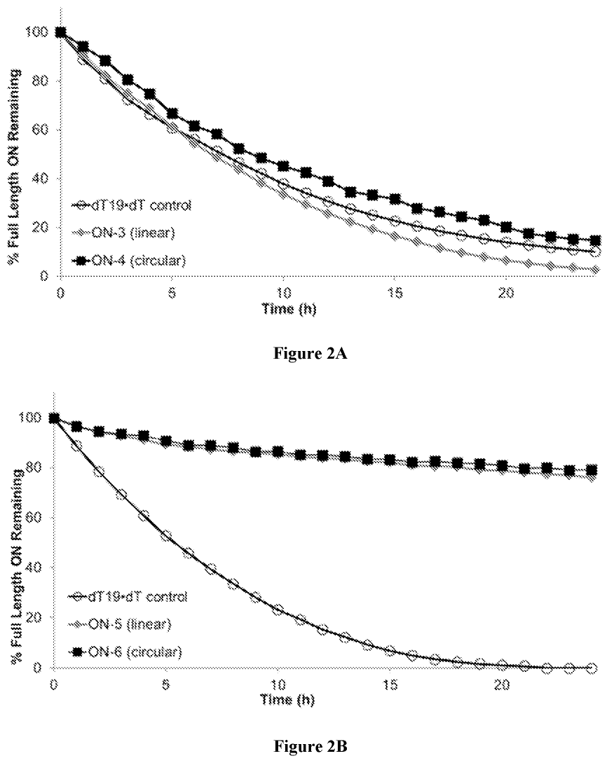

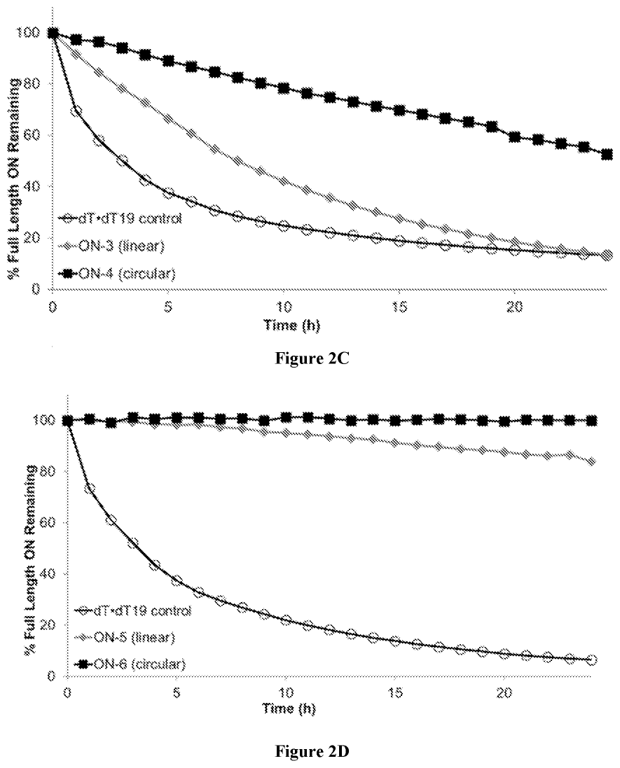

As shown in FIGS. 2A-2D, the results from the exonuclease assays revealed that the cyclic modification Z provided higher exonuclease stability than the assay controls as well as the linear counterparts. ON-4 (deoxythymidine) and ON-6 (chemically modified), were more stable than the linear counterparts ON-3 and ON-5, respectively) and than the control oligonucleotides (ON-1 and ON-2). The stabilization was even further enhanced by the introduction of the fully chemically 2′-modified nucleotides in the modified series, where the cyclized, fully chemically 2′-modified oligonucleotide ON-6 was virtually stable to exonuclease action, i.e., not degraded to a significant extent by either nuclease over the time course of the experiment (FIG. 2D). In the unmodified 2′-deoxy series (ON-3 and ON-4), the cyclic modification Z provided higher stabilization towards 5′-exonuclease (FIG. 2C) than towards 3′-exonuclease (FIG. 2A). Likewise, the 3′-exonuclease assays showed less differentiation between linear and circular oligonucleotides than the 5′-exonuclease (FIGS. 2A and 2C).

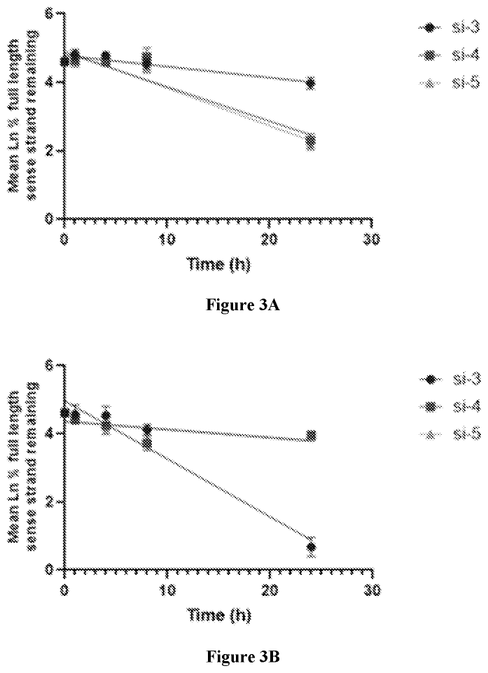

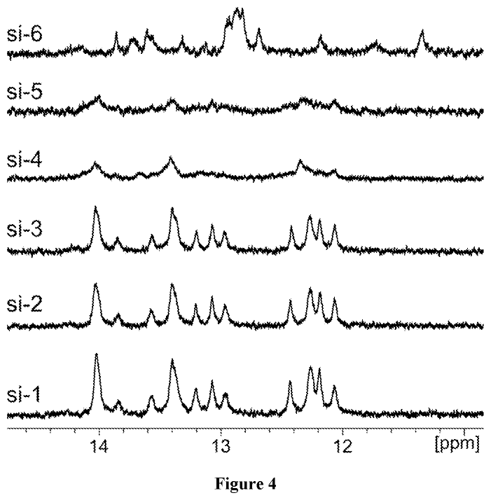

To study the metabolism of the circular siRNA designs, the stability studies were extended from the individual single strands to the annealed sciRNA duplexes. The in vitro stabilities of two sciRNAs (si-4 and si-5, see Table 4 below) and the linear siRNA containing the same nucleotide sequence and modifications (si-3) were assessed in rat plasma and rat liver homogenates. The results are shown in FIGS. 3A-3B. Metabolite identifications of si-3, si-4, and si-5 based on the LC-MS analysis from rat liver homogenate or rat plasma were summarized in Table 3A, Table 3B, and Table 3C, respectively.

As shown in FIGS. 3A-3B, no degradation of either the circular sciRNA strands or the linear siRNA strands was detected in plasma for up to 8 hours (FIG. 3A). However, in metabolite analysis by LC-MS, after 24 hours of incubation in rat plasma, an addition of a water molecule to the circular strand was observed (Tables 3B and 3C). This may have resulted from opening of the cyclic structure, yielding in linearized oligonucleotide. In rat liver homogenates, the circular sciRNAs (si-4 and si-5) were more stable than the linear counterpart (si-3). There was no degradation of si-4 or si-5 at 24 hours, whereas for si-3, only ˜55% of the full-length sense strand was detected at 24 hour (FIG. 3B). The sciRNAs have significantly longer half-lives of 29 and 30 hours compared to the linear siRNA, which has a 4-hour half-life. This correlates with the results of the analysis of the stabilities of the single strands, where circularization enhanced stability in the presence of exonucleases. In the liver homogenates, opening of the cyclic structure of the sense strands of either si-4 or si-5 was not observed (Tables 3B and 3C).

TABLE 3A
Metabolite identification of si-3 from rat liver homogenate or plasma

| Observed | | | |
|---|---|---|---|
| mass | Possible metabolite sequence | Strand | Description ^{a} |
| 7592.52 | U*U*aUaGaGcAagaAcAcUgUu*u*u | Antisense | Parent |
| 6588.965 | UaGaGcAagaAcAcUgUu*u*u | Antisense | 5’ n-4 |
| 6371.919 | Pu*U*aUaGaGcAagaAcAcUg | Antisense | 3’ n-4 |
| 6291.957 | U*U*aUaGaGcAagaAcAcUg | Antisense | 3’ n-4 |
| 5700.846 | VPu*U*aUaGaGcAagaAcAc | Antisense | 3’ n-5 |
| 9177.039 | QA*a*CaGuGuUCUuGcUcUaUaAL | Sense | Parent |
| 8973.962 | ZA*a*CaGuGuUCUuGcUcUaUaAL | Sense | -GalNAc₁ |
| | (-GalNAc₁) | | |

^{a} -GalNAc1 refers to loss of single GalNAc sugar from the triantenary ligand.

TABLE 3B
Metabolite identification of si-4 from rat liver homogenate or plasma

| Observed | | | |
|---|---|---|---|
| mass | Proposed metabolite sequence | Strand | Description ^{a} |
| 7592.52 | U*U*aUaGaGcAagaAcAcUgUu*u*u | Antisense | Parent |
| 6588.965 | UaGaGcAagaAcAcUgUu*u*u | Antisense | 5’ n-4 |
| 6291.957 | U*U*aUaGaGcAagaAcAcUg | Antisense | 3’ n-4 |
| 9177.039 | QA*a*CaGuGuUCUuGcUcUaUaAYL | Sense | Parent |
| 9195.049 | QA*a*CaGuGuUCUuGcUcUaUaAYL | Sense | Hydrolyzed |

^{a} Hydrolyzed = parent strand +18.015 Da.

TABLE 3C
Metabolite identification of si-5 from rat liver homogenate or plasma

| Observed | | | |
|---|---|---|---|
| mass | Proposed metabolite sequence | Strand | Description ^{a} |
| 7668.030 | VPU*U*aUaGaGcAagaAcAcUgUu*u*u | Antisense | Parent |
| 6588.965 | VPu*U*aUaGaGcAagaAcAcUgP | Antisense | 5′ n-4 |
| 6447.9166 | Pu*U*aUaGaGcAagaAcAcUg | Antisense | 3′ n-3 |
| 5700.846 | QA*a*CaGuGuUCUuGcUcUaUaAYL/ | Antisense | 3′ n-5 |
| | ZA•a•CaGuGuUCUuGcUcUaUaAL | | |
| 9177.039 | QA*a*CaGuGuUCUuGcUcUaUaAYL | Sense | Parent |
| 9195.049 | Z49A*a*CaGuGuUCUuGcUcUaUaAL | Sense | Hydrolyzed |
| 8973.962 | Z49A*a*CaGuGuUCUuGcUcUaUaAL | Sense | -GalNAc₁ |
| | (-GalNAc₁) | | |

^{a} Hydrolyzed = parent strand +18.015 Da.
-GalNAc1 refers to loss of single GalNAc sugar from triantenary ligand.
Z49—cyclization by clicking 3′-phosphate-Hyp-C9-1,4-triazole-C6-5′-phosphate:

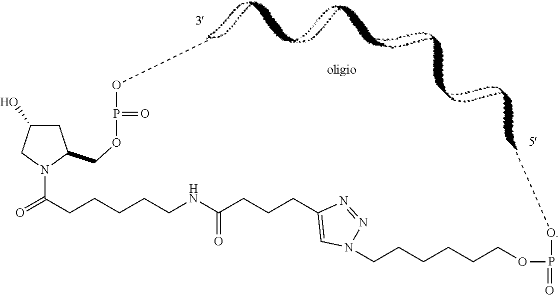

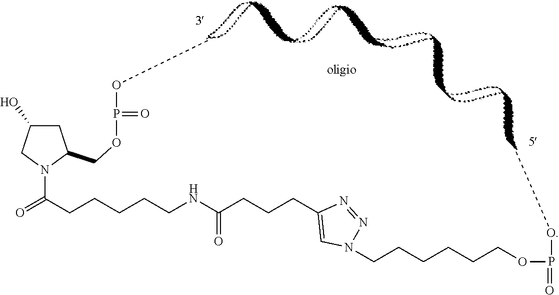

### Thermal Stability of GalNAc-sciRNA Conjugates and NMR Studies.

Table 4 shows the thermal stability of the circular GalNAc-sciRNA conjugates and linear GalNAc-siRNA conjugates. The cyclic 21-nt sense strands (siRNAs sense strands containing the Z modification) were hybridized with their respective complementary 23-nt antisense strands in order to prepare the sciRNA duplexes, and the melting temperature (Tₘ) of each complex was determined. For comparison, the Tₘ of the open (i.e., not circular) siRNA duplexes were also determined. These included the standard GalNAc-siRNA constructs formed from linear sense strands (si-1, si-2 and si-6), but also one representative siRNA formed from linear sense strand (si-3), where the 5′ and 3′ sense strand modifications Q and Y, have not been engaged in the “click” reaction yielding the cyclic triazole Z moiety connecting the 5′ and 3′ ends of the siRNA sense strand.

As shown in Table 4, the sciRNA constructs (si-4, si-5 and si-7) showed an increased destabilization (by 15-20° C.) compared to the open counterpart (si-3) or standard siRNA duplexes. This thermal destabilization points to a strain of the cyclic 21-nt sense strand, which can impart flexibility in the siRNA duplex structure, and a subset of the nucleobases form Watson-Crick base pairs.

The solution-state ¹H NMR analysis of the siRNA duplexes in FIG. 4 show that the spectra of linear siRNA duplexes (Table 4, si-1, si-2, si-3 and si-6) exhibit well-resolved resonances. Several signals for imino proton could be observed in the region from δ 12 to 14 ppm, which is indicative of Watson-Crick base pairing. The number of signals suggests fully complementary duplexes are formed. The spectra of circular sciRNA duplexes (Table 4, si-4 and si-5) exhibit broad signals. But a few signals were observed in the imino regions, indicating a partial duplex structure, suggesting some thermal destabilization and a subset of the nucleobases form Watson-Crick base pairs.

TABLE 4
Thermal stability and in vitro potency in primary mouse hepatocytes (PMHs) of linear
GalNAc-siRNA and circular GalNAc-sciRNA conjugates.

| | | | Tₘ (° C.)^{b} | Transfection | Free uptake |
|---|---|---|---|---|---|
| siRNA | Target | Sequences (5′-3′)^{a} | (ΔTₘ)^{c} | IC₅₀ (pM)^{d} | IC₅₀ (pM)^{e} |
| si-1 | Ttr | A•a•CaGuGuUCUuGcUcUaUaAL | 68.2 | 151.1 ± 21.8 | 53.1 ± 15.6 |
| | | u•U•aUaGaGcAagaAcAcUgUu•u•u | | | |
| si-2 | Ttr | A•a•CaGuGuUCUuGcUcUaUaAL | 67.8 | 41.9 ± 13.8 | 21.6 ± 3.3 |
| | | VPu•U•aUaGaGcAagaAcAcUgUu•u•u | (−0.4) | | |
| si-3 | Ttr | QA•a•CaGuGuUCUuGcUcUaUaAYL | 67.3 | 81.3 ± 55.5 | 58.5 ± 1.9 |
| | | u•U•aUaGaGcAagaAcAcUgUu•u•u | (−0.9) | | |
| si-4 | Ttr | ZA•a•CaGuGuUCUuGcUcUaUaAL | 52.4 | 836.8 ± 140.4 | 197.9 ± 31.2 |
| | | u•U•aUaGaGcAagaAcAcUgUu•u•u | (−15.7) | | |
| si-5 | Ttr | ZA•a•CaGuGuUCUuGcUcUaUaAL | 52.5 | 124.7 ± 64.1 | 63.9 ± 9.1 |
| | | VPu•U•aUaGaGcAagaAcAcUgUu•u•u | (−15.7) | | |
| si-6 | C5 | G•a•CaAaAuAACuCaCuAuAaUL | 62.3 | 60.5 ± 30.1 | 109.2 ± 53.8 |
| | | a•U•uAuAgUgAguuAuUuUgUc•a•a | | | |
| si-7 | C5 | ZG•a•CaAaAuAACuCaCuAuAaUL | 41.8 | 830.2 ± 195.0 | 824.4 ± 72.7 |
| | | a•U•uAuAgUgAguuAuUuUgUc•a•a | (−20.5) | | |

^{a}Sense strand sequences are the top rows; antisense strand sequences are the bottom rows. Chemistry modifications legend: •—phosphorothioate (PS) linkage; lower case nucleotides—2′-O-methyl (OMe); upper case nucleotides in italics—2′-deoxy-2′-fluoro (F); sense strands (top rows); antisense strands (bottom rows); Q—6-azidohexyl-phosphate; structures of L, Y and Z—see Scheme I; VP refers to 5′-(E)-vinylphosphonate; and Z denotes a cyclic oligonucleotide.
^{b}Tₘ refers to the melting temperature obtained from the maxima of the first derivatives of the melting curves (A₂₆₀ vs temperature) recorded in 0.1 × PBS buffer (pH 7.4) using 1.0 μM concentrations of each strand.
^{c}ΔTₘ refers to the change in melting temperature compared to the unmodified duplex (for si-2 through si-5, reference duplex is si-1, and for entry si-7, reference duplex is si-6). Average of two independent annealing sample preparations and measures (n = 2).
^{d}Compounds were added to PMHs by transfection with Lipofectamine RNAiMAX and after a 24 h incubation, cells were lysed and processed for RNA isolation, cDNA synthesis, and quantitative PCR analysis. mRNA levels were normalized relative to mouse Gapdh. Mean IC₅₀ values reported with SD (n = 4).
^{e}Compounds were added to PMHs by free uptake and after a 48 h incubation, cells were lysed and processed for RNA isolation, cDNA synthesis, and quantitative PCR analysis. mRNA levels were normalized relative to mouse Gapdh. Mean IC₅₀ values reported with SD (n = 4).

### In Vitro and In Vivo Activity of GalNAc-sciRNA Conjugates.

Several sciRNA designs were evaluated and potent gene expression silencing in vitro and in vivo were observed, albeit in some cases lower than that of the non-circular GalNAc-siRNA parents.

The in vitro potency of the sciRNAs was determined in primary mouse hepatocytes (PMHs), after either transfection of the siRNAs using Lipofectamine RNAi Max, or after simple free uptake within the PMHs via the GalNAc ligand and its uptake receptor, the asialoglycoprotein receptor (ASGPR), which is ubiquitously expressed on the surface of hepatocytes. The results of in vitro potency of linear GalNAc-siRNA and circular GalNAc-sciRNA conjugates in PMHs are shown in Table 4. Cyclic sciRNA constructs were prepared targeting two distinct targets, namely the mRNAs of rodent transthyretin (Ttr) or complement 5 (C5), alongside the standard GalNAc-siRNAs and the corresponding linear “unclicked” siRNA controls, having the Q and Y chemical modifications resulting in the cyclic “click” construct Z. Table 4 shows that using each of the two modes of cellular delivery, strong inhibition of target mRNA expression were observed in all cases, albeit with a loss of potency in the in vitro inhibition of mRNA levels with the cyclic sciRNAs, compared to either linear counterparts.

As shown in Table 4, in the Ttr series, relative to the linear controls si-1 and si-3, the cyclic sciRNA compound si-4 had a 5-fold (transfection) and 4-fold (free uptake) loss of in vitro potency, correlating with the thermal destabilization observed in the Tₘ studies.

In the same context, the use of the 5′-(E)-vinylphosphonate VP modification of the antisense strands, a modification that can enhance the metabolic stability and Ago2 loading of siRNAs, were evaluated by preparing the control VP siRNA (si-2) and the cyclic sciRNA (si-5). By introducing the VP modification, the in vitro potency of the sciRNA conjugates was further enhanced and the potency gap between the linear and circular Ttr-targeting siRNAs was reduced to only 3-fold. There was an about 3-fold potency improvement triggered by the introduction of the VP modification as shown by comparison of the two control Ttr-targeting siRNAs (si-1 and si-2). The cyclic sciRNA with a VP-modified sense strand (si-5) was only 3-fold less potent than the VP-modified control with a linear sense strand (si-2), and displayed similar potency as the linear control (si-1).

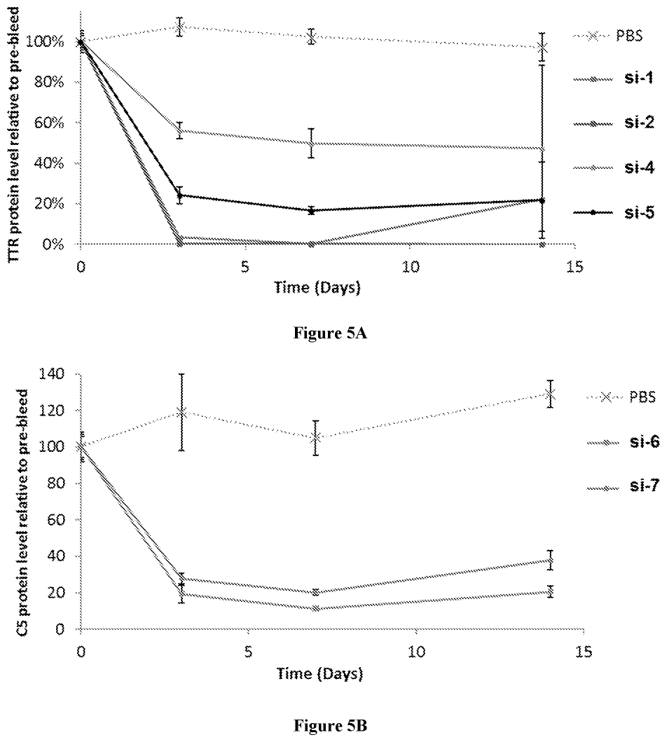

The GalNAc-sciRNAs activities were also evaluated in vivo. For this experiment, all GalNAc conjugates (including siRNAs and linear counterparts) were subcutaneously administered as a single dose (3 mg/kg on Day 0), and the serum levels of circulating TTR or C5 proteins were determined over time for each treatment group. The results of pharmacodynamics profiles after a single subcutaneous administration of linear GalNAc-siRNA and circular GalNAc-sciRNA conjugates in mice are shown in FIGS. 5A-5B. In FIGS. 5A-5B, both linear and circular designs showed a strong target inhibition in vivo. For the Ttr siRNA series, the data in mice mirrored the in vitro results as shown in Table 4, where a reduction of siRNA efficacy over the linear controls (si-1 and si-2) was observed with the sciRNA molecules (si-4 and si-5, FIG. 5A). For the series targeting C5, the identical 3 mg/kg dose of the linear (si-6) and circular (si-7) siRNA conjugates resulted in similar potency of reducing circulating C5 protein in mice (FIG. 5B), whereas at least an ˜8-fold potency loss was observed in vitro (Table 4).

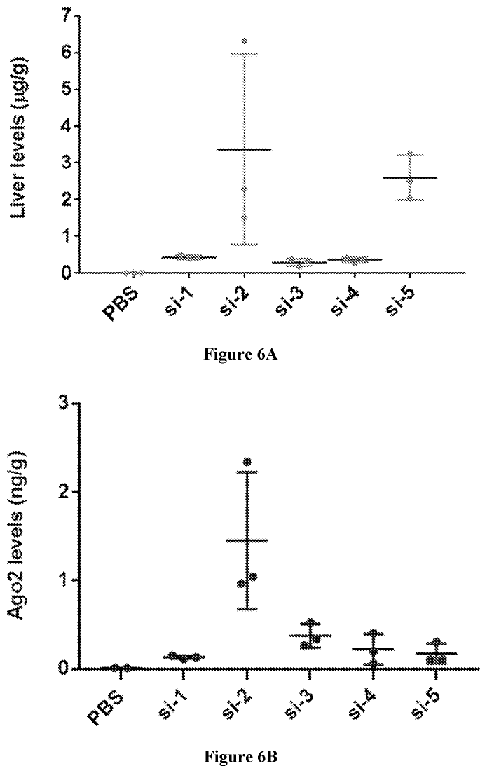

The in vivo metabolic stability of sciRNA compounds was further evaluated by measuring the total liver levels and Ago2-loaded levels, following a SC administration of a 3 mg/kg dose in mice. The results are shown in FIGS. 6A-6B. Mouse livers were harvested at Day 7 and treated to isolate and quantify the antisense strand of each siRNA by SL-RT QPCR. Relative liver amounts for the antisense strands measured for siRNAs si-1-si-5 are shown in FIG. 6A, showing that the addition of the metabolically stable VP group significantly increased the liver levels of both the corresponding linear siRNA (si-2) and circular sciRNA (si-5), over the non-VP siRNAs (˜5-fold more potent than respective controls with a linear sense strand si-1 and si-4, respectively). The whole liver levels between si-2 and si-5 were comparable (FIG. 6A), whereas their pharmacodynamics profiles in mice differed significantly (see FIG. 5A).

To evaluate the efficiency of Ago2 loading, Ago2 was immunoprecipitated from mouse whole liver lysate, and the antisense strand levels from Ago2 were extracted and measured, with the results shown in FIG. 6B. The VP modification in the linear siRNA enhanced Ago2 loading of the (si-2) antisense strand, relative to the non-VP siRNA (si-1) (FIG. 6B). The sciRNA VP counterpart (si-5) did not exhibit the same level of Ago2 loading enhancement, relative to the non-VP sciRNA (si-3) (FIG. 6B). A similar trend of reduction in Ago2 loading can be noted between the non-VP linear siRNA si-3 and the sciRNA si-4 (FIG. 6B). Linear siRNA (si-2) and circular sciRNA (si-5) exhibited similar antisense strand liver levels (FIG. 6A) but had different levels of Ago2-loaded antisense strand (FIG. 6B). This demonstrates that cyclic GalNAc-sciRNAs exhibited a metabolic stability similar to linear GalNAc-siRNAs, with a lower loading efficiency of antisense strands into Ago2.

These examples described the solid phase synthesis of a new class of fully chemically modified small circular interfering RNAs (sciRNAs) with a “click”-cyclized sense (S) strand, carrying a trivalent GalNAc ligand and an alkyne-functionalized linker. After completion of the sense strand synthesis, commercial bromo reagents were coupled at the 5′-end, and the bromide group of the linker was substituted with an azido group (Scheme I). After cleavage from the solid support, deprotection and purification, “click” reaction conditions allowed for the cyclization of the sense strands. Annealing of the circular sense strands with the corresponding complementary antisense (AS) strands yielded the GalNAc-conjugated sciRNA duplexes.

Physicochemical and pharmacological properties that these novel designs impart to GalNAc-sciRNAs were studied. Cyclization of a ˜20-nt oligonucleotide provided stability towards nucleases, but could reduce the Tₘ and thereby the thermal stability of the resulting RNA duplexes (see Tables 2 and 4). Annealing of shorter oligonucleotide targets (n=6-nt to 10-nt) to a 20-nt cyclic oligonucleotide showed increase in Tₘ values, demonstrating that a shorter linear target can be structurally well accommodated with a cyclic DNA strand. Furthermore, a triplex structure, where the cyclic 20-nt partner binds to two 10-nt strands can benefit from the cyclic partner being able to form Hoogsteen hydrogen bounds and lowers the entropy of the structure. The annealing of the 20-nt cyclic oligonucleotide with its 20-nt target displayed the same Tₘ value as that with the 10-nt target, suggesting that −10 nucleotides of the target were able to hybridize with the cyclic oligonucleotide. For DNA, longer nucleotide cycles may help form more stable circular duplexes. See Kumar et al., “Template-directed oligonucleotide strand ligation, covalent intramolecular DNA circularization and catenation using click chemistry,” J. Am. Chem. Soc., 129: 6859-64 (2007), which is incorporated herein by reference in its entirety.

The thermal melting and NMR analysis of the sciRNAs designed to form 21 base pairs indicate that when the sense strand has a rigid circular structure, a subset of the nucleobases form Watson-Crick base pairs. Fewer and broadened NMR signals were observed in spectra of sciRNAs than linear siRNAs suggesting that, in the sciRNAs, a heterogeneous mixture of partially base paired duplexes coexists in solution. The 21/23-nt sciRNA exhibited a circular sense strand structure, and when annealing with the linear antisense strand, a subset of the nucleobases formed Watson-Crick base pairs, which could impart a thermal destabilization to the duplex.

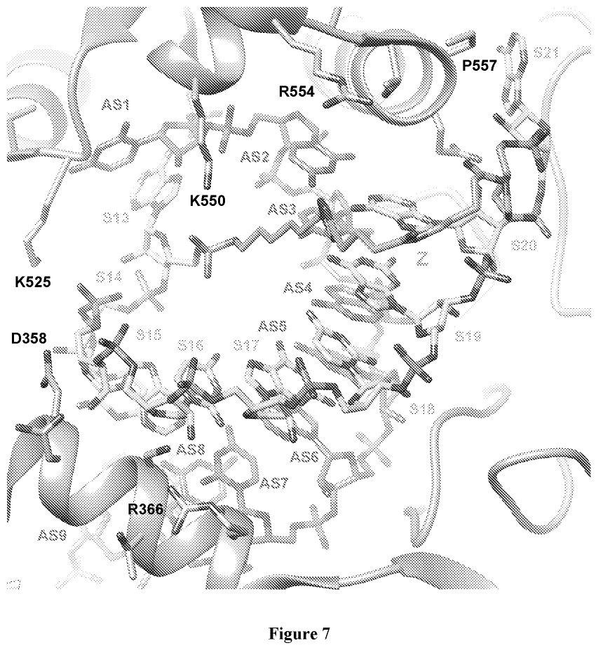

FIG. 7 shows a molecular model of an sciRNA:Ago2 complex based on the crystal structure of Ago2 bound to duplex RNA with seed region pairing. As shown in FIG. 7, the model of an sciRNA:Ago2 complex confirms that cyclization of the sense strand (S) with the Z linker maintained base pairing in the entire seed region (AS2:S20-AS8:S14). The crystal structure of Ago2 bound to AS and S RNA strands was used to construct the sciRNA scenario. See Schirle et al., “Structural basis for microRNA targeting,” Science, 346: 608-13 (2014), which is incorporated herein by reference in its entirety. Thus, the Z linker connects the 3′-terminal S strand nucleotide (A9 in the crystal structure, and labeled S21 in FIG. 7, consistent with the length of S strands listed in Table 4) and the first S strand residue visible in the crystal structure (A1, labeled S13 in the model). The latter residue was flipped around to complete the loop and close the circle, with the Z linker crossing the major groove of the S:AS duplex. Pairing across the entire seed region seen in the model is consistent with the increased Tₘ values observed for shorter oligonucleotides targeted with a 20-nt cyclic strand. Based on this model, a bigger circle with all 21 passenger strand nucleotides circularized by Z (+12 nt compared to the S13-S21 model depicted in FIG. 7) may be generated. Although, based on this model, the Z linker could presumably bridge across 8 or few pairs and, thus, after four or five more base pairs there would need a loop to connect the beginning of the sense strand back to the Z linker. The prolinol moiety adjacent to the 3′-phosphate of residue S21 in this model was oriented such that the hydroxyl group juts outwards, thus placing the GalNAc ligand on the surface of the complex and precluding potential clashes with Ago2 (FIG. 7).

The circular GalNAc-sciRNAs in this experiment demonstrated potent gene expression silencing in vitro and in vivo (Table 4, FIGS. 5A-5B). The thermal destabilization and duplex strain induced by the cyclization of the sense strand in sciRNAs showed an impact on the inherent potency of RNAi-mediated silencing, which is typically measured in vitro, especially when using transfection reagents for intracellular uptake.

However, the potency difference between linear GalNAc-siRNAs and circular GalNAc-sciRNAs was generally less pronounced when the constructs were evaluated in mice than in cell culture (FIGS. 5A-5B). The discrepancy between in vitro and in vivo potency could be the result of increased impact of metabolic stability on potency (efficacy in vivo) which outweighs intrinsic RNAi silencing potency. For example, in the Ttr series (FIG. 5A), the addition of the stabilizing VP group in the sciRNA (si-5) had a greater fold impact on in vivo potency than in vitro potency. In mice, at Day 14 post dose, the sciRNA with an antisense strand modified with VP had a potency equivalent to that of the linear non-VP-modified siRNA (si-1).

In rat plasma, neither sense nor antisense strands of sciRNAs or siRNAs were degraded in plasma at 8 hours. Considerable linearization of the circular sense strand of the sciRNA was observed after 24 hours of incubation. This indicates that the circular structure can act as a pro-drug, with hydrolysis (presumably of a phosphate group), resulting in a linear oligonucleotide. This hydrolysis did not occur when the compounds were incubated in rat liver homogenates. GalNAc-conjugated siRNA is typically rapidly absorbed into liver through efficient ASGPR-mediated uptake. Thus, linearization may have an impact on the in vivo potency due to rapid uptake of GalNAc-siRNA by hepatocytes.

The molecular model in FIG. 7 supports the loading of the sciRNA onto Ago2 by preserving the seed region nucleotide base pairing between the two RNA strands. The model also highlights important structural changes that may require the sciRNA duplex to flex and result in a thermodynamically destabilized duplex structure. In terms of intrinsic RNAi potency, the partial duplex structure shown in the model positions these sciRNA constructs somewhere between canonical siRNA duplexes and single-stranded siRNAs (ss-siRNAs). For the latter, loss of potency between 10- and 100-fold, compared to the siRNA duplex counterparts, has been reported (see Lima et al., “Single-stranded siRNAs activate RNAi in animals,” Cell, 150: 883-94 (2012); Prakash et al., “Lipid nanoparticles improve activity of single-stranded siRNA and gapmer antisense oligonucleotides in animals,” ACS Chem. Biol., 8: 1402-06 (2013), which are incorporated by reference in their entirety). To this date, no therapeutic development clinical candidate has been reported using the ss-siRNA platform. Moreover, the use of VP modifications in the sciRNAs provided potency benefit to the sciRNAs.

The results here also indicate that the sense strand played a role for RNAi activity for loading of the duplex into Ago2. The sense strand also had a role as being the carrier for the GalNAc ligand. The successful delivery of GalNAc-sciRNAs to hepatocytes demonstrates that the cyclic sense strand is complexed with the antisense strand despite the relative instability. Additionally, this confirms that the GalNAc ligand can effectively deliver RNAi therapeutics cargo when placed internally as well as terminally.

This work is the first example of partial-duplex sciRNA constructs that are functionally effective at pharmacologically and therapeutically relevant doses in vivo, useful for therapeutic siRNA molecules having enhanced potency resulting from increased metabolic stability and decreased off-target properties.

### Example 2: Synthesis of Circular Bis-sciRNA

Circular bis-sciRNAs were synthesized in this example, the sequences are shown in the table below.

| Multi- | | | | | | | | |
|---|---|---|---|---|---|---|---|---|
| plex | Oligo | | | | Oligo | | | |
| ID | ID | Sequence (5′ to 3′)^{a} | Strand | Target | ID | Sequence (5′ to 3′) | Strand | Target |
| AM- | A- | usascug(Uhd)UfgGfAf | sense | CTNNB1/SOD | | | | |
| 206 | 2832990 | Ufugauucgasasauuucsa | | 1 | | | | |
| | | suuuuAfaUfCfCfucacu | | | | | | |
| | | cuasasZ84 | | | | | | |
| | A- | VPusUfsucgAfaUfCfa | antis | CTN | A- | VPusUfsuagAfgUfGfa | antis | SOD1 |
| | 141361 | aucCfaAfcaguasgsc | | NB1 | 44440 | ggaUfuAfaaaugsasg | | |
| | | | | | 2 | | | |
| AM- | A- | usascuguUfgGfAfUfu | sense | CTNNB1/SOD | | | | |
| 210 | 2832991 | gauucgasasauuucsasuu | | 1 | | | | |
| | | uuAfaUfCfCfucacucua | | | | | | |
| | | sasZ83L96 | | | | | | |
| | A- | VPusUfsucgAfaUfCfa | antis | CTN | A- | VPusUfsuagAfgUfGfa | antis | SOD1 |
| | 141361 | aucCfaAfcaguasgsc | | NB1 | 44440 | ggaUfuAfaaaugsasg | | |
| | | | | | 2 | | | |

^{a} Chemistry modifications are indicated as follows: s, PS linkage; lower case nucleotides, 2′-OMe; upper case nucleotides followed by f (Uf, Gf, Cf, Af), 2′-F; VP, 5′-(E)-vinylphosphonate; (Uhd) is 2′-O-hexadecyl-uridine-3′-phosphate. For structures of Z83 and Z84, see Z in Scheme I, which denotes a cyclized sense strand.
Z83 indicates a cyclized sense strand via 3′ internal cyclic click of Apy and Q301: 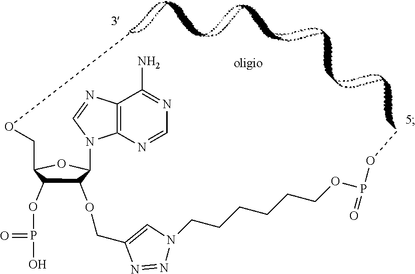
Z84 indicates a cyclized sense strand via 3′ terminal cyclic click of Apy and Q301: 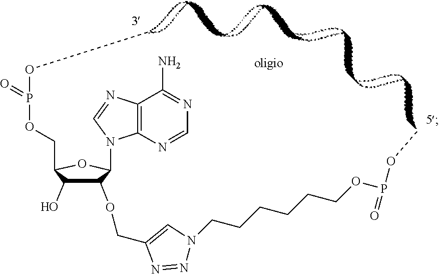
Apy refers to 2′-O-propynyl-adenosine-3′-phosphate: 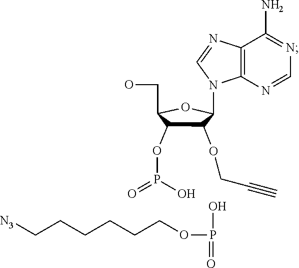
Q301 refers to 6-azidohexyl phosphate:

Oligonucleotides were synthesized on a MerMade-12 DNA/RNA synthesizer. Sterling solvents/reagents from Glen Research, 500-Å controlled pore glass (CPG) solid supports (Prime Synthesis), 2′-deoxy 3′-phosphoramidites (Thermo), and 2′-O-methyl (2′-OMe), 2′-deoxy-2′-fluoro (2′-F) ribonucleoside 3′-phosphoramidites (Hongene) were all used as received. The 2′-OMe-uridine-51-bis-POM-(E) vinylphosphonate (VP) 3′-phosphoramidite (synthesized according to the procedures described in Parmar et al., “Facile synthesis, geometry, and 2′-substituent-dependent in vivo activity of 5′-(E)- and 5′-(Z)-vinylphosphonate-modified siRNA conjugates,” J. Med. Chem., 61: 734-44 (2018), which is incorporated herein by reference in its entirety) was dissolved to 0.15 M in 85% acetonitrile 15% dimethylformamide (DMF) and coupled using standard conditions on the synthesizer. GalNAc CPG support (L, Scheme I) was prepared and used as described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety. 5-Bromohexyl phosphoramidite (Glen Research, Cat #10-1946) was dissolved to 0.15 M in acetonitrile and coupled using standard conditions on the synthesizer. 3′-alkyne CPG support and 3′-alkyne hydroxyprolinol phosphoramidite (Y, Scheme I) was prepared and used as described in Jayaprakash et al., “Non-nucleoside building blocks for copper-assisted and copper-free click chemistry for the efficient synthesis of RNA conjugates,” Org. Lett., 12: 5410-13 (2010), which is incorporated herein by reference in its entirety. Low-water content acetonitrile was purchased from EMD Chemicals. DNA and RNA oligonucleotides were synthesized using modified synthesis cycles, based on those provided with the instrument. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M 12 in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase syntheses (SPS), the CPG solid support was washed with 5% (v/v) piperidine in anhydrous acetonitrile three times with 5-minute holds after each flow. The support was then washed with anhydrous acetonitrile and dried with argon. The oligonucleotides were then incubated with 28-30% (w/v) NH₄OH at 35° C. for 20 hours. For VP-containing oligonucleotides, after completion of the SPS, the CPG solid support was incubated with 28-30% (w/v) NH₄OH, where 5% of (v/v) of diethylamine was added at 35° C. for 20 hours (see O'Shea et al., “An efficient deprotection method for 5′-[O,O-bis(pivaloyloxymethyl)]-(E)-vinylphosphonate containing oligonucleotides,” Tetrahedron, 74: 6182-86 (2018), which is incorporated herein by reference in its entirety). The solvent was collected by filtration and the support was rinsed with water prior to analysis. Oligonucleotide solutions of ˜1 OD₂₆₀ units/mL were used for analysis of the crudes, where 30-50 μL of solution were injected. LC/ESI-MS was performed on an Agilent 6130 single quadrupole LC/MS system using an XBridge C₈ column (2.1×50 mm, 2.5 μm) at 60° C. Buffer A consisted of 200 mM 1,1,1,3,3,3-hexafluoro-2-propanol (HFIP) and 16.3 mM triethylamine (TEA) in water, and buffer B was 100% methanol. A gradient from 0% to 40% of buffer B over 10 minutes followed by washing and recalibration at a flow rate of 0.70 mL/min. The column temperature was 75° C.

All oligonucleotides were purified and desalted, and further annealed to form GalNAc-siRNAs, based on the procedures described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety.

Circulation of Sense-Strand Oligonucleotides and Formation of sciRNA-GalNAc Conjugates 5′-azido and “Click” Chemistry Synthesis for Circular Oligonucleotides.

To functionalize the sense strand, commercially available 5-bromohexyl phosphoramidite was coupled to the 5′ end during solid-phase synthesis. To provide the 6-azidohexyl click chemistry “handle”, 10 μmol of CPG loaded with 5′-(5-bromohexyl) modified oligonucleotides (see Lietard et al., “An efficient reagent for 5′-azido oligonucleotide synthesis,” Tetrahedron Lett., 48, 8795-98 (2007), which is incorporated herein by reference in its entirety) were suspended in 15 mL of an anhydrous DMF solution containing 130 mg of sodium azide and 300 mg of sodium iodide. The mixture was vigorously shaken at 65° C. for 75 minutes. After cooling down, the solution was filtered off and the CPG beads with the resulting 5′-(5-azidohexyl) solid-supported oligonucleotides were washed with DMF (2×10 mL) and dried under a stream of argon. The oligonucleotides were released from the solid support and purified and desalted as described above. The oligonucleotides were then dissolved in water to a concentration of ˜10 OD₂₆₀ units/mL. For a copper(I)-catalyzed alkyne-azide cycloaddition (CuAAC) “click” cyclization reaction yielding Z (Scheme I), 2 mL of the oligonucleotide solution (˜200 OD₂₆₀ units) were mixed with 2 mL of methanol, 1.1 mL of sodium L-ascorbate (0.1 mM) and 1.1 mL of copper sulfate (20 mM). The reaction mixture was placed into a microwave (MW) tube container, equipped with a stirring bar, and placed in a MW reactor for 40 minutes at 60° C. (power ˜8 W, stirring, cooling, P═0). This protocol is a modification of that described in Lietard et al., “New strategies for cyclization and bicyclization of oligonucleotides by click chemistry assisted by microwaves,” J. Org. Chem., 73, 191-200 (2008), which is incorporated herein by reference in its entirety. All cyclic oligonucleotides were purified and desalted, and further annealed with an antisense strand to form the GalNAc-sciRNA as described above.

1. A small circular interfering RNA (sciRNA) comprising a sense strand and an antisense strand, wherein each of the sense and antisense strands comprises at least one nucleic acid modification, and wherein the sciRNA comprises one or more of the following:
(a) the antisense strand comprises a phosphate mimic at the 5′-end of an antisense nucleotide sequence, selected from the group consisting of 5′-phosphorothioate (5′-PS), 5′-phosphorodithioate (5′-PS₂), 5′-vinylphosphonate (5′-VP), 5′-methylphosphonate (5′-MePhos), and 5′-deoxy-5′-C-malonyl;
(b) all the nucleotides in the sense strand are modified; and
(c) all the nucleotides in the antisense strand are modified.

2. The sciRNA of claim 1, wherein the sense strand or antisense strand has a circular or substantially circular structure.

3. (canceled)

4. The sciRNA of claim 1, wherein the sense strand is at least 20 nucleotides in length.

5. The sciRNA of claim 4, wherein the sense strand comprises at least one sense nucleotide sequence, having about 20 to about 45 nucleotides in length.

6. The sciRNA of claim 5, wherein the sense strand is annealed with an antisense strand having about 19 to about 23 nucleotides in length, complementary to a target mRNA transcript nucleotide sequence.

7-10. (canceled)

11. The sciRNA of claim 1, further comprising one or more ligands.

12. The sciRNA of claim 11, wherein at least one of the ligands is a lipophilic moiety.

13-14. (canceled)

15. The sciRNA of claim 11, wherein at least one of the ligands is a carbohydrate-based ligand.

16. (canceled)

17. The sciRNA of claim 15, wherein the carbohydrate-based ligand is one or more GalNAc derivatives attached through a bivalent or trivalent branched linker.

18. (canceled)

19. The sciRNA of claim 11, wherein at least one of the ligands is conjugated with a sense nucleotide sequence of the sense strand, at the 3′-end, 5′-end, or an internal position of the sense nucleotide sequence.

20. The sciRNA of claim 11, wherein at least one of the ligands is conjugated with an antisense nucleotide sequence of the antisense strand, at the 3′-end, 5′-end, or an internal position of the antisense nucleotide sequence.

21. The sciRNA of claim 1, wherein the sciRNA further comprises at least one chemical modification selected from the group consisting of an internucleoside linkage modification, a nucleobase modification, a sugar modification, and combinations thereof.

22. The sciRNA of claim 21, wherein the chemical modification is selected from the group consisting of LNA, ENA, HNA, CeNA, 2′-methoxyalkyl, 2′-O-alkyl, 2′-O-allyl, 2′-C-allyl, 2′-fluoro, 2′-deoxy, 2′-O-N-methylacetamido (2′-O-NMA), 2′-O-dimethylaminoethoxyethyl (2′-O-DMAEOE), 2′-O-aminopropyl (2′-O-AP), 2′-ara-F, L-nucleoside modification, and combinations thereof.

23. The sciRNA of claim 1, wherein each of the nucleotide in the sense strand or antisense strand is independently modified with a 2′-modification selected from the group consisting of 2′-O-methyl, 2′-deoxy, 2′-fluoro, and combinations thereof.

24-36. (canceled)

37. The sciRNA of claim 2, wherein the sense or antisense strand forms circular or substantially circular structure via a cycling linking moiety that connects one end of the sense or antisense strand to the other end of the sense or antisense strand.

38. The sciRNA of claim 37, wherein the cycling linking moiety contains one or more linkages selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, aethylene oxide linkage, and combinations thereof.

39. The sciRNA of claim 37, wherein the cycling linking moiety contains one or more cyclic groups selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl.

40. (canceled)

41. The sciRNA of claim 1, wherein the phosphate mimic is a 5′-vinyl phosphonate (VP).

42. The sciRNA of claim 1, wherein the sciRNA comprises at least two blocks of two consecutive phosphorothioate or methylphosphonate internucleotide linkage modifications.

43. The sciRNA of claim 42, wherein the antisense strand comprises at least two consecutive phosphorothioate internucleotide linkage modifications within positions 18-23 of an antisense nucleotide sequence, counting from the 5′-end of the antisense nucleotide sequence; and the sense strand comprises at least two consecutive phosphorothioate internucleotide linkage modifications within position 1-5 of the sense nucleotide sequence, counting from the 5′-end of the sense nucleotide sequence.

44. The sciRNA of claim 1, wherein:
the sciRNA comprises at least two blocks of two consecutive phosphorothioate or methylphosphonate internucleotide linkage modifications, and
the sciRNA comprises one or more ligands.

45. The sciRNA of claim 44, wherein:
all the nucleotides in the sense strand and antisense strand are modified with a 2′-O-methyl or 2′-fluoro modification;
the antisense strand comprises at least two consecutive phosphorothioate internucleotide linkage modifications within positions 18-23 of an antisense nucleotide sequence, counting from the 5′-end of the antisense nucleotide sequence; and the sense strand comprises at least two consecutive phosphorothioate internucleotide linkage modifications within position 1-5 of the sense nucleotide sequence, counting from the 5′-end of the sense nucleotide sequence; and
the sciRNA comprises at least one carbohydrate-based ligand conjugated with a sense nucleotide sequence of the sense strand, at the 3′-end of the sense nucleotide sequence.

46. The sciRNA of claim 2, wherein a duplex region is formed between the sense strand and antisense strand at least at the seed region of the antisense strand.

47. A pharmaceutical composition comprising the sciRNA of claim 1 and a pharmaceutically acceptable excipient.

48. A method for inhibiting the expression of a target gene in a subject, comprising:
administering to the subject the sciRNA of claim 1, in an amount sufficient to inhibit expression of the target gene.
